(19) United States
(12) Patent Application Publication
(10) Pub. No.: US 2022/0268790 A1
(43) Pub. Date: Aug. 25, 2022

(54) EVALUATING METHOD FOR PHARMACOLOGICAL ACTIONS OF IMMUNE CHECKPOINT INHIBITORS, CALCULATING METHOD, EVALUATING APPARATUS, CALCULATING APPARATUS, EVALUATING PROGRAM, CALCULATING PROGRAM, RECORDING MEDIUM, EVALUATING SYSTEM, AND TERMINAL APPARATUS

(71) Applicant: AJINOMOTO CO., INC., Tokyo (JP)

(72) Inventors: Sachise KARAKAWA, Kanagawa (JP); Tomoyuki TAGAMI, Kanagawa (JP); Rumi NISHIMOTO, Kanagawa (JP); Shinya KIKUCHI, Kanagawa (JP); Akira IMAIZUMI, Kanagawa (JP); Koichi AZUMA, Fukuoka (JP); Tomoaki HOSHINO, Fukuoka (JP); Takaaki TOKITO, Fukuoka (JP); Hidenobu ISHII, Fukuoka (JP); Norikazu MATSUO, Fukuoka (JP)

(73) Assignee: AJINOMOTO CO., INC., Tokyo (JP)

(21) Appl. No.: 17/730,484

(22) Filed: Apr. 27, 2022

Related U.S. Application Data
(63) Continuation of application No. PCT/JP2020/041614, filed on Nov. 6, 2020.

(30) Foreign Application Priority Data
Nov. 8, 2019 (JP) 2019-203363

(51) Int. Cl. G01N 33/94; G01N 33/68; G16H 10/40; G16H 50/30

(52) U.S. Cl. CPC G01N 33/94; G01N 33/68; G01N 2800/52; G16H 50/30; G16H 10/40

(57) ABSTRACT

An evaluating method includes an evaluating step of evaluating a pharmacological action of an immune checkpoint inhibitor in a subject to be evaluated, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hAnthA, hIAA, hKyn, hTrp, IAA, ILA, Kyn, KynA, NFKyn, NP, PA, QA, Serot, and XA in blood of the subject to be evaluated, or (ii) a value of a formula calculated using the concentration value and the formula including an explanatory variable to be substituted with the concentration value.

### CROSS-REFERENCE TO RELATED APPLICATION

This application is based upon and claims the benefit of priority from PCT Application PCT/JP2020/041614, filed Nov. 6, 2020, which claims priority from Japanese Patent Application No. 2019-203363, filed Nov. 8, 2019, the entire contents of which are incorporated herein by reference.

### BACKGROUND OF THE INVENTION

### 1. Field of the Invention

The present invention relates to an evaluating method for pharmacological actions of immune checkpoint inhibitors, a calculating method, an evaluating apparatus, a calculating apparatus, an evaluating program, a calculating program, a recording medium, an evaluating system, and a terminal apparatus.

### 2. Description of the Related Art

Immune checkpoint inhibitor therapy is a method of treating cancer by clearing the immunosuppressive state caused by cancer and normalizing the immune function of the body. The efficacy of this therapy has been shown in various types of cancer, including malignant melanoma, non-small cell lung cancer, small cell lung cancer, malignant pleural mesothelioma, renal cell carcinoma, Hodgkin lymphoma, Merkel cell carcinoma, urothelial carcinoma, head and neck cancer, esophageal cancer, gastric cancer, hepatocellular carcinoma, breast cancer, and bladder cancer. There have been many reports on the outcome of immune checkpoint inhibitor therapy, for example, including cure cases even in advanced and recurrent cancers difficult to be cured with conventional therapies. Immune checkpoint inhibitor therapy is becoming popular as a fourth treatment method in addition to conventional therapies such as surgical therapy, radiotherapy, and chemotherapy.

Unfortunately, immune checkpoint inhibitor therapy has found to have several disadvantages. Specifically, while some patients respond well to the treatment, only about 20 to 30% of patients show long-term effects, immune-related adverse events (irAEs) appear, and medical costs are higher than with conventional therapies. For these reasons, there has been a need for diagnostic techniques to select patients who should be treated with immune checkpoint inhibitor therapy.

In conventional companion diagnostics for use to choose immune checkpoint inhibitor therapy, indicators obtained from tumor tissues (for example, the presence/absence of immunosuppressive molecules such as PD-L1 molecules or oncogene mutations, or microsatellite instability) are mainly used for patient treatment choice. However, the mechanism of suppressing the cancer immune response has been reported to involve interactions with various immunosuppressive molecules and immunosuppressive cell function. For accurate prediction of therapeutic effects, there has been a demand for developing new biomarkers.

Amino acids are common nutrients used as substrates and energy sources for biological components such as proteins and nucleic acids and are essential for regulation of cancer cell proliferation and immune cell function. Increased amino acid utilization in energy metabolism due to cancer cell proliferation, protein catabolism in systemic organs, and amino acid metabolism disorders mediated by various immunomodulatory cells are thought to cause a competitive state of amino acids required for immune responses to cancer, such as cysteine, glutamine, phenylalanine, tryptophan, and arginine and lead to immune evasion in cancer (“Sikalidis A K., Amino Acids and Immune Response: A Role for Cysteine, Glutamine, Phenylalanine, Tryptophan and Arginine in T-cell Function and Cancer?, Pathol Oncol Res., 2015: 21: 9”). The mechanism of interactions between cancer and immunity have been reported. For example, arginine-degrading enzymes (arginase) and tryptophan-metabolizing enzymes (IDO) expressed on myeloid-derived suppressor cells (MDSC) and tumor-infiltrating macrophages cause depletion of amino acids and consequently suppress immune cell function, and amino acid-metabolizing enzymes such as IDO and histidine decarboxylase (HDC) are involved in function regulation of regulatory T cells (Treg) (“Sikalidis A K., Amino Acids and Immune Response: A Role for Cysteine, Glutamine, Phenylalanine, Tryptophan and Arginine in T-cell Function and Cancer?, Pathol Oncol Res., 2015: 21: 9”, “Raber P, Ochoa A C, Rodriguez P C., Metabolism of L-arginine by myeloid-derived suppressor cells in cancer: mechanisms of T cell suppression and therapeutic perspectives., Immunol Invest., 2012; 41(6-7): 614”, “Sharma M D, Baban B, Chandler P et al., Plasmacytoid dendritic cells from mouse tumor-draining lymph nodes directly activate mature Tregs via indoleamine 2,3-dioxygenase., J Clin Invest., 2007; 117(9): 2570”, and “Tamaka K, Seike M, Hagiwara T et al., Histamine suppresses regulatory T cells mediated by TGF-β in murine chronic allergic contact dermatitis., Exp Dermatol., 2015; 24(4): 280”). A method of evaluating the function of MDSC on the life prognosis of critically ill patients by analyzing arginine in the blood has been proposed (“Gey A, Tadie J M, Caumont-Prim A et al., Granulocytic myeloid-derived suppressor cells inversely correlate with plasma arginine and overall survival in critically ill patients., Clinical and Experimental Immunology, 2014; 180: 280-288”). More recently, quinolinic acid, which is a tryptophan metabolite mediated by IDO, and the combination of tryptophan and kynurenine have been reported as indicators for predicting the prognosis of immune checkpoint inhibitor therapy (“Botticelli A, Cerbelli B, Lionetto L et al., Can IDO activity predict primary resistance to anti-PD-1 treatment in NSCLC?, J Transl Med., 2018; 16(1): 219”). There have been reports that activation of the tryptophan metabolic pathway can be an indicator of the prognosis of immune checkpoint inhibitor therapy even in malignant melanoma and renal cell carcinoma, suggesting that a common immunosuppressive mechanism may work in various types of cancer (“Li H, Bullock K, Gurjao C et al., Metabolomic adaptations and correlates of survival to immune checkpoint blockade., Nat Commun., 2019; 10(1): 4346”).

WO 2013/168550 directed to a method of evaluating therapeutic effects of cancer immunotherapy using amino acid concentrations has been published.

The highly accurate discrimination or prediction of the prognosis of treatment and the risk of developing an adverse effect before the start or at an early stage after the start of immune checkpoint inhibitor therapy enables each individual patient to choose a suitable therapy more correctly and leads to improvement of the result of immune checkpoint inhibitor therapy. Patients for whom ineffectiveness of immune checkpoint inhibitor therapy is predicted has the benefit of avoiding unnecessary medical costs and adverse effects as well as the benefit of having the opportunity to choose another treatment at an early stage. The promotion of personalized medicine is also expected to greatly help improve the cost efficiency of healthcare.

Unfortunately, “Sikalidis A K., Amino Acids and Immune Response: A Role for Cysteine, Glutamine, Phenylalanine, Tryptophan and Arginine in T-cell Function and Cancer?, Pathol Oncol Res., 2015: 21: 9”, “Raber P, Ochoa A C, Rodriguez P C., Metabolism of L-arginine by myeloid-derived suppressor cells in cancer: mechanisms of T cell suppression and therapeutic perspectives., Immunol Invest., 2012; 41(6-7): 614”, “Sharma M D, Baban B, Chandler P et al., Plasmacytoid dendritic cells from mouse tumor-draining lymph nodes directly activate mature Tregs via indoleamine 2,3-dioxygenase., J Clin Invest., 2007; 117(9): 2570”, and “Tamaka K, Seike M, Hagiwara T et al., Histamine suppresses regulatory T cells mediated by TGF-β in murine chronic allergic contact dermatitis., Exp Dermatol., 2015; 24(4): 280” do not discuss the possibility of evaluating local changes in immune cell function using metabolites in the blood. “Gey A, Tadie J M, Caumont-Prim A et al., Granulocytic myeloid-derived suppressor cells inversely correlate with plasma arginine and overall survival in critically ill patients., Clinical and Experimental Immunology, 2014; 180: 280-288” does not relate to the evaluation of local immune response by cancer. “Botticelli A, Cerbelli B, Lionetto L et al., Can IDO activity predict primary resistance to anti-PD-1 treatment in NSCLC?, J Transl Med., 2018; 16(1): 219” does not provide sufficient discrimination accuracy. WO 2013/168550 does not indicate the possibility of predicting the prognosis of immune checkpoint inhibitor therapy.

In short, biomarkers using amino acids or amino acid-related metabolites in blood that can highly accurately discriminate or predict the prognosis of treatment or the risk of developing an adverse effect of immune checkpoint inhibitors have not yet developed.

### SUMMARY OF THE INVENTION

It is an object of the present invention to at least partially solve the problems in the conventional technology.

The present invention was made in view of the problem.

It is an object of the present invention to provide an evaluation method, a calculating method, an evaluating apparatus, a calculating apparatus, an evaluating program, a calculating program, a recording medium, an evaluating system, and a terminal apparatus that can provide highly reliable information helpful in identifying individual differences in pharmacological actions of immune checkpoint inhibitors.

In order to solve the problem and achieve the object, an evaluating method according to the present invention includes an evaluating step of evaluating a pharmacological action of an immune checkpoint inhibitor in a subject to be evaluated, using (i) a concentration value of at least one metabolite among 21 kinds of amino acids (Glu, Arg, Orn, Cit, His, Val, Phe, Tyr, Met, Pro, Asn, Leu, Lys, Thr, Ile, Gln, Ala, Ser, a-ABA, Trp, and Gly) and 15 kinds of amino acid-related metabolites (AnthA, hAnthA, hIAA, hKyn, hTrp, IAA, ILA, Kyn, KynA, NFKyn, NP, PA, QA, Serot, and XA) in blood of the subject to be evaluated, or (ii) a value of a formula calculated using the concentration value and the formula including an explanatory variable to be substituted with the concentration value.

In the present description, immune checkpoint inhibitors include PD-1 inhibitors (such as nivolumab or pembrolizumab), PD-L1 inhibitors (such as atezolizumab or durvalumab), and CTLA-4 inhibitors (such as ipilimumab). In the present description, pharmacological actions include drug pharmacological actions (main effects) and general pharmacological actions (adverse effects).

In the present description, various amino acids and various amino acid-related metabolites are mainly written in abbreviations, the formal names of these are as follows.

| (Abbreviation) | (Formal name) |
|---|---|
| Ala | Alanine |
| AnthA | Anthranic Acid |
| Arg | Arginine |
| Asn | Asparagine |
| ABA | α-Aminobutyric acid |
| Cit | Citrulline |
| Gln | Glutamine |
| Glu | Glutamic acid |
| Gly | Glycine |
| hAnthA | 3-Hydroxy Anthranilic Acid |
| hIAA | 5-Hydroxy Indol 3-Acetic Acid |
| His | Histidine |
| hKyn | hydroxy Kynurenine |
| hTrp | 5-hydroxy Tryptophan |
| IAA | Indol 3-Acetic Acid |
| ILA | Indol 3-Lactic Acid |
| Ile | Isoleucine |
| Kyn | Kynurenine |
| KynA | Kynurenic Acid |
| Leu | Leucine |
| Lys | Lysine |
| Met | Methionine |
| NFKyn | N-Formyl Kynurenine |
| NP | Neopterin |
| Orn | Ornithine |
| PA | Picolinic Acid |
| Phe | Phenylalanine |
| Pro | Proline |
| QA | Quinolinic Acid |
| Ser | Serine |
| Serot | Serotonine |
| Thr | Threonine |
| Trp | Tryptophan |
| Tyr | Tyrosine |
| Val | Valine |
| XA | Xanthurenic Acid |

In the evaluating method according to the present invention, the blood is taken from the subject to be evaluated after or before treatment with the immune checkpoint inhibitor is started, and the evaluating step is to evaluate the effect of the treatment in the subject to be evaluated.

In the present description, “before treatment is started” may be referred to as “before treatment” or “before the start of treatment”, and “after treatment is started” may be referred to as “after the start of treatment”. In the present description, “before the start of treatment” includes, for example, before initial treatment in a narrow sense in treatment over a certain period of time in a broad sense is performed. In the present description, “after the start of treatment” includes, for example, after initial treatment in a narrow sense in treatment over a certain period of time in a broad sense is performed and before final treatment in a narrow sense is performed (for example, generally called “during treatment”), or after final treatment in a narrow sense in treatment over a certain period of time in a broad sense is performed (for example, generally called “after treatment”).

In the evaluating method according to the present invention, the blood is taken from the subject to be evaluated before treatment with the immune checkpoint inhibitor is started, and the evaluating step is to evaluate a risk of developing an adverse effect with the treatment in the subject to be evaluated.

In the evaluating method according to the present invention, the evaluating step is performed by a control unit of an information processing apparatus including the control unit.

A calculating method according to the present invention includes a calculating step of calculating a value of a formula for evaluating a pharmacological action of an immune checkpoint inhibitor, using (i) a concentration value of at least one metabolite among the 21 kinds of amino acids and the 15 kinds of amino acid-related metabolites in blood of a subject to be evaluated, and (ii) the formula including an explanatory variable to be substituted with the concentration value.

In the calculating method according to the present invention, the blood is taken from the subject to be evaluated after or before treatment with the immune checkpoint inhibitor is started, and the formula is to evaluate an effect of the treatment.

In the calculating method according to the present invention, the blood is taken from the subject to be evaluated before treatment with the immune checkpoint inhibitor is started, and the formula is to evaluate a risk of developing an adverse effect with the treatment.

In the calculating method according to the present invention, the calculating step is performed by a control unit of an information processing apparatus including the control unit.

An evaluating apparatus according to the present invention is an evaluating apparatus including a control unit. The control unit includes an evaluating unit that evaluates a pharmacological action of an immune checkpoint inhibitor in a subject to be evaluated, using (i) a concentration value of at least one metabolite among the 21 kinds of amino acids and the 15 kinds of amino acid-related metabolites in blood of the subject to be evaluated, or (ii) a value of a formula calculated using the concentration value and the formula including an explanatory variable to be substituted with the concentration value.

In the evaluating apparatus according to the present invention, the evaluating apparatus is communicatively connected to a terminal apparatus via a network. The terminal apparatus provides concentration data on the concentration value or the value of the formula. The control unit further includes a data receiving unit that receives the concentration data or the value of the formula transmitted from the terminal apparatus, and a result transmitting unit that transmits an evaluation result obtained by the evaluating unit to the terminal apparatus. The evaluating unit uses the concentration value included in the concentration data or the value of the formula received by the data receiving unit.

A calculating apparatus according to the present invention is a calculating apparatus including a control unit. The control unit includes a calculating unit that calculates a value of a formula for evaluating a pharmacological action of an immune checkpoint inhibitor, using (i) a concentration value of at least one metabolite among the 21 kinds of amino acids and the 15 kinds of amino acid-related metabolites in blood of a subject to be evaluated, and (ii) the formula including an explanatory variable to be substituted with the concentration value.

An evaluating program according to the present invention is an evaluating program for causing an information processing apparatus including a control unit to perform an evaluating method. The evaluating method includes an evaluating step of evaluating a pharmacological action of an immune checkpoint inhibitor in a subject to be evaluated, using (i) a concentration value of at least one metabolite among the 21 kinds of amino acids and the 15 kinds of amino acid-related metabolites in blood of the subject to be evaluated, or (ii) a value of a formula calculated using the concentration value and the formula including an explanatory variable to be substituted with the concentration value.

A calculating program according to the present invention is a calculating program for causing an information processing apparatus including a control unit to perform a calculating method. The calculating method includes a calculating step of calculating a value of a formula for evaluating a pharmacological action of an immune checkpoint inhibitor, using (i) a concentration value of at least one metabolite among the 21 kinds of amino acids and the 15 kinds of amino acid-related metabolites in blood of a subject to be evaluated, and (ii) the formula including an explanatory variable to be substituted with the concentration value.

A recording medium according to the present invention is a computer readable recording medium encoded with the evaluating program or the calculating program. Specifically, the recording medium according to the present invention is a non-transitory tangible computer readable recording medium including programmed instructions for causing, when executed by an information processing apparatus, the information processing apparatus to perform the evaluating method or the calculating method.

An evaluating system according to the present invention is an evaluating system including an evaluating apparatus including a control unit and a terminal apparatus including a control unit that are communicatively connected to each other via a network. The control unit of the terminal apparatus includes (I) a data transmitting unit that transmits, to the evaluating apparatus, (i) concentration data on a concentration value of at least one metabolite among the 21 kinds of amino acids and the 15 kinds of amino acid-related metabolites in blood of a subject to be evaluated, or (ii) a value of a formula calculated using the concentration value and the formula including an explanatory variable to be substituted with the concentration value, and (II) a result receiving unit that receives an evaluation result transmitted from the evaluating apparatus on a pharmacological action of an immune checkpoint inhibitor. The control unit of the evaluating apparatus includes (I) a data receiving unit that receives the concentration data or the value of the formula transmitted from the terminal apparatus, (II) an evaluating unit that evaluates a pharmacological action of an immune checkpoint inhibitor in the subject to be evaluated, using the concentration value included in the concentration data or the value of the formula received by the data receiving unit, and (III) a result transmitting unit that transmits the evaluation result obtained by the evaluating unit to the terminal apparatus.

A terminal apparatus according to the present invention is a terminal apparatus including a control unit. The control unit includes a result obtaining unit that obtains an evaluation result on a pharmacological action of an immune checkpoint inhibitor. The evaluation result is a result of evaluating a pharmacological action of an immune checkpoint inhibitor in a subject to be evaluated, using (i) a concentration value of at least one metabolite among the 21 kinds of amino acids and the 15 kinds of amino acid-related metabolites in blood of the subject to be evaluated, or (ii) a value of a formula calculated using the concentration value and the formula including an explanatory variable to be substituted with the concentration value.

The terminal apparatus according to the present invention is communicatively connected via a network to an evaluating apparatus that evaluates a pharmacological action of an immune checkpoint inhibitor. The control unit includes a data transmitting unit that transmits concentration data on the concentration value or the value of the formula to the evaluating apparatus. The result obtaining unit receives the evaluation result transmitted from the evaluating apparatus.

The present invention achieves the effect of providing highly reliable information helpful in identifying individual differences in pharmacological actions of immune checkpoint inhibitors.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a principle configurational diagram showing a basic principle of a first embodiment;

FIG. 2 is a principle configurational diagram showing a basic principle of a second embodiment;

FIG. 3 is a diagram showing an example of an entire configuration of a present system;

FIG. 4 is a diagram showing another example of an entire configuration of the present system;

FIG. 5 is a block diagram showing an example of a configuration of an evaluating apparatus 100 in the present system;

FIG. 6 is a chart showing an example of information stored in a concentration data file 106a;

FIG. 7 is a chart showing an example of information stored in an index state information file 106b;

FIG. 8 is a chart showing an example of information stored in a designated index state information file 106c;

FIG. 9 is a chart showing an example of information stored in a formula file 106d1;

FIG. 10 is a chart showing an example of information stored in an evaluation result file 106e;

FIG. 11 is a block diagram showing a configuration of an evaluating part 102d;

FIG. 12 is a block diagram showing an example of a configuration of a client apparatus 200 in the present system;

FIG. 13 is a block diagram showing an example of a configuration of a database apparatus 400 in the present system;

FIG. 14 is a table depicting the concentration values of amino acids and amino acid-related metabolites, and the like;

FIG. 15 is a table depicting analysis results obtained in multivariate analysis using a univariate Cox hazards model;

FIG. 16 is a table depicting analysis results obtained in multivariate analysis using a univariate Cox hazards model;

FIG. 17 is a diagram depicting distributions of C-Index for discriminants with two explanatory variables, discriminants with three explanatory variables, discriminants with four explanatory variables, discriminants with five explanatory variables, and discriminants with six explanatory variables;

FIG. 18 is a table depicting the frequency of occurrence of amino acid explanatory variables in discriminants;

FIG. 19 is a table depicting the frequency of occurrence of amino acid explanatory variables and amino acid-related metabolite explanatory variables in discriminants;

FIG. 20 is a table depicting the frequency of occurrence of amino acid explanatory variables in discriminants;

FIG. 21 is a table depicting the frequency of occurrence of amino acid explanatory variables and amino acid-related metabolite explanatory variables in discriminants;

FIG. 22 is a graph for explaining the discriminant performance of a discriminant prepared to minimize Akaike's information criterion (AIC) by the stepwise method;

FIG. 23 is a table depicting analysis results obtained in multivariate analysis using a univariate logistic regression model;

FIG. 24 is a diagram depicting distributions of AOC_AUC for discriminants with two explanatory variables, discriminants with three explanatory variables, discriminants with four explanatory variables, discriminants with five explanatory variables, and discriminants with six explanatory variables;

FIG. 25 is a table depicting the frequency of occurrence of amino acid explanatory variables in discriminants;

FIG. 26 is a table depicting the frequency of occurrence of amino acid explanatory variables and amino acid-related metabolite explanatory variables in discriminants;

FIG. 27 is a table depicting analysis results obtained in multivariate analysis using a univariate Cox hazards model; and

FIG. 28 is a table depicting analysis results obtained in univariate correlation analysis.

### DETAILED DESCRIPTION OF THE PREFERRED EMBODIMENTS

An embodiment (first embodiment) of an evaluating method according to the present invention and an embodiment (second embodiment) of an evaluating apparatus, an evaluating method, an evaluating program, a recording medium, an evaluating system, and a terminal apparatus according to the present invention will be explained in detail below with reference to the drawings. The present invention is not limited by these embodiments.

### First Embodiment

### 1-1. Overview of First Embodiment

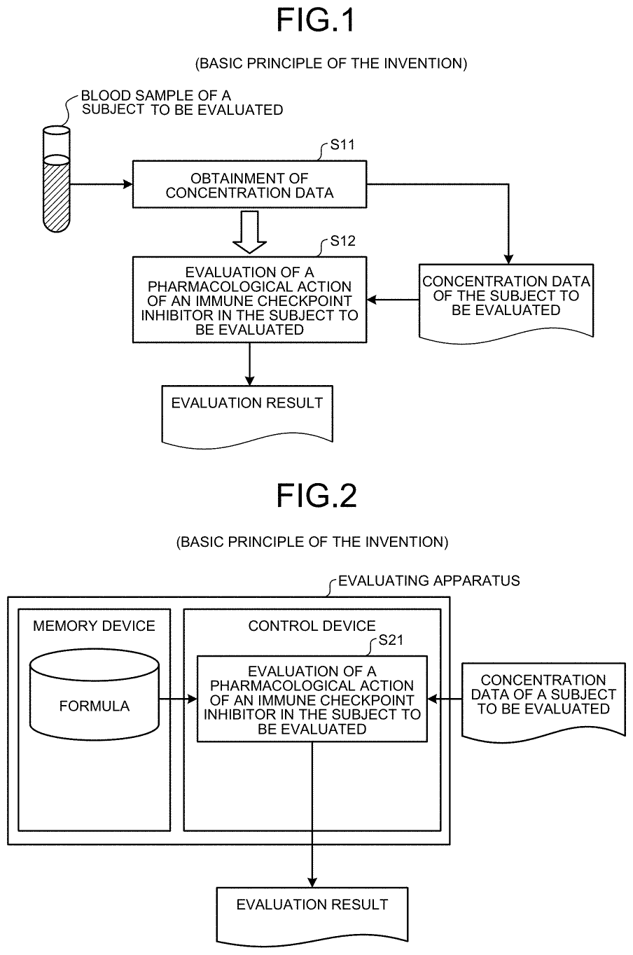

An overview of the first embodiment will be explained with reference to FIG. 1. FIG. 1 is a principle configuration diagram showing a basic principle according to the first embodiment.

Concentration data on a concentration value of at least one metabolite among the 21 kinds of amino acids and the 15 kinds of amino acid-related metabolites in blood (for example, including plasma and serum) taken from a subject with cancer to be evaluated (for example, an individual such as an animal or a human) that may be treated with an immune checkpoint inhibitor is obtained (step S11).

As used herein “may be treated” means, for example, that treatment may be chosen or treatment is scheduled.

At step S11, for example, one or both of concentration data derived from blood taken from the subject to be evaluated before cancer treatment (for example, treatment by surgical therapy, chemotherapy, radiotherapy, or cancer immunotherapy) is started (concentration data before the start of treatment) and concentration data derived from blood taken after the treatment is started (concentration data after the start of treatment) may be obtained. The term “before the start of treatment” includes, for example, before initial treatment in a narrow sense in treatment over a certain period of time in a broad sense is performed. The term “after the start of treatment” includes, for example, after initial treatment in a narrow sense in treatment over a certain period of time in a broad sense is performed and before final treatment in a narrow sense is performed (for example, generally called “during treatment”), or after final treatment in a narrow sense in treatment over a certain period of time in a broad sense is performed (for example, generally called “after treatment”).

At Step S11, for example, the concentration data measured by a company or the like that measures concentrations may be obtained. For example, the following measuring method of (A), (B), or (C) may be used to measure concentrations from the blood extracted from the subject to be evaluated to obtain the concentration data. In the present description, the unit of the concentration may be, for example, molar concentration, weight concentration, enzyme activity, or one obtained by addition, subtraction, multiplication, and division of any constant with these concentrations.

(A) Plasma is separated from blood by centrifuging the collected blood sample. All plasma samples are frozen and stored at −80° C. until the concentration is measured. At the time of measuring the concentration, after acetonitrile is added to deproteinize the plasma samples, impurities such as phospholipid are removed by solid phase extraction as needed, pre-column derivatization is then performed using a labeling reagent (3-aminopyridyl-N-hydroxysuccinimidyl carbamate), and the concentration is analyzed by liquid chromatograph mass spectrometry (LC/MS) (see International Publication WO 2003/069328, International Publication WO 2005/116629, or Nonpatent Literature “Chromatography 2019, 40, 127-133”).
(B) Plasma is separated from blood by centrifuging the collected blood sample. All plasma samples are frozen and stored at −80° C. until the concentration is measured. At the time of measuring the concentration, sulfosalicylic acid is added to deproteinize the plasma samples, and the concentration is analyzed by an amino acid analyzer based on post-column derivatization using a ninhydrin reagent.
(C) Blood cell separation is performed on the collected blood sample by using a membrane, micro-electro-mechanical System (MEMS) technology, or the principle of centrifugation, whereby plasma or serum is separated from the blood. A plasma or serum sample the concentration of which is not measured immediately after obtaining the plasma or the serum is frozen and stored at −80° C. until the concentration is measured. At the time of measuring the concentration, a molecule that reacts with or binds to a target substance in blood, such as an enzyme or an aptamer, is used to perform quantitative analysis and the like on an increasing or decreasing substance or a spectroscopic value by substrate recognition, whereby the concentration is analyzed.

Subsequently, a pharmacological action of the immune checkpoint inhibitor in the subject to be evaluated is evaluated (predicted) using the concentration value included in the concentration data obtained at step S11 (step S12). Before step S12 is performed, data such as defectives and outliers may be removed from the concentration data obtained at step S11. As used herein “a pharmacological action of the immune checkpoint inhibitor in the subject to be evaluated is evaluated” means, for example, that a pharmacological action of the immune checkpoint inhibitor developed in the subject to be evaluated is evaluated. When both of the concentration data before the start of treatment and the concentration data after the start of treatment are used at step S12, for example, the ratio or the difference between the concentration value before the start of treatment and the concentration value after the start of treatment may be calculated, and the calculated ratio or difference may be used to perform evaluation. At step S12, the effect of treatment (prognosis of treatment) with the immune checkpoint inhibitor in the subject to be evaluated may be evaluated using the concentration value included in any one or both of the concentration data before the start of treatment and the concentration data after the start of treatment. At step S12, the risk of developing an adverse effect with treatment with the immune checkpoint inhibitor in the subject to be evaluated may be evaluated using the concentration value included in the concentration data before the start of treatment.

As described above, in the first embodiment, at step S11, the concentration data of the subject to be evaluated is obtained, and at step S12, the pharmacological action of the immune checkpoint inhibitor in the subject to be evaluated is evaluated using the concentration value included in the concentration data of the subject to be evaluated obtained at step S11 (in short, information for evaluating the pharmacological action of the immune checkpoint inhibitor in the subject to be evaluated is obtained). This method can provide highly reliable information helpful in identifying individual differences in pharmacological action of the immune checkpoint inhibitor. In particular, when only the concentration data before the start of treatment is used at step S12, the evaluation result obtained in the present embodiment can be utilized as reference information for deciding a treatment method. When the concentration data after the start of treatment or after treatment is used at step S12, the evaluation result obtained in the present embodiment can be utilized to determine whether to continue treatment with the immune checkpoint inhibitor or can be utilized as reference information for deciding another treatment method.

It may be determined that the concentration value (which may be the ratio or the difference) of at least one metabolite among the 21 kinds of amino acids and the 15 kinds of amino acid-related metabolites reflects the pharmacological action of the immune checkpoint inhibitor in the subject to be evaluated. The concentration value (which may be the ratio or the difference) may be converted using, for example, the methods listed below, and it may be determined that the converted value reflects the pharmacological action of the immune checkpoint inhibitor in the subject to be evaluated. In other words, the concentration value or the converted value itself may be treated as the evaluation result on the pharmacological action of the immune checkpoint inhibitor in the subject to be evaluated.

The concentration value may be converted such that a possible range of the concentration value falls within a predetermined range (for example, the range from 0.0 to 1.0, the range from 0.0 to 10.0, the range from 0.0 to 100.0, or the range from −10.0 to 10.0), for example, by addition, subtraction, multiplication, and division of any given value with the concentration value, by conversion of the concentration value by a predetermined conversion method (for example, exponential transformation, logarithm transformation, angular transformation, square root transformation, probit transformation, reciprocal transformation, Box-Cox transformation, or power transformation), or by performing a combination of these computations on the concentration value. For example, a value of an exponential function with the concentration value as an exponent and Napier constant as the base (specifically, a value of p/(1−p) where a natural logarithm ln(p/(1−p)) is equal to the concentration value when the probability p that the prognosis of treatment with the immune checkpoint inhibitor is not good or the risk of developing an adverse effect with the treatment is high is defined) may be further calculated, and a value (specifically, a value of the probability p) may be further calculated by dividing the calculated value of the exponential function by the sum of 1 and the value of the exponential function.

The concentration value may be converted such that the converted value is a particular value when a particular condition is met. For example, the concentration value may be converted such that the converted value is 5.0 when the specificity is 80% and the converted value is 8.0 when the specificity is 95%.

After normally distributing the concentration distribution for each amino acid and for each amino acid-related metabolite, the concentration distribution may be standardized such that the mean is 50 and the standard deviation is 10.

These conversions may be performed by gender or age.

The concentration value in the present description may be the concentration value itself or may be the converted value of the concentration value.

Positional information about a position of a predetermined mark on a predetermined scale visually presented on a display device such as a monitor or a physical medium such as paper may be generated using the concentration value (which may be the ratio or the difference) of at least one metabolite among the 21 kinds of amino acids and the 15 kinds of amino acid-related metabolites or, if the concentration value is converted, the converted value, and it may be determined that the generated positional information reflects the pharmacological action of the immune checkpoint inhibitor in the subject to be evaluated. The predetermined scale is for evaluating the pharmacological action of the immune checkpoint inhibitor in the subject to be evaluated and is, for example, a graduated scale at least marked with graduations corresponding to the upper limit value and the lower limit value in “a possible range of the concentration value or the converted value” or “part of the range”. The predetermined mark corresponds to the concentration value or the converted value and is, for example, a circle sign or a star sign.

If the concentration value (which may be the ratio or the difference) of at least one metabolite among the 21 kinds of amino acids and the 15 kinds of amino acid-related metabolites is lower than a predetermined value (e.g., mean±1SD, 2SD, 3SD, N quantile, N percentile, or a cutoff value the clinical significance of which is recognized) or is equal to or lower than the predetermined value, or the concentration value is equal to or higher than the predetermined value or is higher than the predetermined value, the pharmacological action of the immune checkpoint inhibitor in the subject to be evaluated may be evaluated. In this case, instead of the concentration value itself, a concentration standard score (a value obtained by normally distributing the concentration distribution by gender and then standardizing the concentration value with a mean of 50 and a standard deviation of 10 for each amino acid and each amino acid-related metabolite) may be used. For example, if the concentration standard score is lower than the mean−2SD (when the concentration standard score<30) or if the concentration standard score is higher than the mean+2SD (when the concentration standard score>70), the pharmacological action of the immune checkpoint inhibitor in the subject to be evaluated may be evaluated.

The pharmacological action of the immune checkpoint inhibitor in the subject to be evaluated may be discriminated by calculating a value of a formula using the concentration value (which may be the ratio or the difference) of at least one metabolite among the 21 kinds of amino acids and the 15 kinds of amino acid-related metabolites and the formula including an explanatory variable to be substituted with the concentration value (which may be the ratio or the difference).

It may be determined that the calculated value of the formula reflects the pharmacological action of the immune checkpoint inhibitor in the subject to be evaluated. The value of the formula may be converted using, for example, the methods listed below, and it may be determined that the converted value reflects the pharmacological action of the immune checkpoint inhibitor in the subject to be evaluated. In other words, the value of the formula or the converted value itself may be treated as the evaluation result on the pharmacological action of the immune checkpoint inhibitor in the subject to be evaluated.

The value of the formula may be converted such that a possible range of the value of the formula falls within a predetermined range (for example, the range from 0.0 to 1.0, the range from 0.0 to 10.0, the range from 0.0 to 100.0, or the range from −10.0 to 10.0), for example, by addition, subtraction, multiplication, and division of any given value with the value of the formula, by conversion of the value of the formula by a predetermined conversion method (for example, exponential transformation, logarithm transformation, angular transformation, square root transformation, probit transformation, reciprocal transformation, Box-Cox transformation, or power transformation), or by performing a combination of these computations on the value of the formula. For example, a value of an exponential function with the value of the formula as an exponent and Napier constant as the base (specifically, a value of p/(1−p) where a natural logarithm ln(p/(1−p)) is equal to the value of the formula when the probability p that the prognosis of treatment with the immune checkpoint inhibitor is not good or the risk of developing an adverse effect with the treatment is high is defined) may be further calculated, and a value (specifically, a value of the probability p) may be further calculated by dividing the calculated value of the exponential function by the sum of 1 and the value of the exponential function.

The value of the formula may be converted such that the converted value is a particular value when a particular condition is met. For example, the value of the formula may be converted such that the converted value is 5.0 when the specificity is 80% and the converted value is 8.0 when the specificity is 95%.

The value of the formula may be standardized such that the mean is 50 and the standard deviation is 10.

These conversions may be performed by gender or age.

The value of the formula in the present description may be the value of the formula itself or may be the converted value of the value of the formula.

Positional information about a position of a predetermined mark on a predetermined scale visually presented on a display device such as a monitor or a physical medium such as paper may be generated using the value of the formula or, if the value of the formula is converted, the converted value, and it may be determined that the generated positional information reflects the pharmacological action of the immune checkpoint inhibitor in the subject to be evaluated. The predetermined scale is for evaluating the pharmacological action of the immune checkpoint inhibitor in the subject to be evaluated and is, for example, a graduated scale at least marked with graduations corresponding to the upper limit value and the lower limit value in “a possible range of the value of the formula or the converted value” or “part of the range”. The predetermined mark corresponds to the value of the formula or the converted value and is, for example, a circle sign or a star sign.

The pharmacological action of the immune checkpoint inhibitor in the subject to be evaluated may be qualitatively evaluated. Specifically, the subject to be evaluated may be classified into any one of a plurality of categories defined at least in consideration of the prognosis of treatment with the immune checkpoint inhibitor or the risk of developing an adverse effect with the treatment, using “the concentration value (which may be the ratio or the difference) of at least one metabolite among the 21 kinds of amino acids and the 15 kinds of amino acid-related metabolites, and preset one or more thresholds” or “the concentration value (which may be the ratio or the difference) of at least one metabolite among the 21 kinds of amino acids and the 15 kinds of amino acid-related metabolites, a formula including an explanatory variable to be substituted with the concentration value (which may be the ratio or the difference), and preset one or more thresholds”. The categories may include a category to which a subject with poor prognosis of the treatment or with a high risk of developing an adverse effect with the treatment belongs, a category to which a subject with good prognosis of the treatment or with a low risk of developing an adverse effect with the treatment belongs, and a category to which a subject with prognosis of the treatment being intermediate between good and poor or with an intermediate risk of developing an adverse effect with the treatment belongs. The categories may include a category to which a subject with poor prognosis of the treatment or with a high risk of developing an adverse effect with the treatment belongs and a category to which a subject with good prognosis of the treatment or with a low risk of developing an adverse effect with the treatment belongs. The concentration value (which may be the ratio or the difference) or the value of the formula may be converted by a predetermined method, and the subject to be evaluated may be classified into any one of the categories using the converted value.

As for the formula used for the evaluation, the form of the formula is not specifically designated, however, for example, may be the following forms.
linear model such as multiple regression equation, linear discriminant, principal component analysis, and canonical discriminant analysis that are based on the least-squares method;
generalized linear model such as logistic regression and Cox regression that are based on the maximum likelihood method;
generalized linear mixed model considering random effects due to individual differences, facility differences, and other factors in addition to the generalized linear model
expression generated by cluster analysis such as the K-means method and hierarchical cluster analysis;
expression generated on the basis of the Bayesian statistics such as the Markov chain Monte Carlo (MCMC), the Bayesian network, and the hierarchical Bayesian method;
expression generated by class classification such as support vector machine and decision tree;
expression generated by a method that does not belong to the above-cited categories such as fractional expression; and
expression represented as, for example, the summation of expressions of different forms

The formula used for the evaluation may be prepared by a method described in WO 2004/052191 that is an international application filed by the present applicant or by a method described in WO 2006/098192 that is an international application filed by the present applicant. Any formulae obtained by these methods can be preferably used in the evaluation of the pharmacological action of the immune checkpoint inhibitor, regardless of the units of concentrations of amino acids or amino acid-related metabolites in the concentration data as input data.

In the multiple regression equation, the multiple logistic regression equation, and the canonical discriminant function, a coefficient and a constant term are added to each explanatory variable, and the coefficient and the constant term may be preferably real numbers, more preferably values in the range of 99% confidence interval for the coefficient and the constant term obtained from data for the various kinds of classifications described above, more preferably values in the range of 95% confidence interval for the coefficient and the constant term obtained from data for the various kinds of classifications described above. The value of each coefficient and the confidence interval thereof may be those multiplied by a real number, and the value of the constant term and the confidence interval thereof may be those having an arbitrary actual constant added or subtracted or those multiplied or divided by an arbitrary actual constant. When an expression such as the logistic regression, the linear discriminant, and the multiple regression equation is used for the evaluation, a linear transformation of the expression (addition of a constant and multiplication by a constant) and a monotonic increasing (decreasing) transformation (for example, a logit transformation) of the expression do not alter evaluation performance and thus evaluation performance after transformation is equivalent to that before transformation. Therefore, the expression includes an expression that is subjected to the linear transformation and the monotonic increasing (decreasing) transformation.

In the fractional expression, the numerator of the fractional expression is expressed by the sum of the explanatory variables A, B, C etc. and the denominator of the fractional expression is expressed by the sum of the explanatory variables a, b, c etc. The fractional expression also includes the sum of the fractional expressions α, β, γ etc. (for example, α+β) having such constitution. The fractional expression also includes divided fractional expressions. The explanatory variables used in the numerator or denominator may have suitable coefficients respectively. The explanatory variables used in the numerator or denominator may appear repeatedly. Each fractional expression may have a suitable coefficient. A value of a coefficient for each explanatory variable and a value for a constant term may be any real numbers. In a fractional expression and the one in which explanatory variables in the numerator and explanatory variables in the denominator in the fractional expression are switched with each other, the positive and negative signs are generally reversed in correlation with objective explanatory variables, but because their correlation is maintained, the evaluation performance can be assumed to be equivalent. The fractional expression therefore also includes the one in which explanatory variables in the numerator and explanatory variables in the denominator in the fractional expression are switched with each other.

When the pharmacological action of the immune checkpoint inhibitor is evaluated, a value related to other biological information (for example, values listed below) may further be used in addition to the concentration value of at least one metabolite among the 21 kinds of amino acids and the 15 kinds of amino acid-related metabolites. The formula used for the evaluation may additionally include one or more explanatory variables to be substituted with a value related to the other biological information (for example, values listed below) in addition to the explanatory variable to be substituted with the concentration value of at least one metabolite among the 21 kinds of amino acids and the 15 kinds of amino acid-related metabolites.

1. Concentration values of metabolites (carbohydrates, lipids, etc.) in blood, other than amino acids and amino acid-related metabolites, proteins, peptides, minerals, hormones, and the like
2. Blood test values such as tumor markers, albumin, total protein, triglycerides, HbA1c, LDL cholesterol, HDL cholesterol, amylase, total bilirubin, and uric acid
3. Immune-related test values such as blood cytokines, immunocompetent cell count, cytokines in immunocompetent cells, and delayed type hyperreactivity (DTH)
4. Values obtained from image information from ultrasound echo, upper and lower endoscopy, X-ray, CT, MRI, and the like
5. Values related to biological indicators such as age, height, weight, BMI, blood pressure, gender, smoking information, diet information, drinking information, exercise information, stress information, sleep information, family medical history information, and disease history information (diabetes, pancreatitis, etc.)
6. Values obtained from multilayer omics analysis information, information on cancer gene mutations, information on microsatellite instability, information on cancer-derived antigens and antibodies, or information on the expression of molecules such as PD-1 and PD-L1

### Second Embodiment

### 2-1. Outline of the Second Embodiment

Here, outlines of the second embodiment will be described in detail with reference to FIG. 2. FIG. 2 is a principle configurational diagram showing a basic principle of the second embodiment. In the description of the present second embodiment, description duplicating that of the first embodiment is sometimes omitted. In particular, herein, when the pharmacological action of the immune checkpoint inhibitor is evaluated, a case of using the value of the formula or the converted value thereof is described as one example. However, for example, the concentration value, the ratio or the difference of the concentration values, or the converted value thereof (for example, a concentration standard score) may be used.

A control device evaluates the pharmacological action of the immune checkpoint inhibitor in the subject to be evaluated by calculating the value of the formula using (i) the concentration value included the previously obtained concentration data on the concentration value of at least one metabolite among the 21 kinds of amino acids and the 15 kinds of amino acid-related metabolites in blood taken from the subject to be evaluated (for example, an individual such as an animal or a human) that may be treated with the immune checkpoint inhibitor and (ii) the formula previously stored in a memory device, including the explanatory variable to be substituted with the concentration value (step S21). When both of the concentration data before the start of treatment and the concentration data after the start of treatment are used at step S21, for example, the control device may evaluate the pharmacological action of the immune checkpoint inhibitor in the subject to be evaluated by calculating the ratio or the difference between the concentration value before the start of treatment and the concentration value after the start of treatment and calculating the value of the formula by substituting the calculated ratio or difference for the explanatory variable. This apparatus can provide highly reliable information helpful in identifying individual differences in pharmacological action of the immune checkpoint inhibitor.

The formula used at step S21 may be generated based on the formula-preparing processing (step 1 to step 4) described below. Here, the summary of the formula-preparing processing is described. The processing described below is merely one example, and the method of preparing the formula is not limited thereto.

First, the control device prepares a candidate formula (e.g., y=a₁x₁+a₂x₂+ . . . +aₙxₙ, y: index data, xᵢ: concentration data, aᵢ: constant, i=1, 2, . . . , n) based on a predetermined formula-preparing method from index state information (data such as defective and outliers may be removed from the index state information in advance) previously stored in the memory device (step 1). The index state information includes concentration data (for example, concentration data of amino acids and amino acid-related metabolites before the start of treatment, concentration data of amino acids and amino acid-related metabolites after the start of treatment, or concentration data on the amount of change of amino acids and amino acid-related metabolites between before the start of treatment and after the start of treatment) and index data on the prognosis of treatment with the immune checkpoint inhibitor or the risk of developing an adverse effect with the treatment (for example, binary data on poor or good prognosis or binary data on whether an adverse effect is developed).

In step 1, a plurality of the candidate formulae may be prepared from the index state information by using a plurality of the different formula-preparing methods (including those for multivariate analysis such as the principal component analysis, the discriminant analysis, the support vector machine, the multiple regression analysis, the Cox regression analysis, the logistic regression analysis, the K-means method, the cluster analysis, and the decision tree). Specifically, a plurality of groups of the candidate formulae may be prepared simultaneously and concurrently by using a plurality of different algorithms with the index state information which is multivariate data composed of the index data and the concentration data obtained by analyzing blood taken before the start of treatment and/or after the start of treatment from a large number of patients that may be treated with the immune checkpoint inhibitor. For example, the two different candidate formulae may be formed by performing the discriminant analysis and the logistic regression analysis simultaneously with the different algorithms. Alternatively, the candidate formula may be formed by converting the index state information with the candidate formula prepared by performing the principal component analysis and then performing the discriminant analysis of the converted index state information. In this way, it is possible to finally prepare the most suitable formula for the evaluation.

The candidate formula prepared by the principal component analysis is a linear expression including each explanatory variable maximizing the variance of all concentration data. The candidate formula prepared by the discriminant analysis is a high-powered expression (including exponential and logarithmic expressions) including each explanatory variable minimizing the ratio of the sum of the variances in respective groups to the variance of all concentration data. The candidate formula prepared by using the support vector machine is a high-powered expression (including kernel function) including each explanatory variable maximizing the boundary between groups. The candidate formula prepared by using the multiple regression analysis is a high-powered expression including each explanatory variable minimizing the sum of the distances from all concentration data. The candidate formula prepared by using the Cox regression analysis is a linear model including a logarithmic hazard ratio, and is a linear expression including each explanatory variable with a coefficient thereof maximizing the likelihood of the linear model. The candidate formula prepared by using the logistic regression analysis is a linear model expressing logarithmic odds of probability, and a linear expression including each explanatory variable maximizing the likelihood of the probability. The K-means method is a method of searching k pieces of neighboring concentration data in various groups, designating the group containing the greatest number of the neighboring points as its data-belonging group, and selecting the explanatory variable that makes the group to which input concentration data belong agree well with the designated group. The cluster analysis is a method of clustering (grouping) the points closest in entire concentration data. The decision tree is a method of ordering explanatory variables and predicting the group of concentration data from the pattern possibly held by the higher-ordered explanatory variable.

Returning to the description of the formula-preparing processing, the control device verifies (mutually verifies) the candidate formula prepared in step 1 based on a particular verifying method (step 2). The verification of the candidate formula is performed on each other to each candidate formula prepared in step 1. In step 2, at least one of discrimination rate, sensitivity, specificity, information criterion (Akaike information criterion (AIC), Bayesian information criterion (BIC)), ROC_AUC (area under the curve in a receiver operating characteristic curve), C-index (Concordance index), and the like of the candidate formula may be verified by at least one of bootstrap method, holdout method, N-fold method, leave-one-out method, and the like. In this way, it is possible to prepare the candidate formula higher in predictability or reliability, by taking the index state information and the evaluation condition into consideration.

The discrimination rate is a rate in which a subject to be evaluated whose true state is negative (for example, a subject with good prognosis of the treatment or without developing an adverse effect with the treatment) is correctly evaluated as being negative by the evaluation method according to the present embodiment and a subject to be evaluated whose true state is positive (for example, a subject with poor prognosis of the treatment or with developing an adverse effect with the treatment) is correctly evaluated as being positive by the evaluation method according to the present embodiment. The sensitivity is a rate in which a subject to be evaluated whose true state is positive is correctly evaluated as being positive by the evaluation method according to the present embodiment. The specificity is a rate in which a subject to be evaluated whose true state is negative is correctly evaluated as being negative by the evaluation method according to the present embodiment. The Akaike information criterion (AIC) is a criterion representing how observation data agrees with a statistical model, for example, in the regression analysis, and it is determined that the model in which the value defined by “−2×(maximum log-likelihood of statistical model)+2×(the number of free parameters of statistical model)” is smallest is the best. The Bayesian information criterion (BIC) is a model selection criterion derived based on the concept of Bayesian statistics. A model with the smallest value defined by “−2×(maximum log-likelihood of statistical model)+(the number of free parameters of statistical model)×ln (sample size)” (a model with a fewer parameters) is determined to be the best. ROC_AUC is defined as the area under the receiver operating characteristics curve (ROC) created by plotting (x, y)=(1-specificity, sensitivity) on two-dimensional coordinates. The value of ROC_AUC is 1 in perfect discrimination, and the closer this value is to 1, the higher the discriminative characteristic. The C-index is an index of the accuracy of prognosis prediction proposed by Harrell et al. and is a non-parametric index that indicates how much the magnitudes of the probability of event occurrence predicted by a model and the probability of the actual event occurrence match. The predictability is the average of discrimination rates, sensitivities, or specificities obtained by repeating the validation of the candidate formula. The robustness refers to the variance of discrimination rates, sensitivities, or specificities obtained by repeating the validation of the candidate formula.

Returning to the description of the formula-preparing processing, the control device selects a combination of the concentration data contained in the index state information used in preparing the candidate formula, by selecting an explanatory variable of the candidate formula based on a predetermined explanatory variable-selecting method (step 3). In step 3, the selection of the explanatory variable may be performed on each candidate formula prepared in step 1. In this way, it is possible to select the explanatory variable of the candidate formula properly. The step 1 is executed once again by using the index state information including the concentration data selected in step 3. In step 3, the explanatory variable of the candidate formula may be selected based on at least one of stepwise method, best path method, local search method, and genetic algorithm from the verification result obtained in step 2. The best path method is a method of selecting an explanatory variable by optimizing an evaluation index of the candidate formula while eliminating the explanatory variables contained in the candidate formula one by one.

Returning to the description of the formula-preparing processing, the control device prepares the formula used for the evaluation by repeatedly performing steps 1, 2 and 3, and based on the verification results thus accumulated, selecting the candidate formula used for the evaluation from the candidate formulae (step 4). In the selection of the candidate formula, there are cases where the optimum formula is selected from the candidate formulae prepared in the same formula-preparing method or the optimum formula is selected from all candidate formulae.

As described above, in the formula-preparing processing, the processing for the preparation of the candidate formulae, the verification of the candidate formulae, and the selection of the explanatory variables in the candidate formulae are performed based on the index state information in a series of operations in a systematized manner, whereby the formula most appropriate for evaluating the pharmacological action of the immune checkpoint inhibitor can be prepared. In other words, in the formula-preparing processing, the concentration value of at least one metabolite among the 21 kinds of amino acids and the 15 kinds of amino acid-related metabolites is used in multivariate statistical analysis, and for selecting the optimum and robust combination of the explanatory variables, the explanatory variable-selecting method is combined with cross-validation to extract the formula having high evaluation performance.

### 2-2. System Configuration

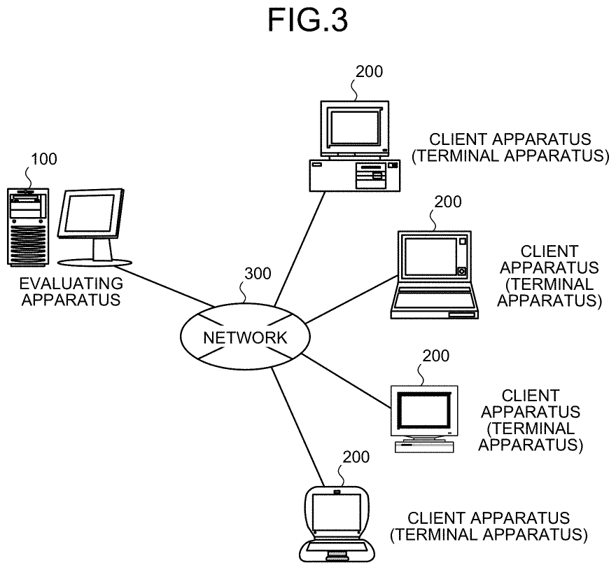

Hereinafter, the configuration of the evaluating system according to the second embodiment (hereinafter referred to sometimes as the present system) will be described with reference to FIGS. 3 to 13. This system is merely one example, and the present invention is not limited thereto. In particular, herein, when the pharmacological action of the immune checkpoint inhibitor is evaluated, a case of using the value of the formula or the converted value thereof is described as one example. However, for example, the concentration value, the ratio or the difference of the concentration values, or the converted value thereof (for example, a concentration standard score) may be used.

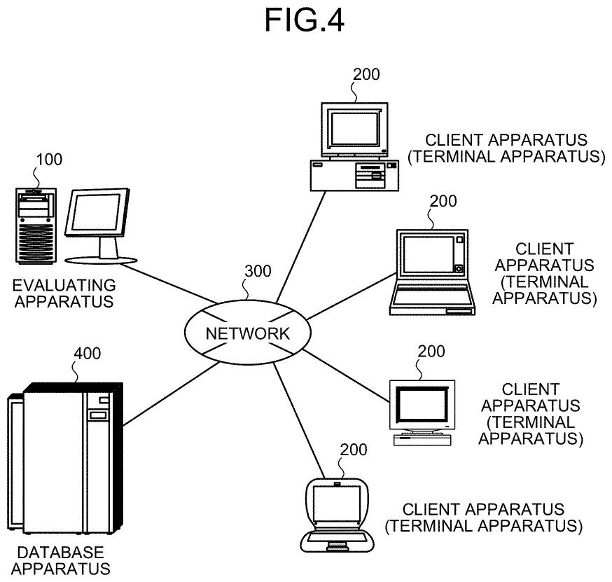

First, an entire configuration of the present system will be described with reference to FIGS. 3 and 4. FIG. 3 is a diagram showing an example of the entire configuration of the present system. FIG. 4 is a diagram showing another example of the entire configuration of the present system. As shown in FIG. 3, the present system is constituted in which the evaluating apparatus 100 that evaluates the pharmacological action of the immune checkpoint inhibitor in the individual as the subject to be evaluated and the client apparatus 200 (corresponding to the terminal apparatus of the present invention) that provides the concentration data of the individual on the concentration value of at least one metabolite among the 21 kinds of amino acids and the 15 kinds of amino acid-related metabolites in blood, are communicatively connected to each other via a network 300.

In the present system, the client apparatus 200 that provides data for use in the evaluation and the client apparatus 200 that receives the evaluation result may be different. In the present system as shown in FIG. 4, in addition to the evaluating apparatus 100 and the client apparatus 200, the database apparatus 400 storing, for example, the index state information used in preparing the formula in the evaluating apparatus 100 and the formula used for the evaluation, may be communicatively connected via the network 300.

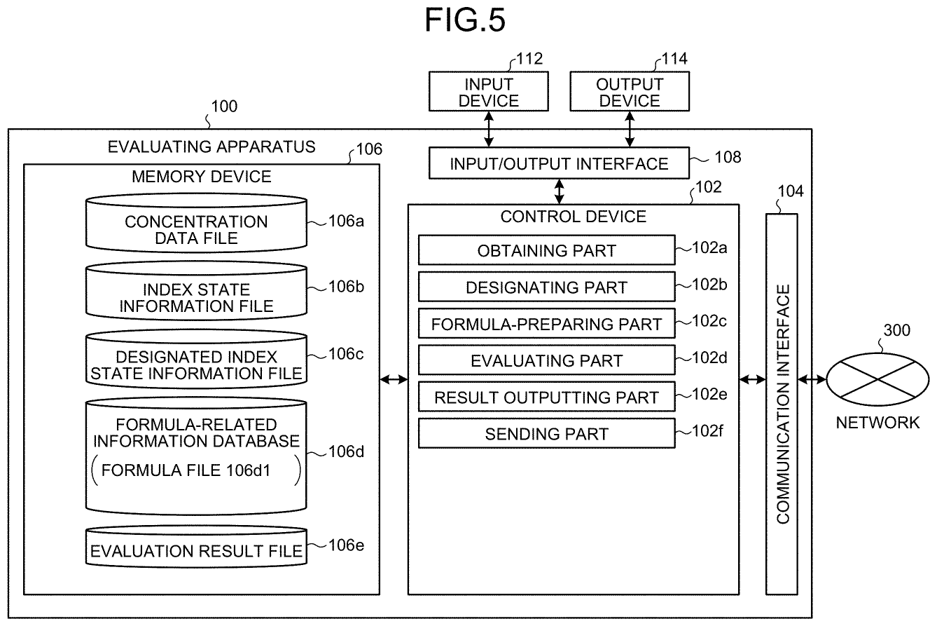

Now, the configuration of the evaluating apparatus 100 in the present system will be described with reference to FIGS. 5 to 11. FIG. 5 is a block diagram showing an example of the configuration of the evaluating apparatus 100 in the present system, showing conceptually only the region relevant to the present invention.

The evaluating apparatus 100 includes: (i) a control device 102, such as CPU (Central Processing Unit), that integrally controls the evaluating apparatus; (ii) a communication interface 104 that connects the evaluating apparatus to the network 300 communicatively via communication apparatuses such as a router and wired or wireless communication lines such as a private line; (iii) a memory device 106 that stores various databases, tables, files and others; and (iv) an input/output interface 108 connected to an input device 112 and an output device 114, and these parts are connected to each other communicatively via any communication channel. The evaluating apparatus 100 may be present together with various analyzers (e.g., an analyzer for amino acids and amino acid-related metabolites) in a same housing. For example, the evaluating apparatus 100 may be a compact analyzing device including components (hardware and software) that calculate (measure) the concentration value of at least one metabolite among the 21 kinds of amino acids and the 15 kinds of amino acid-related metabolites in blood and output (e.g., print or display on a monitor) the calculated value, wherein the compact analyzing device is characterized by further including the evaluating part 102d described later, and using the components to output results obtained by the evaluating part 102d.

The communication interface 104 allows communication between the evaluating apparatus 100 and the network 300 (or a communication apparatus such as a router). Thus, the communication interface 104 has a function to communicate data via a communication line with other terminals.

The input/output interface 108 is connected to the input device 112 and the output device 114. A monitor (including a home television), a speaker, or a printer may be used as the output device 114 (hereinafter, the output device 114 may be described as the monitor 114). A keyboard, a mouse, a microphone, or a monitor functioning as a pointing device together with a mouse may be used as the input device 112.

The memory device 106 is a storage means, and examples thereof include a memory apparatus such as RAM (Random Access Memory) and ROM (Read Only Memory), a fixed disk drive such as a hard disk, a flexible disk, and an optical disk. The memory device 106 stores computer programs giving instructions to the CPU for various processings, together with OS (Operating System). As shown in the figure, the memory device 106 stores the concentration data file 106a, the index state information file 106b, the designated index state information file 106c, a formula-related information database 106d, and the evaluation result file 106e.

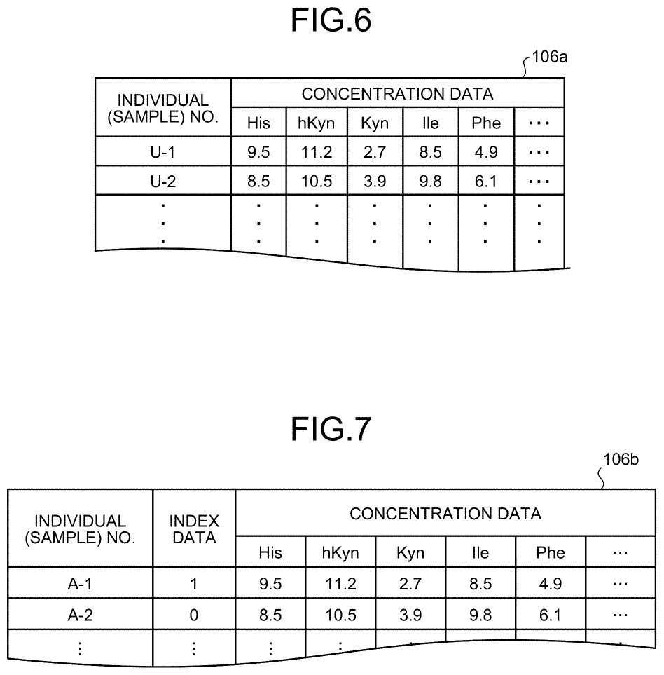

The concentration data file 106a stores the concentration data on the concentration value of at least one metabolite among the 21 kinds of amino acids and the 15 kinds of amino acid-related metabolites in blood (for example, any one or both of the concentration data before the start of treatment and the concentration data after the start of treatment). FIG. 6 is a chart showing an example of information stored in the concentration data file 106a. As shown in FIG. 6, the information stored in the concentration data file 106a includes an individual number for uniquely identifying the individual (sample) as the subject to be evaluated and the concentration data that are correlated to one another. In FIG. 6, the concentration data is assumed to be numerical values, i.e., on a continuous scale, but the concentration data may be expressed on a nominal scale or an ordinal scale. In the case of the nominal or ordinal scale, any number may be allocated to each state for analysis. The concentration data may be combined with a value related to the other biological information (see above).

Returning to FIG. 5, the index state information file 106b stores the index state information used in preparing the formula. FIG. 7 is a chart showing an example of information stored in the index state information file 106b. As shown in FIG. 7, the information stored in the index state information file 106b includes the individual number, the index data on the prognosis of treatment with the immune checkpoint inhibitor or the risk of developing an adverse effect with the treatment, and the concentration data that are correlated to one another. In FIG. 7, the index data and the concentration data are assumed to be numerical values, i.e., on a continuous scale, but the index data and the concentration data may be expressed on a nominal scale or an ordinal scale. In the case of the nominal or ordinal scale, any number may be allocated to each state for analysis.

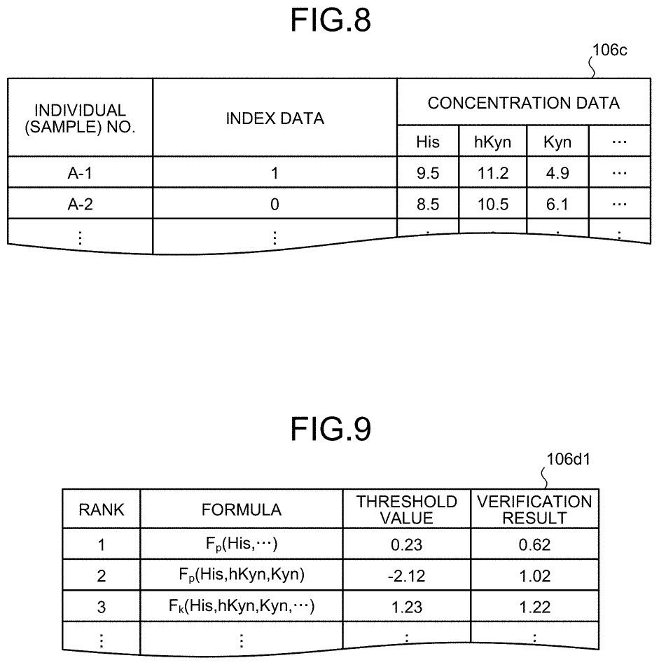

Returning to FIG. 5, the designated index state information file 106c stores the index state information designated in a designating part 102b described below. FIG. 8 is a chart showing an example of information stored in the designated index state information file 106c. As shown in FIG. 8, the information stored in the designated index state information file 106c includes the individual number, the designated index data, and the designated concentration data that are correlated to one another.

Returning to FIG. 5, the formula-related information database 106d is composed of the formula file 106d1 storing the formula prepared in a formula-preparing part 102c described below. The formula file 106d1 stores the formulae used for the evaluation. FIG. 9 is a chart showing an example of information stored in the formula file 106d1. As shown in FIG. 9, the information stored in the formula file 106d1 includes a rank, the formula (e.g., Fₚ (His, . . . ), Fₚ (His, hKyn, Kyn), Fₖ (His, hKyn, Kyn, . . . ) in FIG. 9), a threshold corresponding to each formula-preparing method, and the verification result of each formula (e.g., the value of each formula) that are correlated to one another.

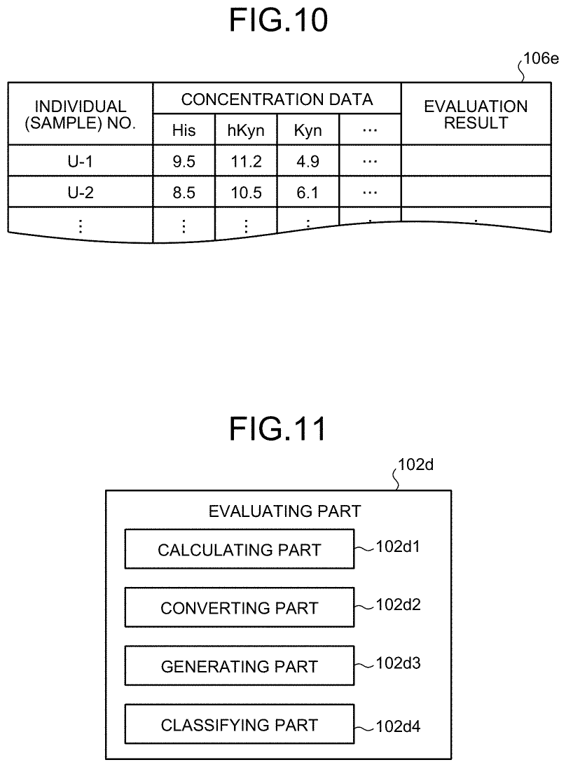

Returning to FIG. 5, the evaluation result file 106e stores the evaluation results obtained in the evaluating part 102d described below. FIG. 10 is a chart showing an example of information stored in the evaluation result file 106e. The information stored in the evaluation result file 106e includes the individual number for uniquely identifying the individual (sample) as the subject to be evaluated, the previously obtained concentration data of the individual, and the evaluation result on the pharmacological action of the immune checkpoint inhibitor (the prognosis of treatment or the risk of developing an adverse effect), that are correlated to one another. The evaluation result includes, for example, the value of the formula calculated by a calculating part 102d1 described below, the converted value of the value of the formula obtained by a converting part 102d2 described below, the positional information generated by a generating part 102d3 described below, or the classification result obtained by a classifying part 102d4 described below.

Returning to FIG. 5, the control device 102 has an internal memory storing, for example, control programs such as OS (Operating System), programs for various processing procedures, and other needed data, and performs various information processings according to these programs. As shown in the figure, the control device 102 includes mainly an obtaining part 102a, the designating part 102b, the formula-preparing part 102c, the evaluating part 102d, a result outputting part 102e, and a sending part 102f. The control device 102 performs data processings such as removal of data including defective, removal of data including many outliers, and removal of explanatory variables for the defective-including data in the index state information transmitted from the database apparatus 400 and in the concentration data transmitted from the client apparatus 200.

The obtaining part 102a obtains information (specifically, concentration data, index state information, formula, and the like). For example, the obtaining part 102a may receive information (specifically, concentration data, index state information, formula, and the like) transmitted from the client apparatus 200 or the database apparatus 400 via the network 300 to obtain information. The obtaining part 102a may receive data for use in evaluation transmitted from a client apparatus 200 different from the client apparatus 200 to which the evaluation result is transmitted. When the evaluating apparatus 100 includes a mechanism (including hardware and software) for reading information recorded on a recording medium, the obtaining part 102a may obtain information by reading information recorded on a recording medium (specifically, concentration data, index state information, formula, and the like) through the mechanism. The specifying part 102b specifies target index data and concentration data in creating a formula.

The formula-preparing part 102c generates the formula based on the index state information obtained in the obtaining part 102a or the index state information designated in the designating part 102b. If the formulae are stored previously in a predetermined region of the memory device 106, the formula-preparing part 102c may generate the formula by selecting the desired formula out of the memory device 106. Alternatively, the formula-preparing part 102c may generate the formula by selecting and downloading the desired formula from another computer apparatus (e.g., the database apparatus 400) in which the formulae are previously stored.

The evaluating part 102d evaluates the pharmacological action of the immune checkpoint inhibitor in the individual by calculating the value of the formula using (i) the previously obtained formula (for example, the formula prepared by the formula-preparing part 102c or the formula obtained by the obtaining part 102a) and (ii) the concentration value included in the concentration data of the individual obtained by the obtaining part 102a. The evaluating part 102d may evaluate the pharmacological action of the immune checkpoint inhibitor in the individual using (i) the concentration value of at least one metabolite among the 21 kinds of amino acids and the 15 kinds of amino acid-related metabolites, (ii) the ratio or the difference of concentration values, or (iii) the converted value thereof (for example, the concentration standard score).

Hereinafter, a configuration of the evaluating part 102d will be described with reference to FIG. 11. FIG. 11 is a block diagram showing the configuration of the evaluating part 102d, and only a part in the configuration related to the present invention is shown conceptually. The evaluating part 102d includes the calculating part 102d1, the converting part 102d2, the generating part 102d3, and the classifying part 102d4, additionally.

The calculating part 102d1 calculates the value of the formula using (i) the concentration value (which may be the ratio or the difference) of at least one metabolite among the 21 kinds of amino acids and the 15 kinds of amino acid-related metabolites and (ii) the formula including the explanatory variable to be substituted with the concentration value. The evaluating part 102d may store the value of the formula calculated by the calculating part 102d1 as the evaluation result in a predetermined region of the evaluation result file 106e.

The converting part 102d2 converts the value of the formula calculated by the calculating part 102d1, for example, by the conversion method described above. The evaluating part 102d may store the converted value by the converting part 102d2 as the evaluation result in a predetermined region of the evaluation result file 106e. The converting part 102d2 may convert the concentration value included in the concentration data, or the ratio or the difference of the concentration values, for example, by the conversion method described above.

The generating part 102d3 generates the positional information about the position of the predetermined mark on the predetermined scale visually presented on the display device such as a monitor or the physical medium such as paper, using the value of the formula calculated by the calculating part 102d1 or the converted value by the converting part 102d2 (the concentration value, the ratio or the difference of the concentration values, or the converted value thereof may be used as well). The evaluating part 102d may store the positional information generated by the generating part 102d3 as the evaluation result in a predetermined region of the evaluation result file 106e.

The classifying part 102d4 classifies the individual into any one of the categories defined at least in consideration of the prognosis of treatment with the immune checkpoint inhibitor or the risk of developing an adverse effect with the treatment, using the value of the formula calculated by the calculating part 102d1 or the converted value by the converting part 102d2 (the concentration value, the ratio or the difference of the concentration values, or the converted value thereof may be used as well).

The result outputting part 102e outputs, into the output device 114, for example, the processing results in each processing part in the control device 102 (including the evaluation results obtained by the evaluating part 102d).

The sending part 102f transmits the evaluation results to the client apparatus 200 that is a sender of the concentration data of the individual, and transmits the formulae prepared in the evaluating apparatus 100 and the evaluation results to the database apparatus 400. The sending part 102f may transmit the evaluation result to a client apparatus 200 different from the client apparatus 200 that from which data for use in evaluation is transmitted.

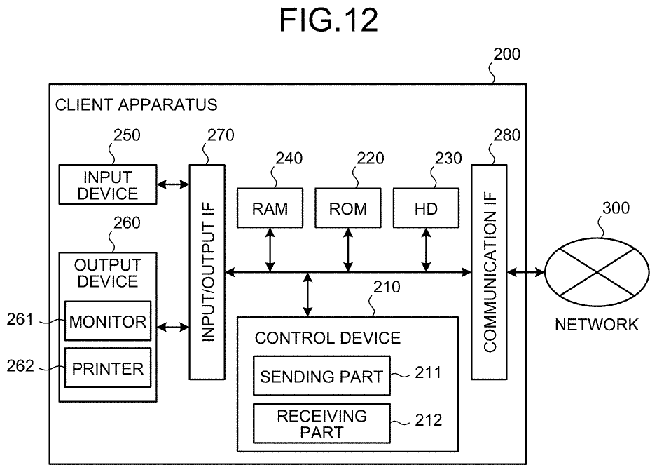

Hereinafter, a configuration of the client apparatus 200 in the present system will be described with reference to FIG. 12. FIG. 12 is a block diagram showing an example of the configuration of the client apparatus 200 in the present system, and only the part in the configuration relevant to the present invention is shown conceptually.

The client apparatus 200 includes a control device 210, ROM 220, HD (Hard Disk) 230, RAM 240, an input device 250, an output device 260, an input/output IF 270, and a communication IF 280 that are connected communicatively to one another through a communication channel. The client apparatus 200 may be realized based on an information processing apparatus (for example, an information processing terminal such as a known personal computer, a workstation, a family computer, Internet TV (Television), PHS (Personal Handyphone System) terminal, a mobile phone terminal, a mobile unit communication terminal, or PDA (Personal Digital Assistants)) connected as needed with peripheral devices such as a printer, a monitor, and an image scanner.

The input device 250 is, for example, a keyboard, a mouse, or a microphone. The monitor 261 described below also functions as a pointing device together with a mouse. The output device 260 is an output means for outputting information received via the communication IF 280, and includes the monitor 261 (including home television) and a printer 262. In addition, the output device 260 may have a speaker or the like additionally. The input/output IF 270 is connected to the input device 250 and the output device 260.

The communication IF 280 connects the client apparatus 200 to the network 300 (or communication apparatus such as a router) communicatively. In other words, the client apparatus 200 is connected to the network 300 via a communication apparatus such as a modem, TA (Terminal Adapter) or a router, and a telephone line, or via a private line. In this way, the client apparatus 200 can access to the evaluating apparatus 100 by using a particular protocol.

The control device 210 has a receiving part 211 and a sending part 212. The receiving part 211 receives various kinds of information such as the evaluation results transmitted from the evaluating apparatus 100, via the communication IF 280. The sending part 212 sends various kinds of information such as the concentration data of the individual, via the communication IF 280, to the evaluating apparatus 100.

All or a part of processings of the control device 210 may be performed by CPU and programs read and executed by the CPU. Computer programs for giving instructions to the CPU and executing various processings together with the OS (Operating System) are recorded in the ROM 220 or HD 230. The computer programs, which are executed as they are loaded in the RAM 240, constitute the control device 210 with the CPU. The computer programs may be stored in application program servers connected via any network to the client apparatus 200, and the client apparatus 200 may download all or a part of them as needed. All or any part of processings of the control device 210 may be realized by hardware such as wired-logic.

The control device 210 may include an evaluating part 210a (including a calculating part 210a1, a converting part 210a2, a generating part 210a3, and a classifying part 210a4) having the same functions as the functions of the evaluating part 102d in the evaluating apparatus 100. When the control device 210 includes the evaluating part 210a, the evaluating part 210a may convert the value of the formula (the concentration value, or the ratio or the difference of the concentration values may be used as well) in the converting part 210a2, generate the positional information corresponding to the value of the formula or the converted value (the concentration value, the ratio or the difference of the concentration values, or the converted value thereof may be used as well) in the generating part 210a3, and classify the individual into any one of the categories using the value of the formula or the converted value (the concentration value, the ratio or the difference of the concentration values, or the converted value thereof may be used as well) in the classifying part 210a4, in accordance with information included in the evaluation results transmitted from the evaluating apparatus 100.

Hereinafter, the network 300 in the present system will be described with reference to FIGS. 3 and 4. The network 300 has a function to connect the evaluating apparatus 100, the client apparatuses 200, and the database apparatus 400 mutually, communicatively to one another, and is for example the Internet, an intranet, or LAN (Local Area Network (including both wired and wireless)). The network 300 may be VAN (Value Added Network), a personal computer communication network, a public telephone network (including both analog and digital), a leased line network (including both analog and digital), CATV (Community Antenna Television) network, a portable switched network or a portable packet-switched network (including IMT2000 (International Mobile Telecommunication 2000) system, GSM (registered trademark) (Global System for Mobile Communications) system, or PDC (Personal Digital Cellular)/PDC-P system), a wireless calling network, a local wireless network such as Bluetooth (registered trademark), PHS network, a satellite communication network (including CS (Communication Satellite), BS (Broadcasting Satellite), ISDB (Integrated Services Digital Broadcasting), and the like), or the like.

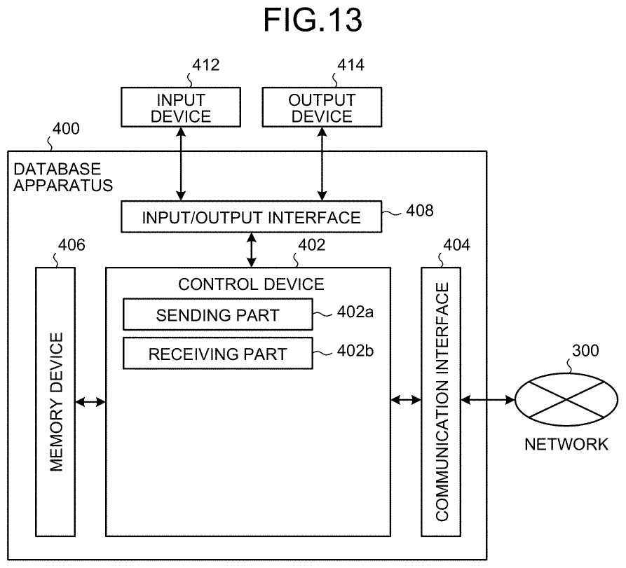

Hereinafter, the configuration of the database apparatus 400 in the present system will be described with reference to FIG. 13. FIG. 13 is a block diagram showing an example of the configuration of the database apparatus 400 in the present system, showing conceptually only the region relevant to the present invention.

The database apparatus 400 has functions to store, for example, the index state information used in preparing the formulae in the evaluating apparatus 100 or the database apparatus, the formulae prepared in the evaluating apparatus 100, and the evaluation results obtained in the evaluating apparatus 100. As shown in FIG. 13, the database apparatus 400 includes; (i) a control device 402, such as CPU, that integrally controls the database apparatus; (ii) a communication interface 404 connecting the database apparatus to the network 300 communicatively via communication apparatuses such as a router and wired or wireless communication circuits such as a private line; (iii) a memory device 406 storing various databases, tables, files (for example, files for Web pages) and others; and (iv) an input/output interface 408 connected to an input device 412 and an output device 414, and these parts are connected communicatively to each other via any communication channel.

The memory device 406 is a storage means, and, examples thereof include a memory apparatus such as RAM or ROM, a fixed disk drive such as a hard disk, a flexible disk, and an optical disk. The memory device 406 stores, for example, various programs used in various processings. The communication interface 404 allows communication between the database apparatus 400 and the network 300 (or a communication apparatus such as a router). Thus, the communication interface 404 has a function to communicate data via a communication line with other terminals. The input/output interface 408 is connected to the input device 412 and the output device 414. A monitor (including a home television), a speaker, or a printer may be used as the output device 414. A keyboard, a mouse, a microphone, or a monitor functioning as a pointing device together with a mouse may be used as the input device 412.

The control device 402 has an internal memory storing, for example, control programs such as OS (Operating System), programs for various processing procedures, and other needed data, and performs various information processings according to these programs. As shown in the figure, the control device 402 includes mainly a sending part 402a and a receiving part 402b. The sending part 402a transmits various kinds of information such as the index state information and the formulae to the evaluating apparatus 100. The receiving part 402b receives various kinds of information such as the formula and the evaluation results, transmitted from the evaluating apparatus 100.

In the present description, the evaluating apparatus 100 executes the obtainment of the concentration data, the calculation of the value of the formula, the classification of the individual into the category, and the transmission of the evaluation results, while the client apparatus 200 executes the reception of the evaluation results, described as an example. However, when the client apparatus 200 includes the evaluating unit 210a, the evaluating apparatus 100 only has to execute the calculation of the value of the formula. For example, the conversion of the value of the formula, the generation of the positional information, and the classification of the individual into the category may be appropriately shared between the evaluating apparatus 100 and the client apparatus 200.

For example, when the client apparatus 200 receives the value of the formula from the evaluating apparatus 100, the evaluating unit 210a may convert the value of the formula in the converting unit 210a2, generate the positional information corresponding to the value of the formula or the converted value in the generating unit 210a3, and classify the individual into any one of the categories using the value of the formula or the converted value in the classifying unit 210a4.

When the client apparatus 200 receives the converted value from the evaluating apparatus 100, the evaluating unit 210a may generate the positional information corresponding to the converted value in the generating unit 210a3, and classify the individual into any one of the categories using the converted value in the classifying unit 210a4.

When the client apparatus 200 receives the value of the formula or the converted value and the positional information from the evaluating apparatus 100, the evaluating unit 210a may classify the individual into any one of the categories using the value of the formula or the converted value in the classifying unit 210a4.

### 2-3. Other Embodiments

In addition to the second embodiment described above, the evaluating apparatus, the calculating apparatus, the evaluating method, the calculating method, the evaluating program, the calculating program, the recording medium, the evaluating system, and the terminal apparatus according to the present invention can be practiced in various different embodiments within the technological scope of the claims.

Of the processings described in the second embodiment, all or a part of the processings described as automatically performed ones may be manually performed, or all or a part of the processings described as manually performed ones may be also automatically performed by known methods.

In addition, the processing procedures, the control procedures, the specific names, the information including parameters such as registered data of various processings and retrieval conditions, the screen examples, and the database configuration shown in the description and the drawings may be arbitrarily modified unless otherwise specified.

The components of the evaluating apparatus 100 shown in the figures are functionally conceptual and therefore not be physically configured as shown in the figures.

For example, for the operational functions provided in the evaluating apparatus 100, in particular, for the operational functions performed in the control device 102, all or part thereof may be implemented by the CPU (Central Processing Unit) and programs interpreted and executed in the CPU, or may be implemented by wired-logic hardware. The program is recorded in a non-transitory tangible computer-readable recording medium including programmed instructions for making an information processing apparatus execute the evaluating method or the calculating method according to the present invention, and is mechanically read as needed by the evaluating apparatus 100. More specifically, computer programs to give instructions to the CPU in cooperation with the OS (operating system) to perform various processes are recorded in the memory device 106 such as ROM or a HDD (hard disk drive). The computer programs are executed by being loaded to RAM, and form the control unit in cooperation with the CPU.

The computer programs may be stored in an application program server connected to the evaluating apparatus 100 via an arbitrary network, and all or part thereof can be downloaded as necessary.

The evaluating program or the calculating program according to the present invention may be stored in the non-transitory tangible computer-readable recording medium, or can be configured as a program product. The “recording medium” mentioned here includes any “portable physical medium” such as a memory card, a USB (universal serial bus) memory, an SD (secure digital) card, a flexible disk, a magneto-optical disc, ROM, EPROM (erasable programmable read only memory), EEPROM (registered trademark) (electronically erasable and programmable read only memory), CD-ROM (compact disk read only memory), MO (magneto-optical disk), DVD (digital versatile disk), and Blu-ray (registered trademark) Disc.

The “program” mentioned here is a data processing method described in an arbitrary language or description method, and therefore any form such as a source code and a binary code is acceptable. The “program” is not necessarily limited to a program configured as a single unit, and, therefore, includes those dispersively configured as a plurality of modules and libraries and those in which the function of the program is achieved in cooperation with separate programs represented as OS (operating system). Any known configuration and procedures can be used as a specific configuration and reading procedure to read a recording medium by each apparatus shown in the embodiments, an installation procedure after the reading, and the like.

The various databases and the like stored in the memory device 106 is a storage unit such as a memory device such as RAM and ROM, a fixed disk drive such as a hard disk, a flexible disk, or an optical disc. The memory device 106 stores therein various programs, tables, databases, files for Web (World Wide Web) pages, and the like used to perform various processes and to provide Web sites.

The evaluating apparatus 100 may be configured as an information processing apparatus such as known personal computer and work station, or may be configured as the information processing apparatus connected to an arbitrary peripheral device. The evaluating apparatus 100 may be provided by installing software (including the programs and the data, etc.) to cause the information processing apparatus to implement the evaluating method or the calculating method according to the present invention.

Furthermore, a specific configuration of dispersion or integration of the apparatuses is not limited to the shown one. The apparatuses can be configured by functionally or physically dispersing or integrating all or part of the apparatuses in arbitrary units according to various types of additions or the like or according to functional loads. In other words, the embodiments may be implemented in arbitrary combinations thereof or an embodiment may be selectively implemented.

### Example 1

An observational clinical study was conducted to analyze the relation between the measurement of amino acid and metabolite concentrations in the peripheral blood of patients using an immune checkpoint inhibitor and the therapeutic effects. Blood samples were obtained from patients with advanced or recurrent lung cancer scheduled to receive treatment with an immune checkpoint inhibitor, and analysis was conducted for 21 kinds of amino acids (Glu, Arg, Orn, Cit, His, Val, Phe, Tyr, Met, Pro, Trp, Asn, Leu, Lys, Thr, Ile, Gln, Ala, Ser, a-ABA, and Gly) and 15 kinds of amino acid-related metabolites (AnthA, hAnthA, hIAA, hKyn, hTrp, IAA, ILA, Kyn, KynA, NFKyn, NP, PA, QA, Serot, and XA). Nivolumab or pembrolizumab, which is an anti-PD-1 antibody, was used as an antibody drug.

Blood samples were taken from peripheral veins before the start of treatment and six weeks after the start of treatment. The blood samples were cooled immediately after being taken, and plasma was separated from the blood samples. The concentration values of free amino acids and amino acid-related metabolites in plasma were measured using an LC-MS analyzer or an LC-MS/MS analyzer according to the measurement method (A) explained in the foregoing embodiment. Patient background information, such as progression and histological classification of cancer, and clinical test values, was collected from the patients. The prognosis of treatment (overall survival) for each patient was followed up for up to two years from the start of treatment (the average follow-up period of 272 days).

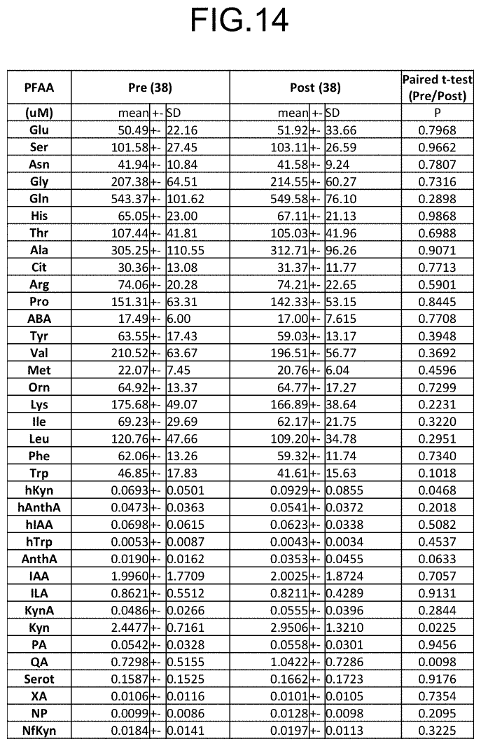

The changes in blood concentrations of the 21 kinds of amino acids and the 15 kinds of amino acid-related metabolites before and after the start of treatment are listed in FIG. 14.

FIG. 14 depicts the mean concentration value (pre) before the start of treatment and the mean concentration value (post) six weeks after the start of treatment for each amino acid and each amino acid-related metabolite. The results of the paired t-test showed that hKyn, Kyn, and QA significantly increased six weeks after the start of treatment compared with before the start of treatment (p<0.05).

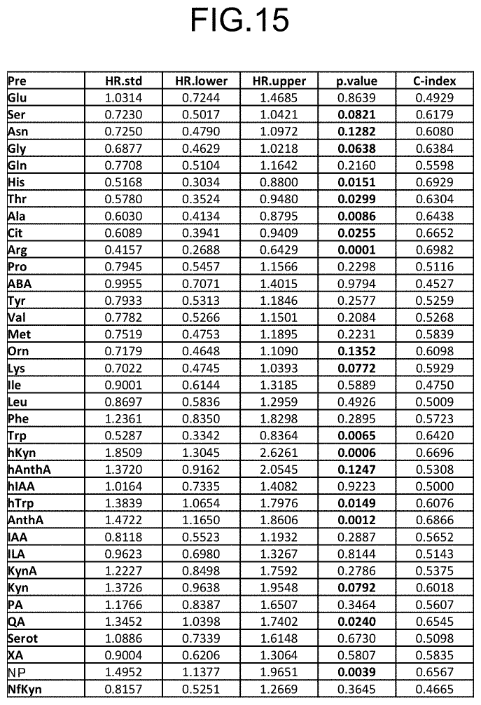

Correlation analysis between the concentration values of the 21 kinds of amino acids and the 15 kinds of amino acid-related metabolites before the start of treatment and the prognosis of treatment (overall survival) was performed. The analyzed 53 patients with lung cancer had non-small cell lung cancer. Nivolumab was used in 25 subjects and pembrolizumab was used in 28 subjects. Thirty five deaths were found. The analysis results are listed in FIG. 15. FIG. 15 depicts the hazard ratio, the P-value, and the C-Index in univariate COX hazard analysis between each amino acid and each amino acid-related metabolite and overall survival. Six kinds, namely His, Thr, Ala, Cit, Arg, and Trp were identified as amino acids with significant change, and five kinds, namely hKyn, hTrp, AnthA, QA, and NP were identified as amino acid-related metabolites with significant change. It was shown that the blood concentration values before the start of treatment of the six kinds of amino acids and the five kinds of amino acid-related metabolites showing a significant correlation with the prognosis of treatment could be indicators for predicting the prognosis of treatment with the immune checkpoint inhibitor.

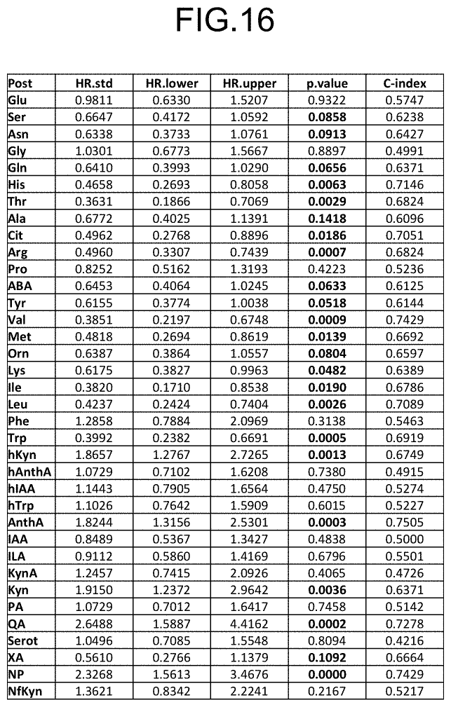

Correlation analysis between the concentration values of the 21 kinds of amino acids and the 15 kinds of amino acid-related metabolites six weeks after the start of treatment and the prognosis of treatment (overall survival) was performed. The analysis results are listed in FIG. 16. FIG. 16 depicts the hazard ratio, the P-value, and the C-Index in univariate COX hazard analysis between each amino acid and each amino acid-related metabolite and the prognosis of treatment (overall survival). Ten kinds, namely His, Thr, Cit, Arg, Val, Met, Lys, Ile, Leu, and Trp were identified as amino acids with significant change, and five kinds, namely hKyn, AnthA, Kyn, QA, and NP were identified as amino acid-related metabolites with significant change. It was shown that the blood concentration values six weeks after the start of treatment of the ten kinds of amino acids and the five kinds of amino acid-related metabolites showing a significant correlation with the prognosis of treatment could be indicators for predicting the prognosis of treatment with the immune checkpoint inhibitor.

Multivariate analysis of the influence of the concentration values of amino acids and amino acid-related metabolites in plasma on overall survival was performed using a Cox proportional hazards model, and discriminants for predicting the prognosis of treatment with the immune checkpoint inhibitor were prepared.

In selecting explanatory variables in multivariate analysis using the Cox proportional hazards model, when only amino acids were used as explanatory variables, the 21 kinds of amino acids were included regardless of the presence or absence of significant differences, and when combinations of amino acids and amino acid-related metabolites were used as explanatory variables, the explanatory variables were selected based on P<0.15 in univariate analysis. Discriminants with two explanatory variables, discriminants with three explanatory variables, discriminants with four explanatory variables, discriminants with five explanatory variables, and discriminants with six explanatory variables were created. Combinations of explanatory variables were listed as follows for discriminants with two explanatory variables ranked in top 100 discriminant performances, discriminants with three explanatory variables ranked in top 100 discriminant performances, discriminants with four explanatory variables ranked in top 100 discriminant performances, discriminants with five explanatory variables ranked in top 100 discriminant performances, and discriminants with six explanatory variables ranked in top 100 discriminant performances.

The combinations of explanatory variables in the discriminants with two explanatory variables ranked in top 100 discriminant performances that were obtained from the amino acids before the start of treatment are listed in the following [101. Formulae with two explanatory variables obtained from amino acids before the start of treatment]. The combinations of explanatory variables in the discriminants with three explanatory variables ranked in top 100 discriminant performances that were obtained from the amino acids before the start of treatment are listed in the following [102. Formulae with three explanatory variables obtained from amino acids before the start of treatment]. The combinations of explanatory variables in the discriminants with four explanatory variables ranked in top 100 discriminant performances that were obtained from the amino acids before the start of treatment are listed in the following [103. Formulae with four explanatory variables obtained from amino acids before the start of treatment]. The combinations of explanatory variables in the discriminants with five explanatory variables ranked in top 100 discriminant performances that were obtained from the amino acids before the start of treatment are listed in the following [104. Formulae with five explanatory variables obtained from amino acids before the start of treatment]. The combinations of explanatory variables in the discriminants with six explanatory variables ranked in top 100 discriminant performances that were obtained from the amino acids before the start of treatment are listed in the following [105. Formulae with six explanatory variables obtained from amino acids before the start of treatment].

The combinations of explanatory variables in the discriminants with two explanatory variables ranked in top 100 discriminant performances that were obtained from the amino acids and amino acid-related metabolites before the start of treatment are listed in the following [106. Formulae with two explanatory variables obtained from amino acids and amino acid-related metabolites before the start of treatment]. The combinations of explanatory variables in the discriminants with three explanatory variables ranked in top 100 discriminant performances that were obtained from the amino acids and amino acid-related metabolites before the start of treatment are listed in the following [107. Formulae with three explanatory variables obtained from amino acids and amino acid-related metabolites before the start of treatment]. The combinations of explanatory variables in the discriminants with four explanatory variables ranked in top 100 discriminant performances that were obtained from the amino acids and amino acid-related metabolites before the start of treatment are listed in the following [108. Formulae with four explanatory variables obtained from amino acids and amino acid-related metabolites before the start of treatment]. The combinations of explanatory variables in the discriminants with five explanatory variables ranked in top 100 discriminant performances that were obtained from the amino acids and amino acid-related metabolites before the start of treatment are listed in the following [109. Formulae with five explanatory variables obtained from amino acids and amino acid-related metabolites before the start of treatment]. The combinations of explanatory variables in the discriminants with six explanatory variables ranked in top 100 discriminant performances that were obtained from the amino acids and amino acid-related metabolites before the start of treatment are listed in the following [110. Formulae with six explanatory variables obtained from amino acids and amino acid-related metabolites before the start of treatment].

The combinations of explanatory variables in the discriminants with two explanatory variables ranked in top 100 discriminant performances that were obtained from the amino acids six weeks after the start of treatment are listed in the following [111. Formulae with two explanatory variables obtained from amino acids after the start of treatment]. The combinations of explanatory variables in the discriminants with three explanatory variables ranked in top 100 discriminant performances that were obtained from the amino acids six weeks after the start of treatment are listed in the following [112. Formulae with three explanatory variables obtained from amino acids after the start of treatment]. The combinations of explanatory variables in the discriminants with four explanatory variables ranked in top 100 discriminant performances that were obtained from the amino acids six weeks after the start of treatment are listed in the following [113. Formulae with four explanatory variables obtained from amino acids after the start of treatment]. The combinations of explanatory variables in the discriminants with five explanatory variables ranked in top 100 discriminant performances that were obtained from the amino acids six weeks after the start of treatment are listed in the following [114. Formulae with five explanatory variables obtained from amino acids after the start of treatment]. The combinations of explanatory variables in the discriminants with six explanatory variables ranked in top 100 discriminant performances that were obtained from the amino acids six weeks after the start of treatment are listed in the following [115. Formulae with six explanatory variables obtained from amino acids after the start of treatment].

The combinations of explanatory variables in the discriminants with two explanatory variables ranked in top 100 discriminant performances that were obtained from the amino acids and amino acid-related metabolites six weeks after the start of treatment are listed in the following [116. Formulae with two explanatory variables obtained from amino acids and amino acid-related metabolites after the start of treatment]. The combinations of explanatory variables in the discriminants with three explanatory variables ranked in top 100 discriminant performances that were obtained from the amino acids and amino acid-related metabolites six weeks after the start of treatment are listed in the following [117. Formulae with three explanatory variables obtained from amino acids and amino acid-related metabolites after the start of treatment]. The combinations of explanatory variables in the discriminants with four explanatory variables ranked in top 100 discriminant performances that were obtained from the amino acids and amino acid-related metabolites six weeks after the start of treatment are listed in the following [118. Formulae with four explanatory variables obtained from amino acids and amino acid-related metabolites after the start of treatment]. The combinations of explanatory variables in the discriminants with five explanatory variables ranked in top 100 discriminant performances that were obtained from the amino acids and amino acid-related metabolites six weeks after the start of treatment are listed in the following [119. Formulae with five explanatory variables obtained from amino acids and amino acid-related metabolites after the start of treatment]. The combinations of explanatory variables in the discriminants with six explanatory variables ranked in top 100 discriminant performances that were obtained from the amino acids and amino acid-related metabolites six weeks after the start of treatment are listed in the following [120. Formulae with six explanatory variables obtained from amino acids and amino acid-related metabolites after the start of treatment].

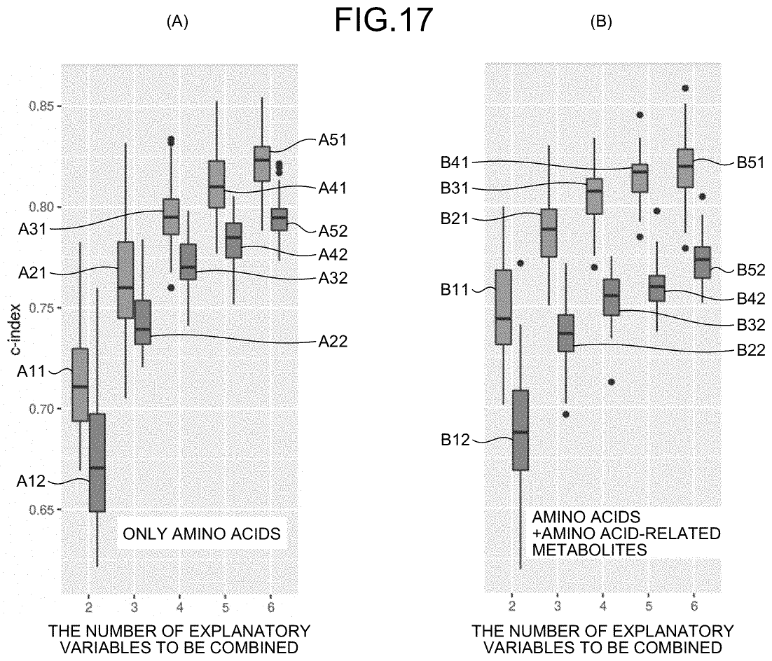

FIG. 17 depicts the distribution of the C-index representing the performance of the discriminants listed in [101. Formulae with two explanatory variables obtained from amino acids before the start of treatment] to [120. Formulae with six explanatory variables obtained from amino acids and amino acid-related metabolites after the start of treatment]. The horizontal axis is the number of explanatory variables to be combined, and the vertical axis is the C-index. In the figure, the distributions shown in (A) are the distributions of the C-index corresponding to the discriminants obtained from the concentration values of amino acids, and the distributions shown in (B) are the distributions of the C-index corresponding to the discriminants obtained from the concentration values of amino acids and amino acid-related metabolites. The distributions corresponding to symbols A11, A21, A31, A41, A51, B11, B21, B31, B41, and B51 are the distributions of the C-index corresponding to the discriminants obtained from the concentration values before the start of treatment, and the distributions corresponding to symbols A12, A22, A32, A42, A52, B12, B22, B32, B42, and B52 are the distributions of the C-index corresponding to the discriminants obtained from the concentration values six weeks after the start of treatment.

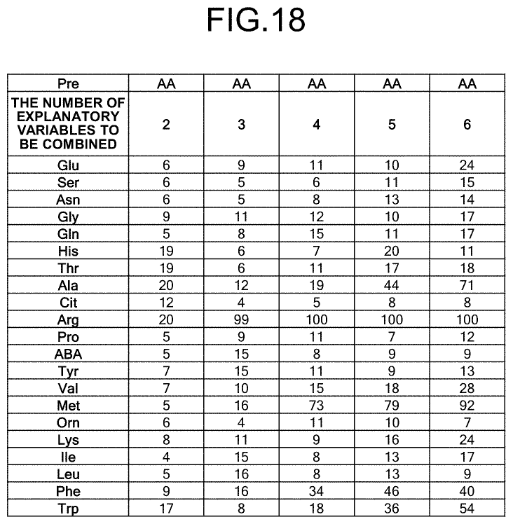

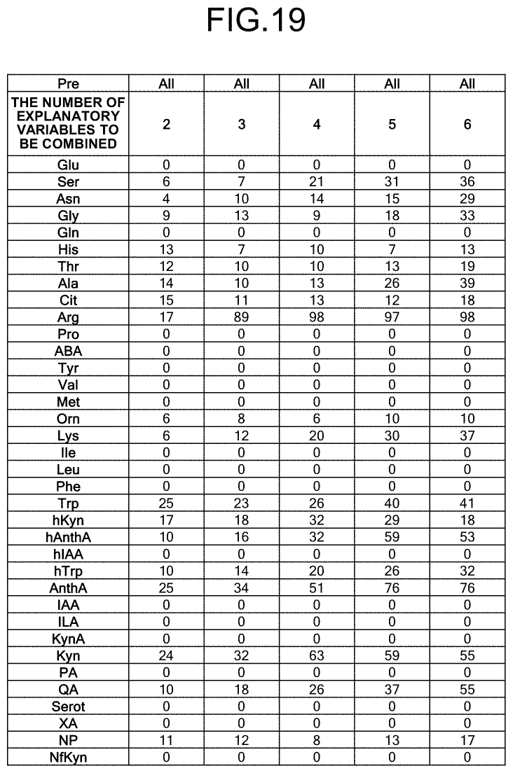

FIG. 18 depicts the frequency of occurrence of explanatory variables in the discriminants listed in [101. Formulae with two explanatory variables obtained from amino acids before the start of treatment] to [105. Formulae with six explanatory variables obtained from amino acids before the start of treatment]. FIG. 19 depicts the frequency of occurrence of explanatory variables in the discriminants listed in [106. Formulae with two explanatory variables obtained from amino acids and amino acid-related metabolites before the start of treatment] to [110. Formulae with six explanatory variables obtained from amino acids and amino acid-related metabolites before the start of treatment].

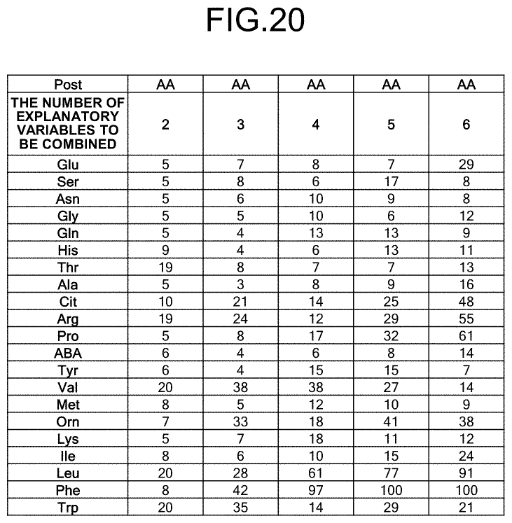

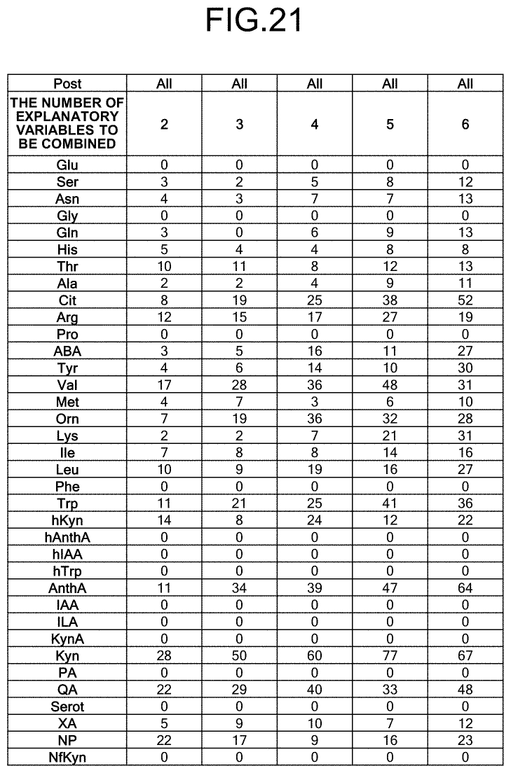

FIG. 20 depicts the frequency of occurrence of explanatory variables in the discriminants listed in [111. Formulae with two explanatory variables obtained from amino acids after the start of treatment] to [115. Formulae with six explanatory variables obtained from amino acids after the start of treatment]. FIG. 21 depicts the frequency of occurrence of explanatory variables in the discriminants listed in [116. Formulae with two explanatory variables obtained from amino acids and amino acid-related metabolites after the start of treatment] to [120. Formulae with six explanatory variables obtained from amino acids and amino acid-related metabolites after the start of treatment].

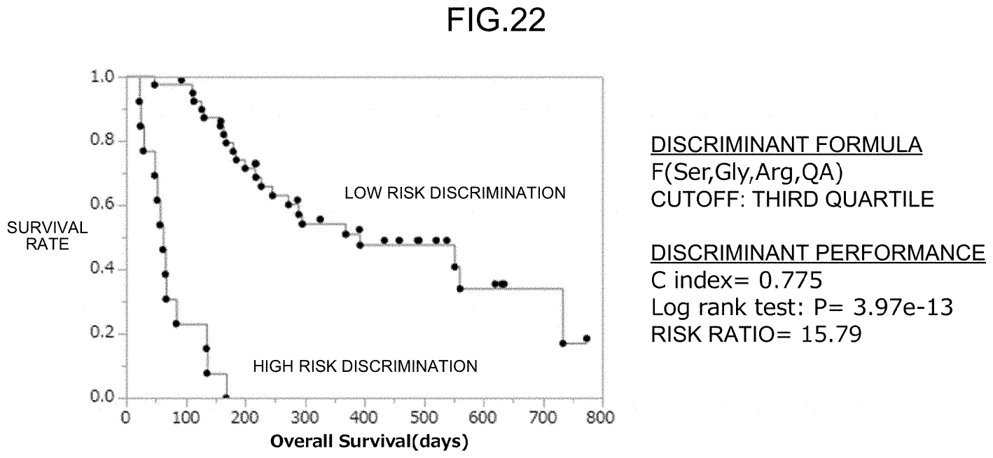

FIG. 22 is a graph for explaining the discriminant performance of a discriminant prepared to minimize Akaike's information criterion (AIC) by the stepwise method, among the discriminants. The discriminant was prepared with four parameters including Ser, Gly, Arg, and QA. The C-index representing the performance of the prepared discriminant was 0.775, and the P-value of the log-rank test was 3.97×10⁻¹³ and the risk ratio was 15.79 when the upper quartile was used as the cutoff value. The prepared discriminant enabled highly accurate prediction of the prognosis of treatment with immune checkpoint inhibitor therapy.

### Example 2

For the patients with lung cancer described in Example 1, the development of adverse effects associated with treatment with the immune checkpoint inhibitor was studied. The adverse effects associated with treatment with the immune checkpoint inhibitor were identified in 22 subjects in total, and a total of 43 cases were identified, namely interstitial pneumonia (7 cases), pneumonia (10 cases), rash/itch (10 cases), intestinal inflammation/diarrhea (6 cases), fever (3 cases), hyperthyroidism (2 cases), fatigue (2 cases), arthralgia (1 case), dysgeusia (1 case), and AST and ALT abnormalities (1 case). Of these, a total of 5 cases, namely interstitial pneumonia (2 cases), pneumonia (2 cases), and enteritis (1 case) were identified as the adverse effects with Grade 3 or higher in common terminology criteria for adverse events (CTCAE).

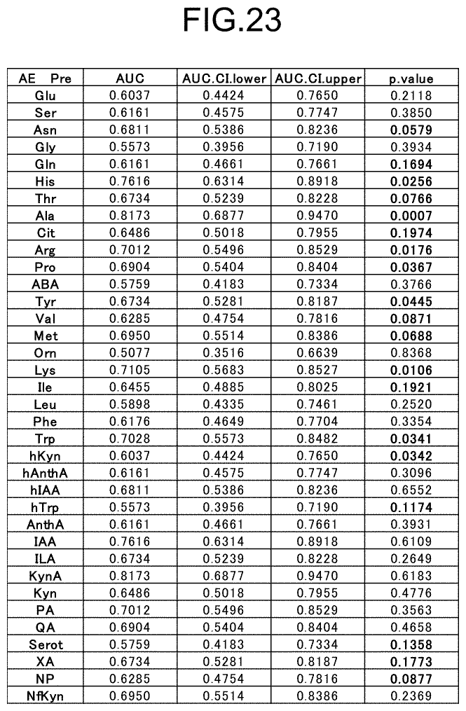

Correlation analysis between the concentration values of the 21 kinds of amino acids and the 15 kinds of amino acid-related metabolites before the start of treatment and the risk of developing an adverse effect associated with the treatment with the immune checkpoint inhibitor was performed. The analysis results are listed in FIG. 23. FIG. 23 depicts the ROC_AUC and the P-value in the univariate correlation analysis between each amino acid and the risk of developing an adverse effect. Seven kinds, namely His, Ala, Arg, Pro, Tyr, Lys, and Trp were identified as amino acids with significant change, and one kind, namely hKyn was identified as an amino acid-related metabolite with significant change. It was shown that the seven kinds of amino acids and the one kind of amino acid-related metabolite showing a significant correlation with the risk of developing an adverse effect could be indicators for predicting the risk of developing an adverse effect with the immune checkpoint inhibitor.

Multivariate analysis of the influence of the concentration values of amino acids and amino acid-related metabolites in plasma on the risk of developing an adverse effect was performed using a logistic regression model, and discriminants for discriminating the risk of developing an adverse effect with the immune checkpoint inhibitor were prepared.

In selecting explanatory variables in multivariate analysis using the logistic regression model, when only amino acids were used as explanatory variables, the 21 kinds were included regardless of the presence or absence of significant differences, and when combinations of amino acids and amino acid-related metabolites were used as explanatory variables, the explanatory variables were selected based on P<0.20 in univariate analysis. Discriminants with two explanatory variables, discriminants with three explanatory variables, discriminants with four explanatory variables, discriminants with five explanatory variables, and discriminants with six explanatory variables were created. Combinations of explanatory variables were listed as follows for discriminants with two explanatory variables ranked in top 100 discriminant performances, discriminants with three explanatory variables ranked in top 100 discriminant performances, discriminants with four explanatory variables ranked in top 100 discriminant performances, discriminants with five explanatory variables ranked in top 100 discriminant performances, and discriminants with six explanatory variables ranked in top 100 discriminant performances.

The combinations of explanatory variables in the discriminants with two explanatory variables ranked in top 100 discriminant performances that were obtained from the amino acids before the start of treatment are listed in the following [201. Formulae with two explanatory variables obtained from amino acids before the start of treatment]. The combinations of explanatory variables in the discriminants with three explanatory variables ranked in top 100 discriminant performances that were obtained from the amino acids before the start of treatment are listed in the following [202. Formulae with three explanatory variables obtained from amino acids before the start of treatment]. The combinations of explanatory variables in the discriminants with four explanatory variables ranked in top 100 discriminant performances that were obtained from the amino acids before the start of treatment are listed in the following [203. Formulae with four explanatory variables obtained from amino acids before the start of treatment]. The combinations of explanatory variables in the discriminants with five explanatory variables ranked in top 100 discriminant performances that were obtained from the amino acids before the start of treatment are listed in the following [204. Formulae with five explanatory variables obtained from amino acids before the start of treatment]. The combinations of explanatory variables in the discriminants with six explanatory variables ranked in top 100 discriminant performances that were obtained from the amino acids before the start of treatment are listed in the following [205. Formulae with six explanatory variables obtained from amino acids before the start of treatment].

The combinations of explanatory variables in the discriminants with two explanatory variables ranked in top 100 discriminant performances that were obtained from the amino acids and amino acid-related metabolites before the start of treatment are listed in the following [206. Formulae with two explanatory variables obtained from amino acids and amino acid-related metabolites before the start of treatment]. The combinations of explanatory variables in the discriminants with three explanatory variables ranked in top 100 discriminant performances that were obtained from the amino acids and amino acid-related metabolites before the start of treatment are listed in the following [207. Formulae with three explanatory variables obtained from amino acids and amino acid-related metabolites before the start of treatment]. The combinations of explanatory variables in the discriminants with four explanatory variables ranked in top 100 discriminant performances that were obtained from the amino acids and amino acid-related metabolites before the start of treatment are listed in the following [208. Formulae with four explanatory variables obtained from amino acids and amino acid-related metabolites before the start of treatment]. The combinations of explanatory variables in the discriminants with five explanatory variables ranked in top 100 discriminant performances that were obtained from the amino acids and amino acid-related metabolites before the start of treatment are listed in the following [209. Formulae with five explanatory variables obtained from amino acids and amino acid-related metabolites before the start of treatment]. The combinations of explanatory variables in the discriminants with six explanatory variables ranked in top 100 discriminant performances that were obtained from the amino acids and amino acid-related metabolites before the start of treatment are listed in the following [210. Formulae with six explanatory variables obtained from amino acids and amino acid-related metabolites before the start of treatment].

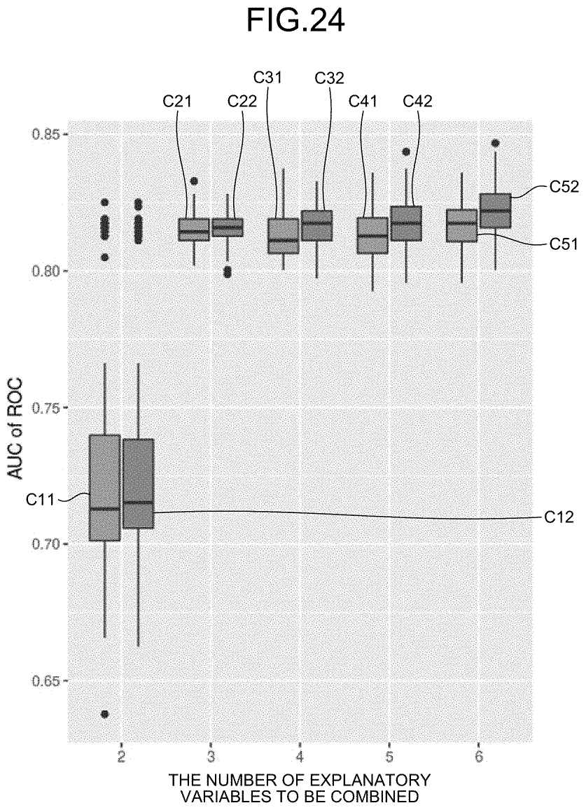

FIG. 24 depicts the distribution of the ROC_AUC representing the performance of the discriminants listed in [201. Formulae with two explanatory variables obtained from amino acids before the start of treatment] to [210. Formulae with six explanatory variables obtained from amino acids and amino acid-related metabolites before the start of treatment]. The horizontal axis is the number of explanatory variables to be combined, and the vertical axis is the ROC_AUC. In the figure, the distributions corresponding to symbols C11, C21, C31, C41, and C51 are the distributions of ROC_AUC corresponding to the discriminants obtained from the concentration values of amino acids, and the distributions corresponding to symbols C12, C22, C32, C42, and C52 are the distributions of ROC_AUC corresponding to the discriminants obtained from the concentration values of amino acids and amino acid-related metabolites. The discriminants prepared in this example demonstrated that a highly accurate discriminant for predicting an adverse effect developed after the start of treatment was able to be prepared.

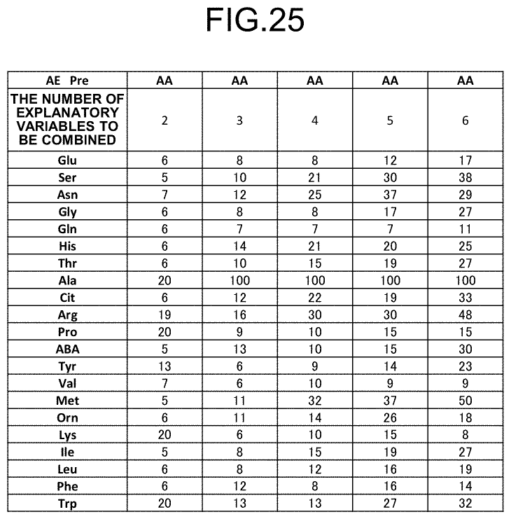

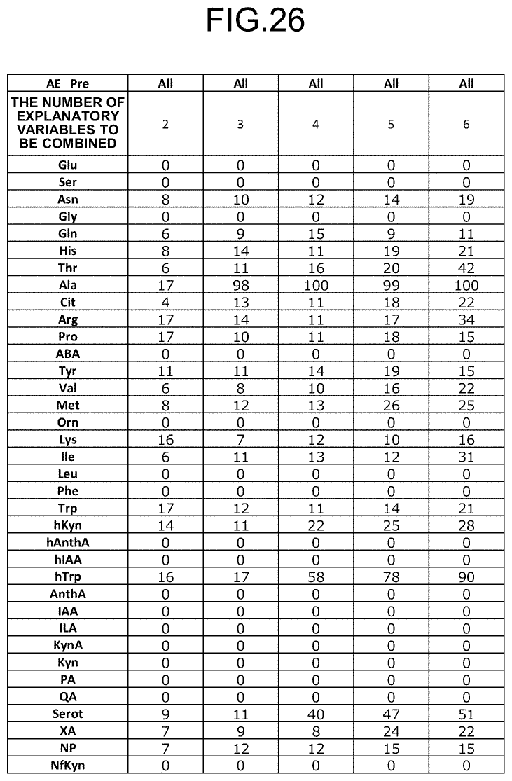

FIG. 25 depicts the frequency of occurrence of explanatory variables in the discriminants listed in [201. Formulae with two explanatory variables obtained from amino acids before the start of treatment] to [205. Formulae with six explanatory variables obtained from amino acids before the start of treatment]. FIG. 26 depicts the frequency of occurrence of explanatory variables in the discriminants listed in [206. Formulae with two explanatory variables obtained from amino acids and amino acid-related metabolites before the start of treatment] to [210. Formulae with six explanatory variables obtained from amino acids and amino acid-related metabolites before the start of treatment].

### Example 3

For the patients with lung cancer described in Example 1, analysis was also conducted for progression-free survival (PFS) and the response rate that are indicators of the prognosis of treatment with the immune checkpoint inhibitor. Blood samples were obtained from patients with advanced or recurrent lung cancer scheduled to receive treatment with an immune checkpoint inhibitor, and analysis was conducted for 19 kinds of amino acids (Arg, Orn, Cit, His, Val, Phe, Tyr, Met, Pro, Trp, Asn, Leu, Lys, Thr, Ile, Gln, Ala, Ser, and Gly) and 8 kinds of amino acid-related metabolites (AnthA, hKyn, hTrp, Kyn, KynA, NP, QA, and XA). Nivolumab or pembrolizumab, which is an anti-PD-1 antibody, was used as an antibody drug.

Blood samples were taken from peripheral veins before the start of treatment. The blood samples were cooled immediately after being taken, and plasma was separated from the blood samples. The concentration values of free amino acids and amino acid-related metabolites in plasma were measured using an LC-MS analyzer or an LC-MS/MS analyzer according to the measurement method (A) explained in the foregoing embodiment. Patient background information, such as progression and histological classification of cancer, and clinical test values, was collected from the patients. The response rate to treatment and the prognosis of treatment (progression-free survival (PFS)) for each patient were followed up for up to two years from the start of treatment (the average follow-up period 272 days).

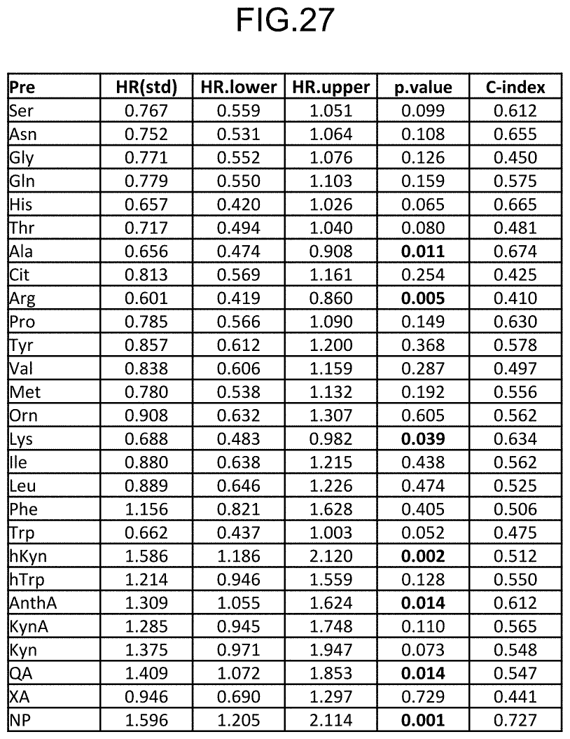

Correlation analysis between the concentration values of the 19 kinds of amino acids and the 8 kinds of amino acid-related metabolites before the start of treatment and the prognosis of treatment (progression-free survival (PFS)) was performed. In the analysis of PFS, event occurrence was identified in 46 cases out of the analyzed 53 patients with lung cancer. The analysis results are listed in FIG. 27. FIG. 27 depicts the hazard ratio, the P-value, and the C-Index in univariate COX hazard analysis between each amino acid and each amino acid-related metabolite and PFS. Three kinds, namely Ala, Arg, and Lys were identified as amino acids with significant change, and four kinds, namely hKyn, AnthA, QA, and NP were identified as amino acid-related metabolites with significant (P<0.05) change (refer to the P-values indicated by boldface in FIG. 27). It was shown that the blood concentration values before the start of treatment of the three kinds of amino acids and the four kinds of amino acid-related metabolites showing a significant correlation with the prognosis of treatment could be indicators for predicting the prognosis of treatment with the immune checkpoint inhibitor.

Multivariate analysis of the influence of the concentration values of amino acids and amino acid-related metabolites in plasma on PFS was performed using a Cox proportional hazards model, and linear discriminants for predicting the prognosis of treatment with the immune checkpoint inhibitor were prepared. Deaths during the follow-up period were treated as events, and patients who stopped treatment due to adverse effects and who failed to be followed up due to changing hospitals or the like were treated as censoring.

In selecting explanatory variables in multivariate analysis using the Cox proportional hazards model, when only amino acids were used as explanatory variables, the 19 kinds of amino acids were included regardless of the presence or absence of significant differences, and when combinations of amino acids and amino acid-related metabolites were used as explanatory variables, the explanatory variables were selected based on P<0.20 in univariate analysis. Discriminants with two explanatory variables, discriminants with three explanatory variables, discriminants with four explanatory variables, discriminants with five explanatory variables, and discriminants with six explanatory variables were created.

The combinations of explanatory variables in the discriminants with two explanatory variables ranked in top 99 discriminant performances and the C-index are listed in the following [301. Formulae with two explanatory variables obtained from amino acids before the start of treatment with PFS as evaluation index]. The combinations of explanatory variables in the discriminants with three explanatory variables ranked in top 100 discriminant performances and the C-index are listed in the following [302. Formulae with three explanatory variables obtained from amino acids before the start of treatment with PFS as evaluation index]. The combinations of explanatory variables in the discriminants with four explanatory variables ranked in top 100 discriminant performances and the C-index are listed in the following [303. Formulae with four explanatory variables obtained from amino acids before the start of treatment with PFS as evaluation index]. The combinations of explanatory variables in the discriminants with five explanatory variables ranked in top 100 discriminant performances and the C-index are listed in the following [304. Formulae with five explanatory variables obtained from amino acids before the start of treatment with PFS as evaluation index]. The combinations of explanatory variables in the discriminants with six explanatory variables ranked in top 100 discriminant performances and the C-index are listed in the following [305. Formulae with six explanatory variables obtained from amino acids before the start of treatment with PFS as evaluation index]. These combinations were obtained from the amino acids before the start of treatment with PFS as the evaluation index.

The combinations of explanatory variables in the discriminants with two explanatory variables ranked in top 100 discriminant performances and the C-index are listed in the following [306. Formulae with two explanatory variables obtained from amino acids and amino acid-related metabolites before the start of treatment with PFS as evaluation index]. The combinations of explanatory variables in the discriminants with three explanatory variables ranked in top 100 discriminant performances and the C-index are listed in the following [307. Formulae with three explanatory variables obtained from amino acids and amino acid-related metabolites before the start of treatment with PFS as evaluation index]. The combinations of explanatory variables in the discriminants with four explanatory variables ranked in top 100 discriminant performances and the C-index are listed in the following [308. Formulae with four explanatory variables obtained from amino acids and amino acid-related metabolites before the start of treatment with PFS as evaluation index]. The combinations of explanatory variables in the discriminants with five explanatory variables ranked in top 100 discriminant performances and the C-index are listed in the following [309. Formulae with five explanatory variables obtained from amino acids and amino acid-related metabolites before the start of treatment with PFS as evaluation index]. The combinations of explanatory variables in the discriminants with six explanatory variables ranked in top 100 discriminant performances and the C-index are listed in the following [310. Formulae with six explanatory variables obtained from amino acids and amino acid-related metabolites before the start of treatment with PFS as evaluation index]. These combinations were obtained from the amino acids and amino acid-related metabolites before the start of treatment with PFS as the evaluation index.

The response rate to treatment with the immune checkpoint inhibitor was analyzed. In order to determine the therapeutic effect of the immune checkpoint inhibitor, the best response evaluation was performed according to the RECIST guidelines Version 1.1 (EUROPEAN JOURNAL OF CANCER 45 (2009) 228-247). There were no case of complete response (CR), but there were 16 cases of partial response (PR), 9 cases of stable disease (SD), and 28 cases of progressive disease (PD). When the PR evaluation was considered as “respond”, the response rate was 30.2%.

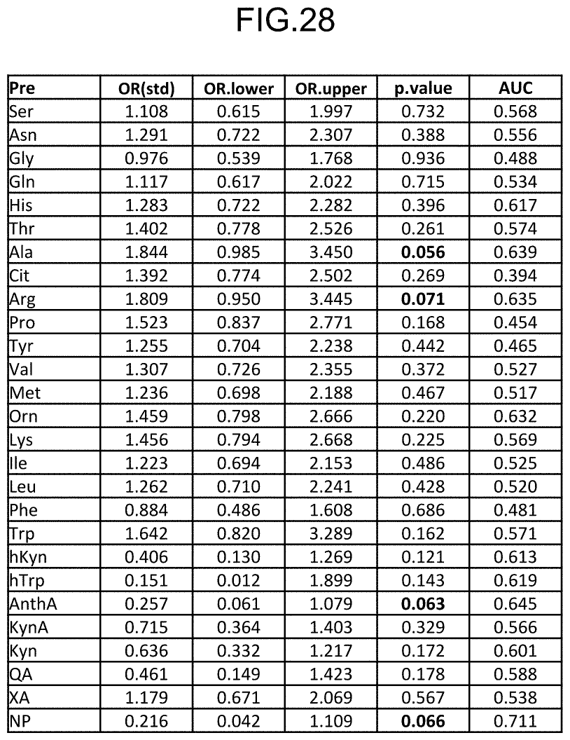

Correlation analysis between the concentration values of the 19 kinds of amino acids and the 8 kinds of amino acid-related metabolites before the start of treatment and the response rate to treatment was performed. The analysis results are listed in FIG. 28. FIG. 28 depicts the odds ratio, the P-value, and the ROC_AUC in univariate correlation analysis between each amino acid and each amino acid-related metabolite and the response rate to treatment. No explanatory variables with significant differences were identified, but two kinds, namely Ala and Arg were identified as amino acids with marginal significance (P<0.1), and two kinds, namely AnthA and NP were identified as amino acid-related metabolites with marginal significance (refer to the P-values indicated by boldface in FIG. 28).

Multivariate analysis of the influence of the concentration values of amino acids and amino acid-related metabolites in plasma on the response rate to treatment was performed using a logistic regression model, and discriminants for predicting the response rate to treatment with the immune checkpoint inhibitor were prepared.

In selecting explanatory variables in multivariate analysis using the logistic regression model, when only amino acids were used as explanatory variables, the 19 kinds of amino acids were included regardless of the presence or absence of significant differences, and when combinations of amino acids and amino acid-related metabolites were used as explanatory variables, the explanatory variables were selected based on P<0.30 in univariate analysis. Discriminants with two explanatory variables, discriminants with three explanatory variables, discriminants with four explanatory variables, discriminants with five explanatory variables, and discriminants with six explanatory variables were created.

The combinations of explanatory variables in the discriminants with two explanatory variables ranked in top 87 discriminant performances and the ROC_AUC are listed in the following [311. Formulae with two explanatory variables obtained from amino acids before the start of treatment with the response rate as evaluation index]. The combinations of explanatory variables in the discriminants with three explanatory variables ranked in top 98 discriminant performances and the ROC_AUC are listed in the following [312. Formulae with three explanatory variables obtained from amino acids before the start of treatment with the response rate as evaluation index]. The combinations of explanatory variables in the discriminants with four explanatory variables ranked in top 100 discriminant performances and the ROC_AUC are listed in the following [313. Formulae with four explanatory variables obtained from amino acids before the start of treatment with the response rate as evaluation index]. The combinations of explanatory variables in the discriminants with five explanatory variables ranked in top 100 discriminant performances and the ROC_AUC are listed in the following [314. Formulae with five explanatory variables obtained from amino acids before the start of treatment with the response rate as evaluation index]. The combinations of explanatory variables in the discriminants with six explanatory variables ranked in top 100 discriminant performances and the ROC_AUC are listed in the following [315. Formulae with six explanatory variables obtained from amino acids before the start of treatment with the response rate as evaluation index]. These combinations were obtained from the amino acids before the start of treatment with the response rate as the evaluation index.

The combinations of explanatory variables in the discriminants with two explanatory variables ranked in top 99 discriminant performances and the ROC_AUC are listed in the following [316. Formulae with two explanatory variables obtained from amino acids and amino acid-related metabolites before the start of treatment with the response rate as evaluation index]. The combinations of explanatory variables in the discriminants with three explanatory variables ranked in top 100 discriminant performances and the ROC_AUC are listed in the following [317. Formulae with three explanatory variables obtained from amino acids and amino acid-related metabolites before the start of treatment with the response rate as evaluation index]. The combinations of explanatory variables in the discriminants with four explanatory variables ranked in top 100 discriminant performances and the ROC_AUC are listed in the following [318. Formulae with four explanatory variables obtained from amino acids and amino acid-related metabolites before the start of treatment with the response rate as evaluation index]. The combinations of explanatory variables in the discriminants with five explanatory variables ranked in top 100 discriminant performances and the ROC_AUC are listed in the following [319. Formulae with five explanatory variables obtained from amino acids and amino acid-related metabolites before the start of treatment with the response rate as evaluation index]. The combinations of explanatory variables in the discriminants with six explanatory variables ranked in top 100 discriminant performances and the ROC_AUC are listed in the following [320. Formulae with six explanatory variables obtained from amino acids and amino acid-related metabolites before the start of treatment with the response rate as evaluation index]. These combinations were obtained from the amino acids and amino acid-related metabolites before the start of treatment with the response rate as the evaluation index.

[101. Formulae with two explanatory variables obtained from amino acids before the start of treatment]

Arg,Met, 0.76; Arg,Phe, 0.753; Arg,Ile, 0.741; Arg,Lys, 0.737; Arg,Tyr, 0.73; Arg,Leu, 0.729; His,Phe, 0.729; Gly,Arg, 0.729; Arg,ABA, 0.727; Glu,Arg, 0.725; His,Met, 0.725; Arg,Val, 0.724; Thr,Arg, 0.72; Arg,Pro, 0.718; Gln,Arg, 0.711; His,ABA, 0.711; Asn,Arg, 0.706; His,Leu, 0.706; Arg,Trp, 0.703; Ser,Arg, 0.702; Phe,Trp, 0.701; Gly,His, 0.701; His,Arg, 0.699; His,Val, 0.698; Ala,Arg, 0.697; Cit,Arg, 0.697; Arg,Orn, 0.696; His,Tyr, 0.696; Gly,Cit, 0.695; His,Pro, 0.694; Asn,His, 0.693; Gly,Trp, 0.691; His,Orn, 0.691; Ser,His, 0.69; Ala,Phe, 0.689; Glu,His, 0.689; Gln,His, 0.688; Gly,Ala, 0.687; His,Cit, 0.687; Asn,Cit, 0.687; Ala,Cit, 0.686; Met,Trp, 0.683; His,Lys, 0.683; Glu,Cit, 0.68; His,Thr, 0.679; Tyr,Trp, 0.679; Ser,Cit, 0.678; Cit,Phe, 0.676; His,Ala, 0.674; His,Trp, 0.673; Ala,Met, 0.668; Ala,Orn, 0.667; Ala,Leu, 0.666; Ala,Ile, 0.665; Leu,Trp, 0.664; ABA,Trp, 0.662; Ser,Ala, 0.661; Thr,Cit, 0.661; Ala,Tyr, 0.661; Cit,Tyr, 0.661; Val,Phe, 0.66; Glu,Trp, 0.66; Glu,Ala, 0.658; Gly,Orn, 0.658; Ile,Trp, 0.657; Ser,Trp, 0.657; Ala,Pro, 0.655; Ala,Val, 0.654; Gly,Lys, 0.654; Ala,ABA, 0.654; Cit,Val, 0.654; Ala,Trp, 0.653; Gln,Ala, 0.65; Thr,Phe, 0.649; Gly,Thr, 0.649; Orn,Trp, 0.648; Thr,Ala, 0.647; Ala,Lys, 0.646; Gly,Val, 0.645; Cit,Lys, 0.645; Asn,Ala, 0.645; Lys,Phe, 0.643; Pro,Trp, 0.642; Lys,Trp, 0.641; Gln,Trp, 0.638; Thr,ABA, 0.638; Tyr,Phe, 0.637; Asn,Trp, 0.636; Thr,Ile, 0.636; Thr,Tyr, 0.633; Glu,Thr, 0.631; Thr,Orn, 0.63; Ser,Thr, 0.629; Thr,Val, 0.629; Thr,Pro, 0.629; Thr,Lys, 0.627; Asn,Thr, 0.626; Gln,Thr, 0.625; Thr,Met, 0.622; Thr,Leu, 0.621

[102. Formulae with three explanatory variables obtained from amino acids before the start of treatment]

Ala,Arg,Met, 0.784; His,Arg,Met, 0.781; Arg,Met,Trp, 0.777; Ala,Arg,Phe, 0.776; Arg,Met,Phe, 0.771; Glu,Arg,Met, 0.771; Arg,Val,Phe, 0.769; Arg,Val,Met, 0.768; Arg,ABA,Met, 0.764; Arg,Tyr,Met, 0.763; Gln,Arg,Met, 0.763; Arg,Met,Leu, 0.763; Thr,Arg,Met, 0.762; Arg,Met,Lys, 0.762; Ser,Arg,Phe, 0.762; Arg,Met,Orn, 0.76; Ala,Arg,Lys, 0.76; Arg,Phe,Trp, 0.759; Arg,Met,Ile, 0.759; Cit,Arg,Met, 0.759; Arg,Pro,Met, 0.756; Asn,Arg,Phe, 0.755; Arg,Orn,Phe, 0.755; Arg,Ile,Phe, 0.754; Arg,Pro,Phe, 0.754; Arg,Leu,Phe, 0.754; Gly,Arg,Phe, 0.754; Arg,Tyr,Phe, 0.753; Arg,Lys,Phe, 0.752; His,Arg,Phe, 0.752; Cit,Arg,Phe, 0.75; Glu,Arg,Ile, 0.748; Gly,Thr,Arg, 0.748; Arg,Pro,Ile, 0.746; Arg,Tyr,Lys, 0.745; Arg,Pro,Lys, 0.745; Arg,Val,Ile, 0.743; Arg,Tyr,Ile, 0.742; Gly,Arg,Ile, 0.742; Thr,Arg,Ile, 0.742; Cit,Arg,Ile, 0.742; Arg,Lys,Leu, 0.741; Arg,Orn,Leu, 0.741; His,Arg,Ile, 0.74; Asn,Arg,Ile, 0.74; Arg,ABA,Tyr, 0.74; Gly,Arg,Lys, 0.74; Arg,Ile,Trp, 0.739; Ala,Arg,Ile, 0.739; Gln,Arg,Ile, 0.739; Gly,Gln,Arg, 0.739; Arg,Leu,Trp, 0.738; Ala,Arg,Tyr, 0.738; His,Arg,Lys, 0.738; Gly,Arg,ABA, 0.738; Arg,Pro,ABA, 0.737; Ser,Arg,Leu, 0.737; Gly,Arg,Leu, 0.737; Glu,Arg,ABA, 0.737; Arg,ABA,Ile, 0.736; Arg,Pro,Leu, 0.736; Glu,Arg,Leu, 0.736; Gln,Arg,ABA, 0.736; Gln,Arg,Leu, 0.735; Ala,Arg,Leu, 0.735; Arg,Pro,Val, 0.735; His,Arg,Leu, 0.734; Arg,Val,Lys, 0.734; Glu,Arg,Tyr, 0.734; Arg,Pro,Tyr, 0.734; Gln,Arg,Lys, 0.734; Arg,Tyr,Leu, 0.733; Arg,Val,Leu, 0.733; Asn,Arg,Tyr, 0.733; Thr,Arg,ABA, 0.732; Gln,Arg,Val, 0.731; Glu,Ala,Arg, 0.731; Ala,Orn,Phe, 0.73; Thr,Arg,Leu, 0.73; Arg,Tyr,Val, 0.73; Ser,Arg,Tyr, 0.73; Glu,Gly,Arg, 0.73; Arg,Val,Trp, 0.729; Gln,Arg,Tyr, 0.729; Arg,ABA,Leu, 0.729; Ser,Gly,Arg, 0.729; Thr,Arg,Tyr, 0.729; Arg,ABA,Trp, 0.728; Arg,ABA,Lys, 0.728; Gly,Arg,Trp, 0.728; Cit,Arg,Tyr, 0.728; Ser,Arg,ABA, 0.728; Glu,Asn,Arg, 0.727; His,Arg,ABA, 0.727; Ala,Arg,Val, 0.726; Ala,Arg,ABA, 0.726; Glu,Arg,Trp, 0.725; Gly,Ala,Arg, 0.723; Asn,Arg,ABA, 0.722; Ala,Arg,Pro, 0.721

[103. Formulae with four explanatory variables obtained from amino acids before the start of treatment]

Glu,Arg,Met,Trp, 0.798; Gly,Arg,Met,Trp, 0.796; Gly,Ala,Arg,Met, 0.793; Ala,Arg,Met,Lys, 0.793; Glu,Ala,Arg,Met, 0.792; Ala,Arg,Met,Trp, 0.79; Ala,Arg,Met,Leu, 0.789; His,Arg,Tyr,Met, 0.789; Gly,His,Arg,Met, 0.788; His,Arg,Met,Orn, 0.788; Arg,Met,Phe,Trp, 0.787; Ala,Arg,Met,Phe, 0.787; Thr,Ala,Arg,Met, 0.787; Arg,Met,Ile,Trp, 0.786; Gln,His,Arg,Met, 0.786; Ala,Arg,Val,Met, 0.785; Ala,Arg,Tyr,Met, 0.785; Ala,Cit,Arg,Met, 0.784; Ala,Arg,Tyr,Phe, 0.784; Ala,Arg,ABA,Met, 0.783; Gln,Ala,Arg,Met, 0.783; Ala,Arg,Pro,Met, 0.783; Arg,Val,Met,Trp, 0.782; Arg,Met,Lys,Trp, 0.782; His,Arg,Pro,Met, 0.782; Arg,Met,Leu,Phe, 0.781; His,Arg,Met,Leu, 0.781; Gly,Arg,Val,Met, 0.781; Gly,Arg,Met,Leu, 0.779; Gln,Arg,Met,Phe, 0.779; Asn,Arg,Met,Leu, 0.779; Glu,Arg,Val,Met, 0.778; Arg,Tyr,Met,Trp, 0.777; Gln,His,Arg,Phe, 0.777; Thr,Arg,Met,Trp, 0.776; Glu,Arg,Tyr,Met, 0.776; Gly,Arg,Met,Lys, 0.776; Glu,Arg,Met,Phe, 0.775; Ala,Arg,ABA,Phe, 0.775; Arg,ABA,Met,Phe, 0.774; Asn,Arg,Met,Ile, 0.773; Gly,Ala,Arg,Phe, 0.773; Asn,Gln,Arg,Met, 0.772; Glu,Asn,Arg,Met, 0.772; Gly,Arg,Tyr,Met, 0.772; Gln,Thr,Arg,Phe, 0.772; Asn,Arg,Val,Met, 0.771; Arg,Met,Ile,Phe, 0.771; Gln,Arg,Val,Phe, 0.771; Gly,Arg,ABA,Met, 0.771; Gly,Thr,Arg,Met, 0.77; Asn,Arg,Met,Orn, 0.77; Glu,Ala,Arg,Phe, 0.77; Arg,Val,Met,Orn, 0.769; Ser,Arg,Pro,Phe, 0.769; Gly,Arg,Val,Phe, 0.769; Asn,Arg,Pro,Met, 0.768; Ala,Arg,Orn,Phe, 0.768; Glu,Gln,Arg,Met, 0.768; Glu,Arg,Met,Orn, 0.768; Thr,Arg,Val,Phe, 0.768; Ser,Arg,Phe,Trp, 0.767; Arg,Pro,Met,Phe, 0.767; Arg,Val,Leu,Phe, 0.767; Gly,Arg,Pro,Met, 0.767; Glu,Cit,Arg,Met, 0.767; Arg,Val,Met,Ile, 0.767; Ala,Cit,Arg,Phe, 0.767; Gln,Arg,Val,Met, 0.767; Gln,Arg,Met,Lys, 0.766; Ser,Arg,Tyr,Met, 0.765; Arg,Val,Orn,Phe, 0.765; Arg,Pro,ABA,Met, 0.764; Gln,Arg,Met,Ile, 0.764; Asn,Gln,Arg,Phe, 0.764; Glu,Thr,Arg,Met, 0.763; Arg,Tyr,Met,Ile, 0.763; Ser,Arg,Met,Lys, 0.763; Thr,Arg,ABA,Met, 0.763; Thr,Arg,Met,Leu, 0.763; Arg,Pro,Tyr,Met, 0.763; Arg,ABA,Met,Orn, 0.763; Ser,Arg,Met,Leu, 0.763; Gln,Arg,Met,Orn, 0.763; Gln,Cit,Arg,Met, 0.763; Cit,Arg,Val,Phe, 0.763; Ser,Thr,Arg,Met, 0.762; Arg,Val,Phe,Trp, 0.762; Thr,Arg,Phe,Trp, 0.761; Arg,Met,Orn,Lys, 0.761; Thr,Arg,Pro,Met, 0.76; Arg,Pro,Phe,Trp, 0.759; Arg,ABA,Phe,Trp, 0.755; Gln,Arg,Pro,Met, 0.755; Arg,Tyr,Phe,Trp, 0.754; Arg,Orn,Lys,Phe, 0.753; Arg,Orn,Phe,Trp, 0.752; Arg,Lys,Phe,Trp, 0.751; Arg,Lys,Ile,Phe, 0.747; Arg,Tyr,Ile,Trp, 0.741

[104. Formulae with five explanatory variables obtained from amino acids before the start of treatment]

Ala,Arg,Met,Leu,Phe, 0.805; Ala,Arg,Met,Lys,Trp, 0.805; Ala,Arg,Val,Met,Phe, 0.803; Gly,Ala,Arg,Met,Ile, 0.8; Gln,Ala,Arg,Met,Phe, 0.796; Ala,Arg,Met,Phe,Trp, 0.796; Asn,Ala,Arg,Met,Trp, 0.796; Arg,Met,Lys,Phe,Trp, 0.796; Ser,Gly,Arg,Met,Trp, 0.796; Gly,Ala,Arg,Met,Trp, 0.795; Ala,Arg,Pro,Met,Trp, 0.795; Gln,His,Arg,Met,Trp, 0.795; Ser,Ala,Arg,Met,Trp, 0.794; Arg,Val,Met,Phe,Trp, 0.794; Glu,Gly,Ala,Arg,Met, 0.794; His,Cit,Arg,Met,Orn, 0.794; Ala,Arg,Met,Leu,Trp, 0.793; Arg,ABA,Met,Phe,Trp, 0.793; His,Cit,Arg,Met,Phe, 0.793; Ala,Arg,Met,Lys,Phe, 0.792; Ala,Cit,Arg,Met,Trp, 0.792; Gln,Ala,Arg,Met,Trp, 0.792; Arg,Met,Leu,Phe,Trp, 0.792; Cit,Arg,Met,Phe,Trp, 0.792; Gly,His,Arg,Met,Trp, 0.792; His,Ala,Arg,Met,Phe, 0.792; Ala,Arg,Val,Met,Lys, 0.792; His,Ala,Arg,Met,Trp, 0.791; Gln,Arg,Met,Phe,Trp, 0.791; Ala,Arg,Tyr,Val,Met, 0.791; Gln,Ala,Arg,Met,Leu, 0.79; Ala,Cit,Arg,Met,Ile, 0.79; Asn,Ala,Arg,Met,Phe, 0.789; Ala,Arg,Tyr,Met,Lys, 0.789; Asn,His,Arg,Tyr,Met, 0.789; Glu,Gly,Arg,Val,Met, 0.789; Ser,Arg,Met,Phe,Trp, 0.788; Ala,Arg,Met,Ile,Leu, 0.788; Thr,Ala,Arg,ABA,Met, 0.788; Ser,Ala,Arg,Met,Leu, 0.788; Glu,His,Arg,Met,Ile, 0.788; Thr,Ala,Arg,Met,Phe, 0.787; Ser,Ala,Arg,Met,Phe, 0.787; Glu,His,Arg,Met,Phe, 0.787; His,Arg,ABA,Met,Leu, 0.787; Ser,Ala,Arg,Met,Ile, 0.786; Arg,ABA,Val,Met,Trp, 0.786; Ala,Arg,ABA,Met,Phe, 0.785; Ser,His,Ala,Arg,Met, 0.785; His,Ala,Cit,Arg,Met, 0.785; Gly,Gln,Arg,Val,Met, 0.785; Glu,Ser,His,Arg,Met, 0.784; Asn,Arg,Pro,Met,Trp, 0.783; Arg,Met,Orn,Lys,Trp, 0.783; Glu,His,Arg,Met,Lys, 0.783; Arg,Val,Ile,Phe,Trp, 0.782; Asn,Arg,Val,Met,Trp, 0.782; Gln,Arg,Met,Leu,Trp, 0.782; His,Ala,Arg,Pro,Met, 0.782; Ser,Gln,Ala,Arg,Phe, 0.782; His,Arg,Met,Orn,Leu, 0.782; Arg,Tyr,Met,Leu,Trp, 0.78; Gln,Arg,Met,Orn,Phe, 0.779; Gln,Arg,Met,Ile,Phe, 0.779; Cit,Arg,Val,Ile,Phe, 0.778; His,Thr,Arg,Val,Met, 0.778; Glu,Arg,Tyr,Met,Phe, 0.778; Asn,His,Arg,Pro,Met, 0.778; Gly,Arg,Tyr,Val,Met, 0.778; Glu,Arg,Met,Orn,Phe, 0.778; Ala,Arg,Pro,Val,Phe, 0.777; Asn,Arg,Tyr,Val,Met, 0.777; Asn,Gly,Arg,ABA,Met, 0.776; Ser,Arg,Ile,Phe,Trp, 0.775; Ala,Arg,Orn,Ile,Phe, 0.775; Arg,Pro,Ile,Leu,Phe, 0.774; Gln,Ala,Arg,Val,Phe, 0.773; Asn,Thr,Arg,Met,Orn, 0.773; Ser,Thr,Arg,Met,Phe, 0.773; Thr,Ala,Arg,Phe,Trp, 0.772; Ala,Arg,Pro,Phe,Trp, 0.772; Gly,His,Thr,Arg,Met, 0.771; Asn,Thr,Arg,Met,Ile, 0.771; Thr,Arg,ABA,Met,Leu, 0.77; Ala,Arg,Orn,Lys,Phe, 0.769; Thr,Arg,Lys,Phe,Trp, 0.769; Asn,Ala,Arg,Val,Phe, 0.769; Glu,Asn,Thr,Arg,Met, 0.769; Arg,Tyr,Ile,Leu,Phe, 0.768; Thr,Cit,Arg,Met,Phe, 0.768; Glu,Thr,Arg,ABA,Met, 0.767; Ala,Arg,Lys,Phe,Trp, 0.766; Asn,Ala,Arg,Lys,Phe, 0.765; Thr,Arg,ABA,Met,Orn, 0.765; Arg,Val,Lys,Phe,Trp, 0.761; Thr,Arg,Tyr,Phe,Trp, 0.761; Thr,Arg,Val,Met,Lys, 0.76; Thr,Ala,Arg,Lys,Phe, 0.758; Arg,Orn,Lys,Phe,Trp, 0.753; His,Arg,Lys,Phe,Trp, 0.752

[105. Formulae with six explanatory variables obtained from amino acids before the start of treatment]

Glu,Ala,Arg,Met,Leu,Phe, 0.821; His,Arg,Val,Met,Lys,Phe, 0.82; Gly,Ala,Arg,Val,Met,Phe, 0.817; Glu,Ala,Arg,Val,Met,Phe, 0.813; Glu,Asn,Ala,Arg,Met,Trp, 0.811; Gly,Ala,Arg,Val,Met,Lys, 0.81; Ala,Arg,Val,Met,Lys,Phe, 0.809; Ala,Arg,Val,Met,Orn,Phe, 0.809; Gly,His,Ala,Arg,Met,Leu, 0.809; Ala,Arg,Val,Met,Phe,Trp, 0.808; Glu,Ala,Cit,Arg,Met,Trp, 0.806; Glu,Ala,Arg,Tyr,Met,Trp, 0.806; Glu,Ala,Arg,Met,Phe,Trp, 0.804; Gly,Ala,Arg,Met,Phe,Trp, 0.803; Ser,Ala,Arg,Val,Met,Phe, 0.803; Gly,Ala,Cit,Arg,Val,Met, 0.803; Thr,Ala,Arg,Met,Phe,Trp, 0.802; Glu,Ala,Arg,Pro,Met,Phe, 0.802; Ala,Arg,Met,Ile,Phe,Trp, 0.801; Glu,Ser,Ala,Arg,Met,Trp, 0.801; Gly,Gln,Ala,Arg,Met,Lys, 0.801; Gly,Ala,Arg,Met,Leu,Trp, 0.8; Glu,Arg,Met,Orn,Phe,Trp, 0.8; Ala,Arg,Pro,Ile,Leu,Phe, 0.8; Gln,Ala,Arg,Met,Phe,Trp, 0.799; Ser,Ala,Arg,Met,Phe,Trp, 0.799; Glu,Asn,Arg,Met,Phe,Trp, 0.799; Glu,Arg,Tyr,Met,Orn,Trp, 0.799; Glu,Gln,Arg,Met,Lys,Trp, 0.799; Gly,Ala,Arg,Pro,Met,Trp, 0.798; Glu,Ser,Ala,Arg,Met,Phe, 0.798; Glu,Gln,His,Arg,Met,Trp, 0.798; His,Ala,Arg,Met,Phe,Trp, 0.797; Gly,Gln,Ala,Arg,Met,Trp, 0.797; Glu,Arg,Met,Ile,Phe,Trp, 0.797; Glu,Ala,Arg,ABA,Met,Orn, 0.797; Arg,ABA,Met,Leu,Phe,Trp, 0.796; Glu,Arg,ABA,Met,Orn,Trp, 0.796; Ala,Arg,ABA,Met,Lys,Phe, 0.796; Gly,Gln,Thr,Ala,Arg,Met, 0.796; Ala,Arg,Val,Met,Lys,Leu, 0.796; Ser,Ala,Arg,Met,Lys,Trp, 0.795; Ala,Cit,Arg,Val,Met,Trp, 0.795; Gly,Gln,Arg,Met,Phe,Trp, 0.795; Ser,Ala,Arg,Val,Met,Trp, 0.795; Arg,Pro,Met,Lys,Phe,Trp, 0.795; Glu,Asn,Gly,Arg,Met,Trp, 0.795; Glu,Asn,Ala,Arg,Met,Ile, 0.795; Ser,Ala,Arg,Val,Met,Lys, 0.795; Gln,Ala,Arg,Met,Lys,Leu, 0.795; Ala,Arg,Val,Met,Lys,Ile, 0.795; Ala,Arg,Tyr,Met,Phe,Trp, 0.794; Gly,Ala,Cit,Arg,Met,Trp, 0.794; Gly,Thr,Ala,Arg,Met,Ile, 0.794; Ala,Arg,Pro,Val,Met,Trp, 0.794; Gly,Thr,Ala,Arg,Tyr,Met, 0.794; His,Arg,Tyr,Met,Lys,Trp, 0.794; Glu,Ser,Arg,Met,Ile,Trp, 0.794; Glu,Ala,Arg,Met,Lys,Phe, 0.793; Ala,Cit,Arg,Pro,Met,Trp, 0.793; Glu,His,Ala,Arg,Met,Phe, 0.793; Gln,Ala,Arg,Val,Ile,Phe, 0.793; Glu,Asn,Ala,Arg,ABA,Met, 0.793; Thr,Cit,Arg,Met,Phe,Trp, 0.792; Ala,Cit,Arg,Val,Met,Lys, 0.792; Asn,Ala,Arg,Met,Ile,Phe, 0.791; Gln,Ala,Arg,Met,Ile,Trp, 0.791; Ala,Arg,Tyr,Val,Met,Lys, 0.791; His,Ala,Arg,Val,Met,Trp, 0.79; Gln,Arg,Met,Orn,Phe,Trp, 0.79; Arg,Pro,ABA,Met,Phe,Trp, 0.79; His,Thr,Ala,Arg,Tyr,Met, 0.789; Gln,Thr,Ala,Arg,Met,Trp, 0.788; Asn,Gln,Arg,ABA,Met,Trp, 0.788; Ser,His,Arg,Met,Ile,Trp, 0.788; Glu,Ser,Ala,Cit,Arg,Met, 0.788; Thr,Arg,Met,Ile,Phe,Trp, 0.788; Gln,His,Thr,Ala,Arg,Met, 0.788; Thr,Ala,Arg,Pro,Tyr,Met, 0.788; Asn,Gln,Ala,Arg,Met,Leu, 0.788; Thr,Ala,Arg,Tyr,Met,Ile, 0.787; Ser,Ala,Arg,Val,Lys,Phe, 0.787; Gln,Thr,Ala,Arg,Met,Ile, 0.786; Thr,Ala,Arg,Tyr,Met,Lys, 0.786; Asn,Arg,ABA,Tyr,Met,Trp, 0.786; Ser,Ala,Arg,Val,Met,Ile, 0.786; Ala,Arg,ABA,Val,Lys,Phe, 0.785; His,Arg,Tyr,Val,Met,Trp, 0.785; Thr,Ala,Arg,Val,Lys,Phe, 0.784; Ala,Arg,Val,Lys,Leu,Phe, 0.784; Asn,Gln,Ala,Arg,Met,Orn, 0.784; Asn,Thr,Arg,Pro,Met,Trp, 0.783; Ser,Asn,Arg,Val,Met,Trp, 0.782; Asn,Gly,Arg,Pro,Met,Trp, 0.781; Ala,Arg,Val,Lys,Phe,Trp, 0.78; Gly,Ala,Arg,Val,Lys,Phe, 0.78; Ser,Asn,Thr,Arg,Met,Trp, 0.778; Thr,Arg,Tyr,Met,Ile,Trp, 0.778; Thr,Arg,Pro,Met,Ile,Trp, 0.774; Ser,Arg,Pro,Met,Lys,Trp, 0.773

[106. Formulae with two explanatory variables obtained from amino acids and amino acid-related metabolites before the start of treatment]

Arg,Kyn, 0.771; Arg,hKyn, 0.741; Cit,QA, 0.738; Arg,Lys, 0.737; His,QA, 0.736; Arg,QA, 0.735; hKyn,AnthA, 0.731; His,hKyn, 0.729; Gly,Arg, 0.729; Arg,hAnthA, 0.728; His,AnthA, 0.726; Cit,hKyn, 0.722; His,Kyn, 0.722; Arg,NP, 0.721; Thr,Arg, 0.72; AnthA,Kyn, 0.72; Trp,hKyn, 0.719; AnthA,QA, 0.716; Arg,AnthA, 0.715; Cit,Kyn, 0.713; hTrp,AnthA, 0.712; His,hTrp, 0.712; His,hAnthA, 0.711; Cit,NP, 0.71; Ala,hKyn, 0.709; Ala,Kyn, 0.708; Ala,AnthA, 0.707; Asn,Arg, 0.706; Arg,hTrp, 0.704; Arg,Trp, 0.703; Ser,Arg, 0.702; Gly,His, 0.701; Lys,hKyn, 0.7; His,Arg, 0.699; AnthA,NP, 0.699; Ala,Arg, 0.697; Cit,Arg, 0.697; Thr,hKyn, 0.697; His,NP, 0.697; Arg,Orn, 0.696; Ala,QA, 0.695; Gly,Cit, 0.695; Trp,Kyn, 0.694; Gly,Trp, 0.691; His,Orn, 0.691; Trp,hAnthA, 0.689; Gly,hKyn, 0.689; hKyn,hTrp, 0.689; Orn,hKyn, 0.689; hKyn,NP, 0.688; Gly,Ala, 0.687; His,Cit, 0.687; Ala,Cit, 0.686; Cit,hTrp, 0.686; Cit,hAnthA, 0.685; Cit,AnthA, 0.684; Trp,AnthA, 0.683; Asn,hKyn, 0.681; Cit,Trp, 0.68; Lys,AnthA, 0.68; Ser,hKyn, 0.679; His,Thr, 0.679; Gly,AnthA, 0.679; Ala,hAnthA, 0.678; Ser,Cit, 0.678; hTrp,QA, 0.677; Thr,QA, 0.676; hKyn,QA, 0.673; His,Trp, 0.673; Trp,QA, 0.673; Gly,QA, 0.671; hAnthA,AnthA, 0.671; Asn,AnthA, 0.671; Orn,NP, 0.67; Ala,NP, 0.669; Thr,Kyn, 0.669; Ser,AnthA, 0.668; hTrp,NP, 0.668; Ala,Orn, 0.667; hKyn,hAnthA, 0.667; Ala,hTrp, 0.664; hKyn,Kyn, 0.664; Ser,Ala, 0.661; Thr,Cit, 0.661; Thr,AnthA, 0.659; Gly,NP, 0.658; Ser,Trp, 0.657; Thr,hAnthA, 0.654; Lys,hAnthA, 0.654; Ala,Trp, 0.653; Trp,hTrp, 0.651; Thr,NP, 0.649; Orn,Trp, 0.648; Thr,Ala, 0.647; Cit,Lys, 0.645; Thr,Trp, 0.642; Lys,Trp, 0.641; Asn,Trp, 0.636; Thr,hTrp, 0.631; hAnthA,NP, 0.62

[107. Formulae with three explanatory variables obtained from amino acids and amino acid-related metabolites before the start of treatment]

Thr,Arg,Kyn, 0.771; Arg,Kyn,NP, 0.771; Arg,Lys,Kyn, 0.77; Arg,AnthA,Kyn, 0.769; His,Arg,Kyn, 0.769; Asn,Arg,Kyn, 0.769; Arg,hTrp,Kyn, 0.768; Gly,Arg,Kyn, 0.767; Arg,Orn,Kyn, 0.767; Ala,Arg,Kyn, 0.766; Arg,Kyn,QA, 0.766; Ser,Arg,Kyn, 0.763; Thr,Arg,QA, 0.763; Arg,hKyn,Kyn, 0.761; Ala,hKyn,AnthA, 0.76; Arg,hAnthA,Kyn, 0.757; Arg,Lys,QA, 0.757; Trp,hKyn,AnthA, 0.755; Arg,hKyn,AnthA, 0.754; Arg,hAnthA,QA, 0.753; Arg,hTrp,QA, 0.75; Gly,Thr,Arg, 0.748; Arg,Lys,hKyn, 0.747; Asn,Arg,QA, 0.746; Thr,Arg,hKyn, 0.746; Cit,AnthA,QA, 0.746; Arg,hKyn,QA, 0.745; Arg,Trp,QA, 0.745; Gly,Arg,hKyn, 0.744; Ala,Cit,Kyn, 0.744; Arg,AnthA,QA, 0.743; Arg,hKyn,hAnthA, 0.742; Arg,Lys,NP, 0.742; Arg,hKyn,hTrp, 0.741; Arg,Orn,hKyn, 0.741; Arg,hKyn,NP, 0.741; His,Arg,hKyn, 0.741; Ala,Arg,hAnthA, 0.74; Cit,hKyn,AnthA, 0.74; Arg,Lys,hTrp, 0.74; Cit,hAnthA,QA, 0.74; Arg,Lys,AnthA, 0.739; Cit,Arg,hKyn, 0.739; Cit,Arg,QA, 0.739; Arg,Lys,Trp, 0.739; His,Arg,Lys, 0.738; Arg,hAnthA,AnthA, 0.738; Thr,Arg,hAnthA, 0.738; Ser,Arg,QA, 0.737; Ala,Arg,QA, 0.737; Arg,Orn,QA, 0.737; Thr,Arg,Lys, 0.737; His,Arg,QA, 0.736; Arg,Orn,Lys, 0.736; Arg,hAnthA,NP, 0.735; Arg,QA,NP, 0.735; Thr,Arg,NP, 0.735; Ser,Arg,Lys, 0.735; Ser,Arg,hAnthA, 0.734; Asn,Gly,Arg, 0.734; Asn,Arg,Lys, 0.734; Cit,hKyn,QA, 0.733; Arg,hAnthA,hTrp, 0.732; Gly,Arg,AnthA, 0.732; Arg,AnthA,NP, 0.731; Gly,Arg,NP, 0.731; Gly,Arg,hAnthA, 0.73; Gly,Arg,hTrp, 0.73; Arg,Orn,hAnthA, 0.729; Thr,Arg,Trp, 0.729; Asn,Arg,hAnthA, 0.729; Ser,Gly,Arg, 0.729; Asn,Arg,AnthA, 0.728; Gly,Arg,Trp, 0.728; Thr,Arg,hTrp, 0.728; Gly,His,Arg, 0.728; Arg,hTrp,AnthA, 0.727; Thr,hKyn,AnthA, 0.727; Gly,Arg,Orn, 0.727; Ala,hAnthA,AnthA, 0.725; Cit,Arg,hAnthA, 0.724; Arg,hTrp,NP, 0.724; Gly,Ala,Arg, 0.723; Ala,Arg,AnthA, 0.721; Ser,Arg,NP, 0.721; Asn,Arg,NP, 0.72; Arg,Orn,AnthA, 0.717; Cit,Arg,AnthA, 0.717; Arg,Trp,NP, 0.717; Ser,Arg,AnthA, 0.715; His,Arg,AnthA, 0.715; Asn,Arg,hTrp, 0.714; Cit,Trp,hKyn, 0.714; His,Arg,hTrp, 0.711; Asn,Ala,Arg, 0.709; Ala,Arg,hTrp, 0.709; Arg,Trp,hTrp, 0.706; Asn,Arg,Trp, 0.704; Arg,Orn,hTrp, 0.702; Cit,Trp,hAnthA, 0.696

[108. Formulae with four explanatory variables obtained from amino acids and amino acid-related metabolites before the start of treatment]

Ser,Gly,Arg,QA, 0.775; Thr,Arg,hTrp,Kyn, 0.773; His,Arg,AnthA,Kyn, 0.771; Ala,Arg,Lys,Kyn, 0.769; Ala,Arg,Lys,QA, 0.769; Ser,Arg,hTrp,Kyn, 0.768; Arg,Orn,hTrp,Kyn, 0.768; Thr,Arg,Lys,Kyn, 0.767; Ala,Arg,Orn,Kyn, 0.767; Asn,Gly,Arg,Kyn, 0.767; Ala,Arg,hAnthA,Kyn, 0.766; Arg,AnthA,Kyn,QA, 0.766; Asn,Arg,Trp,Kyn, 0.766; Thr,Arg,Kyn,QA, 0.766; Arg,hKyn,Kyn,NP, 0.766; Ala,Arg,Trp,Kyn, 0.766; Thr,Arg,Lys,QA, 0.765; Ser,Arg,hAnthA,QA, 0.764; Arg,Lys,AnthA,Kyn, 0.764; Thr,Arg,hAnthA,Kyn, 0.764; His,Arg,hTrp,Kyn, 0.764; Ser,Ala,Arg,Kyn, 0.764; Ser,Arg,AnthA,Kyn, 0.764; Arg,hKyn,hTrp,Kyn, 0.763; Arg,Orn,Lys,Kyn, 0.763; His,Arg,hKyn,Kyn, 0.763; Arg,hAnthA,hTrp,Kyn, 0.763; Arg,Lys,hKyn,Kyn, 0.763; Thr,Arg,hKyn,AnthA, 0.763; Ser,Arg,hAnthA,Kyn, 0.762; Arg,Lys,hAnthA,Kyn, 0.762; Gly,Arg,hKyn,AnthA, 0.761; Asn,Arg,hKyn,Kyn, 0.761; Gly,Cit,Arg,QA, 0.76; His,Arg,Lys,NP, 0.76; Arg,Lys,hKyn,AnthA, 0.759; Arg,Lys,AnthA,QA, 0.759; Cit,Arg,Lys,QA, 0.759; Ser,Arg,Kyn,QA, 0.759; Ala,Arg,Lys,AnthA, 0.758; Ala,Cit,hTrp,Kyn, 0.758; Thr,Arg,hKyn,hTrp, 0.758; Arg,Orn,hKyn,Kyn, 0.758; Arg,hAnthA,hTrp,QA, 0.757; Arg,Lys,hAnthA,QA, 0.757; Gly,Arg,hAnthA,Kyn, 0.757; Arg,hKyn,hAnthA,AnthA, 0.756; Ser,Arg,hKyn,AnthA, 0.756; Ser,Gly,Arg,hKyn, 0.756; Arg,hKyn,hTrp,AnthA, 0.755; Asn,Thr,Arg,QA, 0.755; Thr,Arg,hAnthA,NP, 0.754; His,Arg,hAnthA,Kyn, 0.754; Arg,hAnthA,AnthA,QA, 0.753; Arg,hTrp,AnthA,QA, 0.753; Arg,Lys,hKyn,hTrp, 0.752; Arg,hAnthA,Kyn,NP, 0.752; Gly,Arg,hKyn,hTrp, 0.752; Asn,Arg,hAnthA,QA, 0.751; Asn,Arg,hKyn,QA, 0.751; Arg,Trp,AnthA,QA, 0.751; Cit,Arg,hAnthA,Kyn, 0.75; Ser,Arg,hKyn,hAnthA, 0.75; Arg,Trp,hKyn,AnthA, 0.75; Asn,His,Arg,QA, 0.75; Cit,Arg,AnthA,Kyn, 0.749; Arg,Orn,Lys,hKyn, 0.748; His,Arg,AnthA,QA, 0.748; Ser,Arg,Lys,hKyn, 0.747; Asn,Ala,Arg,hKyn, 0.747; Ser,Cit,Arg,Kyn, 0.747; Arg,Lys,hAnthA,AnthA, 0.746; Arg,hKyn,hAnthA,hTrp, 0.746; His,Arg,hKyn,hTrp, 0.746; His,Cit,Arg,Kyn, 0.746; Ser,Asn,Arg,QA, 0.746; Ser,Arg,hAnthA,NP, 0.745; Arg,hKyn,hTrp,NP, 0.745; Ser,Cit,Arg,QA, 0.745; Cit,Arg,hKyn,QA, 0.744; Arg,AnthA,QA,NP, 0.744; Ala,Arg,AnthA,QA, 0.744; Asn,Arg,Lys,hKyn, 0.743; Ala,Arg,hAnthA,hTrp, 0.743; Ala,Arg,hAnthA,NP, 0.743; Ser,Gly,Arg,hAnthA, 0.743; Cit,Arg,AnthA,QA, 0.742; Cit,Arg,hKyn,hAnthA, 0.742; Ser,Cit,Arg,hKyn, 0.742; Gly,Ala,Arg,hAnthA, 0.742; Ser,Thr,Arg,hKyn, 0.742; Ser,Arg,Orn,hKyn, 0.741; Asn,Arg,hAnthA,AnthA, 0.74; Arg,Trp,hKyn,hTrp, 0.74; Arg,Lys,hAnthA,hTrp, 0.739; Asn,Arg,Trp,hAnthA, 0.735; Asn,Arg,hAnthA,hTrp, 0.735; Ser,Asn,Arg,hAnthA, 0.734; Ser,His,Arg,hAnthA, 0.734; Cit,Trp,hAnthA,AnthA, 0.713

[109. Formulae with five explanatory variables obtained from amino acids and amino acid-related metabolites before the start of treatment]

Ser,Gly,Thr,Arg,QA, 0.797; Ser,Gly,Cit,Arg,QA, 0.782; Gly,Thr,Arg,Orn,QA, 0.779; Ser,Gly,Arg,hAnthA,QA, 0.777; Asn,Ala,Arg,Lys,Kyn, 0.777; Ser,Gly,Ala,Arg,QA, 0.776; Thr,Ala,Arg,hAnthA,Kyn, 0.775; Asn,Ala,Arg,AnthA,Kyn, 0.773; Arg,hKyn,hTrp,AnthA,Kyn, 0.773; Ala,Arg,hTrp,Kyn,NP, 0.773; Thr,Arg,hTrp,AnthA,QA, 0.772; Thr,Ala,Arg,AnthA,QA, 0.772; Ser,Arg,hAnthA,hTrp,Kyn, 0.771; Ser,Cit,Arg,hAnthA,QA, 0.771; Arg,hTrp,AnthA,Kyn,NP, 0.771; Gly,Ala,Arg,hTrp,Kyn, 0.77; Ala,Arg,hAnthA,Kyn,NP, 0.768; Asn,Arg,Lys,AnthA,Kyn, 0.768; Ser,Arg,hAnthA,Kyn,QA, 0.767; Ser,His,Arg,hAnthA,QA, 0.767; Ser,Arg,Orn,hAnthA,QA, 0.767; Ala,Cit,hAnthA,hTrp,Kyn, 0.766; Arg,Lys,hTrp,AnthA,QA, 0.766; Gly,Arg,hAnthA,hTrp,QA, 0.766; Arg,Lys,hAnthA,hTrp,Kyn, 0.765; Cit,Arg,Trp,hTrp,Kyn, 0.765; Cit,Arg,Trp,hTrp,QA, 0.765; Ser,Arg,hKyn,hAnthA,QA, 0.764; Ala,Arg,Trp,hAnthA,Kyn, 0.764; Ala,Cit,hAnthA,AnthA,QA, 0.764; Asn,His,Arg,AnthA,Kyn, 0.764; Arg,hAnthA,hTrp,AnthA,Kyn, 0.763; Asn,Ala,Arg,hKyn,AnthA, 0.763; Ala,Arg,hAnthA,Kyn,QA, 0.763; Arg,Orn,Lys,AnthA,QA, 0.763; Asn,Arg,hKyn,hAnthA,QA, 0.763; Ser,Arg,Lys,hKyn,AnthA, 0.763; Ser,Gly,Arg,hAnthA,Kyn, 0.763; Arg,Lys,Trp,AnthA,Kyn, 0.763; Ala,Arg,Lys,AnthA,NP, 0.763; Ser,Arg,Lys,AnthA,Kyn, 0.763; Gly,Ala,Arg,Lys,hKyn, 0.762; His,Ala,Arg,Lys,hKyn, 0.762; Thr,Arg,hAnthA,QA,NP, 0.762; Thr,Arg,hAnthA,Kyn,NP, 0.762; Ser,Arg,Lys,hAnthA,QA, 0.761; Cit,Arg,Orn,hTrp,Kyn, 0.761; Thr,Arg,Trp,hAnthA,QA, 0.761; Arg,Lys,hAnthA,hTrp,QA, 0.76; Asn,Arg,hAnthA,hTrp,Kyn, 0.76; Ala,Arg,Orn,Lys,hKyn, 0.76; Arg,Trp,hKyn,hAnthA,Kyn, 0.76; His,Thr,Arg,hAnthA,Kyn, 0.76; Thr,Ala,Arg,Lys,hAnthA, 0.76; Arg,Lys,Trp,hKyn,AnthA, 0.759; Cit,Arg,Lys,hKyn,QA, 0.759; Gly,Arg,hAnthA,Kyn,QA, 0.759; Gly,Arg,Lys,hKyn,Kyn, 0.759; Ser,Gly,Arg,Trp,Kyn, 0.759; Ser,Arg,hAnthA,AnthA,NP, 0.758; Ser,Gly,Arg,hAnthA,NP, 0.758; Ser,Asn,Arg,AnthA,QA, 0.758; Ala,Cit,Arg,Kyn,QA, 0.758; Ser,Arg,Orn,hAnthA,Kyn, 0.757; Arg,Trp,hAnthA,AnthA,QA, 0.756; Thr,Ala,Arg,hAnthA,NP, 0.756; Arg,Lys,hKyn,hAnthA,hTrp, 0.755; His,Arg,hKyn,hTrp,AnthA, 0.755; Asn,Gly,Arg,hAnthA,QA, 0.754; Arg,hKyn,hAnthA,AnthA,NP, 0.754; Gly,Arg,Lys,hAnthA,QA, 0.754; Ser,Arg,hKyn,hTrp,QA, 0.754; Arg,Orn,hKyn,hAnthA,AnthA, 0.754; Ser,Arg,Lys,hKyn,hTrp, 0.754; Arg,hAnthA,AnthA,Kyn,NP, 0.753; Arg,Lys,Trp,hKyn,QA, 0.753; Ala,Arg,Orn,hAnthA,QA, 0.753; Ser,His,Arg,hAnthA,NP, 0.752; Asn,Arg,Trp,hAnthA,Kyn, 0.751; Asn,Arg,Lys,hAnthA,QA, 0.751; Cit,Arg,hKyn,hAnthA,hTrp, 0.751; Ser,Cit,Arg,hTrp,QA, 0.751; Arg,Orn,Lys,hKyn,QA, 0.751; Thr,Arg,Lys,hAnthA,NP, 0.751; Asn,Ala,Arg,hAnthA,hTrp, 0.75; Ser,His,Arg,Lys,hKyn, 0.75; Ser,Arg,Lys,hKyn,hAnthA, 0.749; Arg,Lys,hKyn,hAnthA,QA, 0.748; Arg,Lys,Trp,hKyn,hTrp, 0.748; Ala,Arg,Trp,hAnthA,AnthA, 0.747; Ser,Ala,Arg,hAnthA,hTrp, 0.747; Ser,Arg,Orn,hAnthA,AnthA, 0.747; Ser,Gly,Arg,hKyn,hAnthA, 0.746; Ser,Gly,Arg,Trp,hAnthA, 0.746; Ser,Gly,Arg,hAnthA,hTrp, 0.746; Arg,Lys,Trp,hKyn,hAnthA, 0.746; Asn,Thr,Ala,Arg,hAnthA, 0.745; Ser,Asn,Ala,Arg,hAnthA, 0.738; Asn,Arg,Trp,hKyn,hAnthA, 0.738; Cit,Trp,hKyn,hAnthA,AnthA, 0.738

[110. Formulae with six explanatory variables obtained from amino acids and amino acid-related metabolites before the start of treatment]

Ser,Asn,Gly,His,Arg,QA, 0.804; Ser,Asn,Gly,Arg,hTrp,QA, 0.796; Ser,Gly,Arg,Lys,hTrp,QA, 0.795; Ser,Asn,Gly,Thr,Arg,QA, 0.793; Asn,Gly,Thr,Ala,Arg,QA, 0.791; Thr,Ala,Arg,Lys,AnthA,QA, 0.79; Asn,Gly,Thr,Cit,Arg,QA, 0.789; Ser,Gly,Thr,Arg,hAnthA,QA, 0.788; Asn,Thr,Ala,Arg,hTrp,Kyn, 0.788; Ser,Asn,Ala,Arg,hAnthA,Kyn, 0.786; Gly,Thr,Arg,hAnthA,hTrp,QA, 0.786; Ser,Asn,Gly,Arg,Kyn,QA, 0.786; Ser,Gly,Arg,hAnthA,hTrp,QA, 0.785; Ser,Asn,Gly,Arg,QA,NP, 0.785; Ser,Asn,Gly,Arg,hAnthA,QA, 0.783; Ser,Gly,Ala,Arg,Kyn,QA, 0.783; Ser,Gly,His,Arg,Lys,QA, 0.783; Ser,Gly,Arg,Orn,Lys,QA, 0.783; Ser,Gly,His,Arg,hTrp,QA, 0.782; Ser,Ala,Arg,hAnthA,Kyn,QA, 0.781; Thr,Arg,Lys,Trp,AnthA,Kyn, 0.781; Ser,Gly,Cit,Arg,hAnthA,QA, 0.78; Ser,Arg,hKyn,hAnthA,hTrp,QA, 0.78; Asn,Ala,Arg,Lys,AnthA,Kyn, 0.779; Ser,Gly,Ala,Arg,hAnthA,QA, 0.779; Gly,Thr,Arg,hAnthA,AnthA,QA, 0.779; Asn,Thr,Arg,hTrp,AnthA,QA, 0.779; Ser,Gly,Arg,hKyn,hAnthA,QA, 0.779; Ser,Gly,Arg,hAnthA,QA,NP, 0.779; Asn,Ala,Arg,hAnthA,AnthA,NP, 0.779; Asn,Ala,Arg,hAnthA,Kyn,NP, 0.779; Ser,Ala,Arg,hKyn,hAnthA,Kyn, 0.779; Asn,Gly,Ala,Cit,Arg,QA, 0.779; Asn,Arg,hTrp,AnthA,Kyn,QA, 0.779; Ser,Arg,hAnthA,hTrp,AnthA,QA, 0.778; Asn,Ala,Arg,AnthA,Kyn,NP, 0.778; Ser,Gly,Arg,Trp,hAnthA,QA, 0.777; Ala,Cit,Arg,Lys,hTrp,Kyn, 0.777; Ser,His,Arg,hAnthA,hTrp,QA, 0.777; Ser,His,Ala,Arg,hAnthA,Kyn, 0.777; Asn,Cit,Arg,hTrp,Kyn,QA, 0.777; Thr,Arg,hKyn,hTrp,AnthA,QA, 0.777; Ser,Gly,Arg,hAnthA,Kyn,QA, 0.776; Ser,Gly,Arg,Lys,hAnthA,QA, 0.776; Ser,Arg,Orn,hAnthA,hTrp,QA, 0.776; Asn,Ala,Arg,hTrp,AnthA,QA, 0.776; Ala,Cit,Arg,Lys,Kyn,QA, 0.776; Asn,Ala,Arg,Lys,AnthA,QA, 0.774; Thr,Ala,Arg,Lys,hAnthA,NP, 0.774; Ala,Arg,Lys,hAnthA,hTrp,Kyn, 0.773; Gly,Ala,Arg,Lys,Kyn,QA, 0.773; Ala,Cit,Arg,Lys,Kyn,NP, 0.773; Thr,Ala,Arg,Lys,hKyn,QA, 0.773; Asn,Cit,Arg,AnthA,Kyn,QA, 0.773; Asn,His,Arg,hAnthA,AnthA,Kyn, 0.772; Gly,Ala,Arg,Lys,hAnthA,Kyn, 0.771; Thr,Arg,Lys,AnthA,Kyn,QA, 0.771; Gly,Ala,Cit,Arg,Lys,Kyn, 0.771; Ser,Arg,Lys,hTrp,Kyn,QA, 0.771; Arg,Orn,Lys,Trp,AnthA,Kyn, 0.771; Ala,Cit,Arg,Lys,hAnthA,Kyn, 0.771; Ser,Arg,hAnthA,AnthA,Kyn,QA, 0.771; Asn,Ala,Arg,hKyn,AnthA,QA, 0.771; His,Thr,Arg,Lys,hTrp,QA, 0.768; Gly,His,Ala,Arg,Lys,Kyn, 0.767; His,Ala,Arg,Lys,AnthA,QA, 0.766; Asn,His,Cit,Arg,hAnthA,QA, 0.766; Arg,Orn,Lys,hKyn,AnthA,NP, 0.766; Arg,Lys,Trp,hKyn,hAnthA,AnthA, 0.765; Ser,Ala,Arg,hAnthA,AnthA,NP, 0.765; Ser,Ala,Arg,Orn,hAnthA,QA, 0.765; Thr,Ala,Cit,hAnthA,hTrp,Kyn, 0.765; Arg,Lys,hAnthA,hTrp,Kyn,NP, 0.765; Thr,Arg,Lys,hKyn,hAnthA,AnthA, 0.764; Ser,Arg,Orn,hAnthA,AnthA,Kyn, 0.764; His,Arg,hAnthA,hTrp,AnthA,QA, 0.763; Ser,Arg,Orn,hAnthA,hTrp,NP, 0.763; Asn,Arg,Trp,hKyn,AnthA,NP, 0.763; Arg,Lys,hAnthA,AnthA,Kyn,QA, 0.763; Cit,Arg,Lys,hKyn,hTrp,AnthA, 0.763; Asn,Ala,Arg,Lys,AnthA,NP, 0.763; Gly,Arg,Lys,hKyn,AnthA,NP, 0.763; Arg,Lys,Trp,hKyn,hTrp,AnthA, 0.762; Cit,Arg,hAnthA,hTrp,AnthA,Kyn, 0.762; Gly,Arg,hAnthA,hTrp,AnthA,QA, 0.762; Ala,Cit,Trp,hAnthA,hTrp,Kyn, 0.761; Thr,Ala,Arg,hAnthA,AnthA,NP, 0.761; His,Arg,Lys,hKyn,hTrp,AnthA, 0.76; Gly,Arg,Lys,Trp,hKyn,AnthA, 0.759; Arg,Orn,hAnthA,AnthA,Kyn,QA, 0.759; Arg,Lys,Trp,hAnthA,AnthA,Kyn, 0.758; Cit,Arg,hKyn,hAnthA,hTrp,QA, 0.757; Ala,Arg,Orn,hAnthA,AnthA,QA, 0.757; Ser,Cit,Arg,hAnthA,Kyn,NP, 0.756; Ser,Gly,Ala,Arg,hAnthA,NP, 0.756; Ala,Cit,Arg,hAnthA,hTrp,AnthA, 0.754; Ala,Arg,Orn,hAnthA,hTrp,AnthA, 0.754; Asn,Arg,Trp,hKyn,hAnthA,Kyn, 0.754; Asn,Thr,Arg,Trp,hAnthA,AnthA, 0.753; Asn,His,Arg,Lys,hKyn,AnthA, 0.752

[111. Formulae with two explanatory variables obtained from amino acids after the start of treatment]

Leu,Phe, 0.783; Orn,Trp, 0.783; Val,Phe, 0.767; Met,Phe, 0.758; Phe,Trp, 0.754; Val,Orn, 0.749; Ser,Val, 0.749; Pro,Val, 0.747; Asn,Val, 0.747; Ala,Val, 0.743; Cit,Val, 0.741; Val,Trp, 0.741; Gly,Val, 0.741; Tyr,Val, 0.741; Glu,Val, 0.741; Val,Met, 0.741; Arg,Val, 0.737; Cit,Trp, 0.737; Gln,Val, 0.737; Orn,Leu, 0.737; ABA,Val, 0.735; His,Phe, 0.735; Asn,Leu, 0.733; Arg,Leu, 0.73; Arg,Trp, 0.73; Val,Ile, 0.73; Ser,Leu, 0.73; His,Orn, 0.728; Thr,Cit, 0.726; Ile,Phe, 0.726; Leu,Trp, 0.726; Gly,Leu, 0.726; Cit,Leu, 0.724; Val,Lys, 0.72; Met,Orn, 0.72; Arg,Orn, 0.718; Arg,ABA, 0.718; Ile,Trp, 0.716; Gln,Leu, 0.716; Ala,Leu, 0.716; ABA,Leu, 0.716; Thr,Val, 0.715; His,Val, 0.715; Lys,Trp, 0.715; His,Leu, 0.715; Lys,Leu, 0.715; Thr,Trp, 0.713; Val,Leu, 0.713; Thr,Leu, 0.713; Thr,Orn, 0.711; Asn,Arg, 0.711; His,Cit, 0.711; Ile,Leu, 0.711; Glu,Leu, 0.711; Arg,Met, 0.709; Pro,Leu, 0.709; Cit,Tyr, 0.707; Tyr,Leu, 0.707; Thr,Phe, 0.705; His,Trp, 0.703; Asn,Trp, 0.703; Arg,Ile, 0.701; Cit,Arg, 0.701; Cit,Met, 0.701; Glu,Trp, 0.699; Pro,Trp, 0.699; His,Ile, 0.699; His,Thr, 0.698; His,Arg, 0.698; Met,Leu, 0.698; Gly,Trp, 0.696; Ser,Trp, 0.696; Gln,Trp, 0.696; Ala,Trp, 0.694; Met,Trp, 0.694; Thr,Ile, 0.694; ABA,Trp, 0.692; Tyr,Trp, 0.692; Thr,Ala, 0.692; Glu,Arg, 0.69; Arg,Phe, 0.69; Arg,Tyr, 0.69; Thr,Lys, 0.69; Thr,Met, 0.69; Arg,Pro, 0.69; Cit,ABA, 0.69; Gln,Thr, 0.688; Thr,Tyr, 0.688; Cit,Ile, 0.688; Thr,Pro, 0.686; Gln,Arg, 0.684; Ala,Arg, 0.682; Glu,Thr, 0.682; Thr,ABA, 0.682; Arg,Lys, 0.682; Asn,Thr, 0.681; Ser,Arg, 0.679; Gly,Thr, 0.679; Gly,Arg, 0.677; Ser,Thr, 0.669

[112. Formulae with three explanatory variables obtained from amino acids after the start of treatment]

Orn,Leu,Phe, 0.832; Cit,Leu,Phe, 0.813; Arg,Leu,Phe, 0.803; Pro,Leu,Phe, 0.802; Gly,Leu,Phe, 0.798; Ile,Leu,Phe, 0.796; Val,Leu,Phe, 0.794; Lys,Leu,Phe, 0.794; Val,Orn,Phe, 0.792; Orn,Leu,Trp, 0.792; Glu,Leu,Phe, 0.79; Gly,Orn,Trp, 0.79; Arg,Phe,Trp, 0.79; Pro,Val,Phe, 0.788; Ser,Leu,Phe, 0.788; Tyr,Orn,Trp, 0.788; His,Leu,Phe, 0.786; Thr,Leu,Phe, 0.786; Ala,Orn,Trp, 0.786; Cit,Orn,Trp, 0.786; Orn,Phe,Trp, 0.784; Gln,Orn,Trp, 0.784; Val,Phe,Trp, 0.783; Asn,Leu,Phe, 0.783; Met,Orn,Trp, 0.783; Arg,Orn,Trp, 0.783; Asn,Orn,Trp, 0.783; ABA,Orn,Trp, 0.783; Cit,Val,Phe, 0.781; ABA,Leu,Phe, 0.781; Pro,Orn,Trp, 0.781; Ser,Orn,Trp, 0.781; Arg,Val,Phe, 0.779; His,Orn,Trp, 0.779; Val,Met,Phe, 0.777; Cit,Phe,Trp, 0.777; Thr,Orn,Trp, 0.777; Tyr,Val,Phe, 0.773; Val,Lys,Phe, 0.771; Val,Orn,Trp, 0.771; Thr,Phe,Trp, 0.769; Glu,Val,Phe, 0.767; Ala,Val,Phe, 0.767; Ser,Val,Phe, 0.767; Asn,Val,Phe, 0.767; Gln,Phe,Trp, 0.767; Ser,Phe,Trp, 0.766; Lys,Phe,Trp, 0.764; Gln,Orn,Leu, 0.764; Val,Ile,Phe, 0.76; Arg,Val,Orn, 0.76; Pro,Val,Orn, 0.76; Arg,ABA,Phe, 0.76; Tyr,Phe,Trp, 0.758; Cit,Met,Phe, 0.758; Val,Orn,Lys, 0.758; His,Ile,Phe, 0.756; Tyr,Val,Orn, 0.756; Pro,Phe,Trp, 0.754; Asn,Phe,Trp, 0.754; His,Phe,Trp, 0.752; Cit,Val,Trp, 0.752; Thr,Val,Orn, 0.752; Gly,Cit,Val, 0.752; Arg,Pro,Val, 0.75; Val,Orn,Ile, 0.75; Gly,Val,Orn, 0.75; Arg,Orn,Leu, 0.749; Cit,Val,Orn, 0.749; Cit,Arg,Leu, 0.749; Glu,Cit,Val, 0.749; Ser,Cit,Val, 0.747; Val,Orn,Leu, 0.747; Ala,Val,Orn, 0.747; Ser,Val,Orn, 0.745; Cit,Val,Ile, 0.745; Arg,Val,Trp, 0.745; Ser,Cit,Leu, 0.743; Cit,Lys,Trp, 0.743; Cit,Pro,Trp, 0.743; Arg,Val,Lys, 0.741; Thr,Cit,Val, 0.741; Asn,Arg,Leu, 0.741; Thr,Cit,Phe, 0.737; Thr,Orn,Leu, 0.737; Arg,Val,Ile, 0.737; Arg,ABA,Orn, 0.737; Glu,Cit,Trp, 0.735; Gln,Cit,Trp, 0.733; Asn,Cit,Trp, 0.732; Arg,Val,Met, 0.73; Arg,Pro,Leu, 0.728; Arg,Lys,Leu, 0.726; Thr,Cit,Arg, 0.726; Gly,Arg,Leu, 0.724; Arg,Met,Leu, 0.724; Ser,Arg,Leu, 0.722; Glu,Arg,Val, 0.72; Glu,Arg,Trp, 0.72; Glu,Arg,Leu, 0.705

[113. Formulae with four explanatory variables obtained from amino acids after the start of treatment]

Gln,Orn,Leu,Phe, 0.834; Val,Orn,Leu,Phe, 0.832; Met,Orn,Leu,Phe, 0.83; Cit,Pro,Leu,Phe, 0.828; Arg,Orn,Leu,Phe, 0.828; Arg,Pro,Leu,Phe, 0.824; Cit,Orn,Leu,Phe, 0.82; Cit,Lys,Leu,Phe, 0.82; Cit,ABA,Leu,Phe, 0.817; Cit,Tyr,Leu,Phe, 0.815; Cit,Met,Leu,Phe, 0.813; Cit,Val,Leu,Phe, 0.813; Pro,Leu,Phe,Trp, 0.813; Pro,Lys,Leu,Phe, 0.811; Arg,Ile,Leu,Phe, 0.809; Cit,Ile,Leu,Phe, 0.809; Asn,Cit,Leu,Phe, 0.809; Glu,Cit,Leu,Phe, 0.809; Ala,Pro,Leu,Phe, 0.807; Arg,Leu,Phe,Trp, 0.805; Arg,ABA,Leu,Phe, 0.805; Pro,Ile,Leu,Phe, 0.805; Asn,Pro,Leu,Phe, 0.805; Pro,Tyr,Leu,Phe, 0.805; Pro,Val,Phe,Trp, 0.805; Gly,Orn,Phe,Trp, 0.803; Glu,Pro,Leu,Phe, 0.803; Val,Met,Orn,Phe, 0.803; Thr,Met,Leu,Phe, 0.803; Arg,Lys,Leu,Phe, 0.802; Gln,Arg,Leu,Phe, 0.802; Pro,Tyr,Val,Phe, 0.802; Val,Met,Leu,Phe, 0.802; Arg,Tyr,Leu,Phe, 0.8; Gln,Lys,Leu,Phe, 0.8; Ala,Met,Leu,Phe, 0.8; Gly,Met,Leu,Phe, 0.8; Asn,Arg,Leu,Phe, 0.798; Gln,Pro,Leu,Phe, 0.798; Gly,Pro,Leu,Phe, 0.798; Gln,Val,Leu,Phe, 0.798; Glu,Gly,Leu,Phe, 0.798; Gly,Lys,Leu,Phe, 0.798; Glu,Asn,Leu,Phe, 0.798; Gly,Ala,Leu,Phe, 0.798; Pro,Val,Orn,Phe, 0.796; Orn,Ile,Phe,Trp, 0.796; Gln,Ala,Leu,Phe, 0.796; Tyr,Val,Leu,Phe, 0.796; ABA,Lys,Leu,Phe, 0.796; Glu,Gln,Leu,Phe, 0.794; Tyr,Lys,Leu,Phe, 0.794; Ile,Leu,Phe,Trp, 0.792; Asn,Val,Orn,Phe, 0.792; His,Met,Leu,Phe, 0.792; Thr,Lys,Leu,Phe, 0.792; Tyr,Orn,Lys,Trp, 0.792; Ser,Orn,Phe,Trp, 0.79; Ser,Pro,Val,Phe, 0.79; Cit,Val,Orn,Phe, 0.79; Gly,Gln,Leu,Phe, 0.79; Gln,Val,Orn,Phe, 0.79; Cit,ABA,Val,Phe, 0.79; Glu,His,Leu,Phe, 0.79; Ala,Ile,Leu,Phe, 0.79; Cit,Pro,Val,Phe, 0.788; Val,Orn,Lys,Phe, 0.788; His,Arg,Val,Phe, 0.788; Gln,Val,Phe,Trp, 0.788; Tyr,Ile,Leu,Phe, 0.788; Ser,Lys,Leu,Phe, 0.788; Thr,Tyr,Leu,Phe, 0.788; Ser,Tyr,Val,Phe, 0.788; Lys,Ile,Leu,Phe, 0.786; His,Lys,Leu,Phe, 0.786; Gln,Orn,Lys,Trp, 0.786; Glu,Val,Phe,Trp, 0.784; Asn,Val,Phe,Trp, 0.784; ABA,Tyr,Leu,Phe, 0.784; Asn,ABA,Leu,Phe, 0.784; Tyr,Orn,Phe,Trp, 0.783; Orn,Lys,Leu,Trp, 0.783; Glu,Asn,Val,Phe, 0.783; Asn,Tyr,Leu,Phe, 0.781; Cit,Val,Lys,Phe, 0.779; Ala,Arg,Val,Phe, 0.779; Arg,Val,Met,Phe, 0.779; His,Tyr,Val,Phe, 0.779; Gln,Tyr,Val,Phe, 0.779; Asn,Pro,Val,Phe, 0.777; Ser,Val,Met,Phe, 0.777; Gly,Val,Met,Phe, 0.775; Gly,Thr,Val,Phe, 0.773; Ser,Thr,Val,Phe, 0.773; Val,Met,Ile,Phe, 0.773; Gln,Thr,Val,Phe, 0.773; His,Val,Lys,Phe, 0.769; Thr,Ala,Val,Phe, 0.767; Gly,Val,Lys,Phe, 0.76; Ala,Val,Ile,Phe, 0.76

[114. Formulae with five explanatory variables obtained from amino acids after the start of treatment]

Arg,Orn,Leu,Phe,Trp, 0.853; Gln,Pro,Orn,Leu,Phe, 0.847; Thr,Arg,Pro,Leu,Phe, 0.843; Pro,Orn,Leu,Phe,Trp, 0.839; Pro, Tyr, Orn, Leu, Phe, 0.839; Ser, Pro, Orn, Leu, Phe, 0.839; Val,Orn,Leu,Phe,Trp, 0.837; Pro,Val,Orn,Leu,Phe, 0.837; Asn, Pro, Orn, Leu, Phe, 0.837; Pro, Orn, Lys, Leu, Phe, 0.836; Gln,His,Orn,Leu,Phe, 0.836; Ala,Arg,Orn,Leu,Phe, 0.834; Ser,Thr,Orn,Leu,Phe, 0.834; Cit,Pro,Orn,Leu,Phe, 0.832; Ala,Orn,Ile,Leu,Phe, 0.832; Arg,Pro,Orn,Leu,Phe, 0.83; Thr,Arg,Orn,Leu,Phe, 0.83; Arg,Orn,Ile,Leu,Phe, 0.828; Cit,Arg,Pro,Leu,Phe, 0.828; Val,Orn,Lys,Leu,Phe, 0.828; Cit, Pro, Ile, Leu, Phe, 0.826; Arg,Met, Orn, Leu, Phe, 0.826; Cit,Arg,Orn,Leu,Phe, 0.826; Gln,Arg,Pro,Leu,Phe, 0.824; Ser,Pro,Leu,Phe,Trp, 0.824; Cit,Pro,Tyr,Leu,Phe, 0.822; Ser,Cit,Orn,Leu,Phe, 0.822; Arg,Pro,Val,Leu,Phe, 0.82; His,Arg,Pro,Leu,Phe, 0.82; Ser,Cit,Pro,Leu,Phe, 0.82; Met,Orn,Lys,Leu,Phe, 0.82; Cit,Val,Orn,Leu,Phe, 0.82; His,Val,Orn,Leu,Phe, 0.82; Thr,Pro,Leu,Phe,Trp, 0.819; Arg,Pro,Leu,Phe,Trp, 0.817; Gln,Cit,Leu,Phe,Trp, 0.817; Arg,Pro,Met,Leu,Phe, 0.815; Cit,Arg,Leu,Phe,Trp, 0.815; Cit,Arg,Met,Leu,Phe, 0.815; His,Ala,Orn,Leu,Phe, 0.815; Asn,Pro,Leu,Phe,Trp, 0.815; His,Cit,Lys,Leu,Phe, 0.815; Ser,Tyr,Leu,Phe,Trp, 0.815; Gln,Val,Orn,Phe,Trp, 0.813; Ala,Val,Orn,Phe,Trp, 0.813; Gln,Cit,Lys,Leu,Phe, 0.813; Ala,Pro,Met,Leu,Phe, 0.813; Cit,Met,Leu,Phe,Trp, 0.811; Ser,Cit,Lys,Leu,Phe, 0.811; Gly,His,Cit,Leu,Phe, 0.811; Arg,Ile,Leu,Phe,Trp, 0.809; Glu,Ser,Cit,Leu,Phe, 0.809; Asn,Thr,Cit,Leu,Phe, 0.809; Cit,Val,Lys,Leu,Phe, 0.809; ABA,Tyr,Leu,Phe,Trp, 0.809; Ser,ABA,Leu,Phe,Trp, 0.809; Gln,Lys,Leu,Phe,Trp, 0.807; His,ABA,Leu,Phe,Trp, 0.807; Pro,Tyr,Val,Leu,Phe, 0.807; Ala,Arg,ABA,Leu,Phe, 0.805; Gly,Lys,Leu,Phe,Trp, 0.805; Pro,ABA,Tyr,Leu,Phe, 0.805; Asn,Pro,ABA,Leu,Phe, 0.805; Glu,His,Leu,Phe,Trp, 0.803; Ala,Arg,Val,Orn,Phe, 0.803; Ser,Arg,Val,Orn,Phe, 0.803; Gln,Ala,Met,Leu,Phe, 0.803; Cit,Val,Ile,Leu,Phe, 0.802; Asn,His,Arg,Leu,Phe, 0.802; Thr,Ile,Leu,Phe,Trp, 0.802; Ser,Gly,Orn,Phe,Trp, 0.802; Gln,Pro,Val,Leu,Phe, 0.802; Glu,Pro,Tyr,Leu,Phe, 0.802; Tyr,Ile,Leu,Phe,Trp, 0.8; Gln,Pro,Ile,Leu,Phe, 0.8; Glu,Asn,Val,Orn,Phe, 0.8; Arg,Pro,Val,Ile,Phe, 0.798; Ser,ABA,Orn,Phe,Trp, 0.798; Glu,Arg, Ile,Leu, Phe, 0.796; His, Pro,Val,Orn, Phe, 0.796; Tyr,Met, Ile,Leu, Phe, 0.796; Gly,ABA,Val,Orn, Phe, 0.796; Ser,Ala,Orn,Phe,Trp, 0.794; Ser,Met,Ile,Leu,Phe, 0.794; His,Arg,Tyr,Leu,Phe, 0.792; Ser,Cit,Orn,Phe,Trp, 0.792; Ser,Asn,Orn,Phe,Trp, 0.792; Cit,Pro,Val,Ile,Phe, 0.79; Thr,Tyr,Val,Orn,Phe, 0.79; Arg,Tyr,Orn,Phe,Trp, 0.786; His,Arg,Val,Lys,Phe, 0.786; Gly,Tyr,Val,Orn,Phe, 0.786; Tyr,Val,Ile,Leu,Phe, 0.786; Asn,His,Cit,Val,Phe, 0.784; Glu,Arg,Met,Leu,Phe, 0.783; Orn,Lys,Ile,Phe,Trp, 0.783; Glu,Arg,Tyr,Leu,Phe, 0.781; Ser,Gln,Cit,Val,Phe, 0.779; Gly,Gln,Arg,Val,Phe, 0.777; Asn,Gln,Cit,Val,Phe, 0.777

[115. Formulae with six explanatory variables obtained from amino acids after the start of treatment]

Arg,Orn,Lys,Leu,Phe,Trp, 0.854; Thr,Orn,Lys,Leu,Phe,Trp, 0.853; Ser,Val,Orn,Leu,Phe,Trp, 0.847; Ala, Pro, Orn, Leu, Phe, Trp, 0.845; Pro,ABA, Orn, Ile, Leu, Phe, 0.843; Ala,Pro,Tyr,Orn,Leu,Phe, 0.843; Thr,Ala,Pro,Orn,Leu,Phe, 0.843; Pro,Tyr,Orn,Leu,Phe,Trp, 0.841; Ala,Pro,Val,Orn,Leu,Phe, 0.841; Glu,Met,Orn,Ile,Leu,Phe, 0.837; Glu,Cit,Arg,Pro,Leu,Phe, 0.836; Cit,Arg,Pro,Orn,Leu,Phe, 0.836; Glu,Thr,Pro,Orn,Leu,Phe, 0.836; Gln,Orn,Ile,Leu,Phe,Trp, 0.834; Ser,Ala,Orn,Leu,Phe,Trp, 0.834; Thr,Ala,Orn,Ile,Leu,Phe, 0.834; Gln,Arg,Pro,Orn,Leu,Phe, 0.832; Cit,Arg,Pro,Ile,Leu,Phe, 0.832; Ala,Arg,Pro,Orn,Leu,Phe, 0.832; Glu,Gly,Cit,Pro,Leu,Phe, 0.832; Gln,Pro,Orn,Ile,Leu,Phe, 0.832; Thr,Cit,Arg,Orn,Leu,Phe, 0.832; Asn,Gly,Arg,Orn,Leu,Phe, 0.832; Glu,Pro,ABA,Orn,Leu,Phe, 0.832; His,Orn,Ile,Leu,Phe,Trp, 0.83; Arg,Pro,Orn,Leu,Phe,Trp, 0.83; Cit,Arg,Pro,Met,Leu,Phe, 0.83; Cit,Pro,ABA,Orn,Leu,Phe, 0.83; Asn,Arg,ABA,Orn,Leu,Phe, 0.83; Gly,Arg, Pro, Orn, Leu, Phe, 0.828; Gly,Cit,Arg,Pro,Leu,Phe, 0.828; Asn,Cit,Arg,Pro,Leu,Phe, 0.828; Cit,Arg,Pro,Leu,Phe,Trp, 0.828; Glu,Asn,Cit,Pro,Leu,Phe, 0.828; Arg,Pro,ABA,Orn,Leu,Phe, 0.826; His,Arg,Pro,Orn,Leu,Phe, 0.826; Cit,Arg,Pro,Tyr,Leu,Phe, 0.826; Glu,Cit,Pro,Ile,Leu,Phe, 0.826; Gln,Arg,Pro,Leu,Phe,Trp, 0.826; Cit,Pro,ABA,Met,Leu,Phe, 0.826; Asn,Cit,Pro,Ile,Leu,Phe, 0.826; Thr,Ala,Cit,Pro,Leu,Phe, 0.826; Cit,Pro,Orn,Lys,Leu,Phe, 0.826; Glu,Cit,Arg,Orn,Leu,Phe, 0.826; Glu,Arg,Pro,Orn,Leu,Phe, 0.824; Cit,Arg,Pro,Lys,Leu,Phe, 0.824; Ser,Cit,Pro,Met,Leu,Phe, 0.824; Glu,Ser,Cit,Pro,Leu,Phe, 0.824; Asn,Cit,Arg,Ile,Leu,Phe, 0.824; Glu,Arg,ABA,Orn,Leu,Phe, 0.824; Ala,Cit,Pro,Leu,Phe,Trp, 0.822; Glu,His,Cit,Pro,Leu,Phe, 0.822; Thr,Cit,Pro,Val,Leu,Phe, 0.822; Ser,Thr,Cit,Pro,Leu,Phe, 0.822; Thr,Arg,Val,Orn,Leu,Phe, 0.822; Ser,His,Arg,Orn,Leu,Phe, 0.822; Glu,Cit,Pro,Leu,Phe,Trp, 0.82; Gln,Arg,Pro,Ile,Leu,Phe, 0.82; Ala,Arg,Orn,Lys,Leu,Phe, 0.82; Arg,Pro,ABA,Tyr,Leu,Phe, 0.82; Glu,Gly,Thr,Arg,Leu,Phe, 0.82; Glu,Gln,Arg,Pro,Leu,Phe, 0.819; Glu,Cit,Arg,Ile,Leu,Phe, 0.819; Glu,Cit,Arg,Leu,Phe,Trp, 0.819; Ala,Arg,Pro,Leu,Phe,Trp, 0.817; Ser,Arg,Tyr,Orn,Leu,Phe, 0.817; Glu,Cit,Arg,ABA,Leu,Phe, 0.817; His,Arg,Pro,Lys,Leu,Phe, 0.815; Arg,Pro,Met,Lys,Leu,Phe, 0.815; Arg,Pro,Met,Ile,Leu,Phe, 0.815; Gly,Cit,Pro,Leu,Phe,Trp, 0.815; Glu,Cit,Pro,Val,Leu,Phe, 0.815; Thr,Arg,Tyr,Lys,Leu,Phe, 0.815; Glu,Arg,Pro,ABA,Leu,Phe, 0.813; Gly,Cit,Met,Ile,Leu,Phe, 0.813; Glu,Ala,Cit,Arg,Leu,Phe, 0.813; His,Cit,Pro,Val,Phe,Trp, 0.811; Glu,Cit,ABA,Ile,Leu,Phe, 0.809; Gln,Cit,Lys,Ile,Leu,Phe, 0.809; His,Ala,Cit,Leu,Phe,Trp, 0.809; Glu,Arg,Pro,Met,Leu,Phe, 0.805; Gly,His,Arg,Ile,Leu,Phe, 0.805; Glu,Arg,Pro,Leu,Phe,Trp, 0.803; Glu,Thr,Cit,Arg,Leu,Phe, 0.803; Glu,His,Cit,Arg,Leu,Phe, 0.803; Gly,Cit,Lys,Ile,Leu,Phe, 0.803; Gln,Arg,Pro,Val,Ile,Phe, 0.802; Gly,His,Arg,Tyr,Leu,Phe, 0.802; Gln,Arg,ABA,Ile,Leu,Phe, 0.8; Asn,Ala, Cit, Pro,Val, Phe, 0.8; Glu, Cit,Arg,Met, Leu, Phe, 0.798; Gly,Cit,Pro,Val,Phe,Trp, 0.796; Asn,Ala,Orn,Lys,Phe,Trp, 0.796; Ser,Arg,Val,Lys,Leu,Phe, 0.796; His,Arg,ABA,Ile,Leu,Phe, 0.792; Glu,Ala,Arg,Ile,Leu,Phe, 0.792; Glu,Cit,Arg,Pro,Val,Phe, 0.79; Gly, Cit, Pro,Val, Orn, Phe, 0.79; Thr,Cit,Pro,Val,Ile,Phe, 0.79; Cit,Pro,ABA,Val,Ile,Phe, 0.788

[116. Formulae with two explanatory variables obtained from amino acids and amino acid-related metabolites after the start of treatment]

Trp,Kyn, 0.8; Val,Kyn, 0.788; Leu,hKyn, 0.788; Leu,NP, 0.788; AnthA,NP, 0.786; Val,NP, 0.786; Cit,NP, 0.784; Leu,Kyn, 0.784; Leu,AnthA, 0.784; Orn,Trp, 0.783; Orn,NP, 0.781; Val,hKyn, 0.781; Ile,Kyn, 0.781; Cit,QA, 0.779; AnthA,QA, 0.777; Leu,QA, 0.777; Thr,Kyn, 0.777; ABA,QA, 0.777; hKyn,XA, 0.777; Met,Kyn, 0.773; Arg,NP, 0.773; Thr,NP, 0.773; Val,QA, 0.771; Ile,AnthA, 0.769; Cit,Kyn, 0.769; Val,AnthA, 0.767; QA,NP, 0.766; QA,XA, 0.764; Ile,QA, 0.762; XA,NP, 0.762; Met,QA, 0.76; hKyn,NP, 0.758; Ile,NP, 0.756; His,Kyn, 0.756; Trp,QA, 0.754; Kyn,NP, 0.754; Orn,QA, 0.754; Thr,AnthA, 0.752; Trp,NP, 0.752; ABA,NP, 0.752; Thr,QA, 0.75; Trp,hKyn, 0.75; Asn,NP, 0.75; Arg,Kyn, 0.749; Val,Orn, 0.749; Cit,hKyn, 0.749; Ser,Val, 0.749; Gln,NP, 0.747; ABA,Kyn, 0.747; His,hKyn, 0.745; Tyr,AnthA, 0.743; Asn,QA, 0.743; Ser,NP, 0.743; Ala,NP, 0.743; Tyr,NP, 0.743; Lys,NP, 0.743; Kyn,XA, 0.743; Asn,hKyn, 0.743; Trp,AnthA, 0.741; hKyn,AnthA, 0.741; Cit,Val, 0.741; Val,Trp, 0.741; Tyr,Val, 0.741; Arg,QA, 0.739; Tyr,QA, 0.739; Lys,QA, 0.739; Met,NP, 0.739; His,QA, 0.737; Arg,Val, 0.737; Cit,Trp, 0.737; Ile,hKyn, 0.737; Gln,Val, 0.737; Orn,Leu, 0.737; His,NP, 0.735; Val,XA, 0.732; Arg,Leu, 0.73; Thr,hKyn, 0.73; Arg,Trp, 0.73; Val,Ile, 0.73; Met,hKyn, 0.728; Thr,Cit, 0.726; Leu,Trp, 0.726; Kyn,QA, 0.726; Cit,Leu, 0.724; hKyn,QA, 0.722; Ser,QA, 0.72; Arg,hKyn, 0.718; Arg,Orn, 0.718; Arg,AnthA, 0.715; Ala,QA, 0.715; Thr,Val, 0.715; His,Val, 0.715; Thr,Trp, 0.713; Val,Leu, 0.713; Gln,QA, 0.711; Thr,Orn, 0.711; Asn,Arg, 0.711; Thr,Arg, 0.709; AnthA,Kyn, 0.709; Arg,Ile, 0.701

[117. Formulae with three explanatory variables obtained from amino acids and amino acid-related metabolites after the start of treatment]

ABA,Orn,QA, 0.83; Orn,Trp,Kyn, 0.82; Orn,Trp,QA, 0.813; Cit,Val,Kyn, 0.813; Leu,AnthA,Kyn, 0.809; Cit,Leu,QA, 0.807; Cit,Tyr,QA, 0.807; Orn,Trp,NP, 0.807; Cit,ABA,QA, 0.805; Val,hKyn,AnthA, 0.805; Orn,Leu,hKyn, 0.805; Ile,AnthA,Kyn, 0.803; Ile,Trp,Kyn, 0.803; Trp,AnthA,Kyn, 0.802; Val,Orn,Kyn, 0.802; Leu,Kyn,NP, 0.802; Cit,AnthA,NP, 0.802; Leu,AnthA,NP, 0.802; Val,AnthA,Kyn, 0.8; Val,Trp,Kyn, 0.8; Cit,Ile,QA, 0.8; Trp,Kyn,XA, 0.8; Cit,hKyn,NP, 0.8; Cit,Val,NP, 0.8; Cit,Tyr,Kyn, 0.8; Cit,Met,QA, 0.798; Thr,AnthA,Kyn, 0.798; Val,Kyn,XA, 0.798; ABA,Trp,Kyn, 0.798; Arg,Val,Kyn, 0.796; Ser,Val,Kyn, 0.796; Ser,Trp,Kyn, 0.796; Asn,Trp,Kyn, 0.796; Thr,Leu,Kyn, 0.796; hKyn,AnthA,XA, 0.794; Cit,Leu,NP, 0.794; Orn,Ile,QA, 0.794; Thr,Val,Kyn, 0.792; Met,Trp,Kyn, 0.792; hKyn,AnthA,NP, 0.792; Thr,Trp,Kyn, 0.792; Orn,Leu,Trp, 0.792; Asn,Val,Kyn, 0.79; Tyr,Val,Kyn, 0.79; Orn,Lys,Trp, 0.79; His,Ile,Kyn, 0.79; Val,Met,Kyn, 0.788; Val,Kyn,QA, 0.788; ABA,AnthA,QA, 0.788; Kyn,XA,NP, 0.788; Tyr,Orn,Trp, 0.788; Tyr,AnthA,QA, 0.786; Arg,Trp,Kyn, 0.786; Ile,AnthA,QA, 0.786; Orn,XA,NP, 0.786; Cit,Trp,hKyn, 0.786; Val,Ile,Kyn, 0.784; Tyr,AnthA,Kyn, 0.784; Val,Kyn,NP, 0.783; Arg,Orn,Trp, 0.783; Thr,Cit,QA, 0.781; Cit,Arg,QA, 0.781; Met,Kyn,NP, 0.781; Val,Leu,Kyn, 0.779; Orn,QA,NP, 0.779; Cit,Lys,QA, 0.779; His,Cit,QA, 0.779; Val,AnthA,XA, 0.777; Val,Ile,AnthA, 0.777; Arg,AnthA,NP, 0.777; Val,Orn,hKyn, 0.775; AnthA,QA,NP, 0.775; Cit,QA,XA, 0.775; Thr,Arg,Kyn, 0.775; Ala,Val,AnthA, 0.775; AnthA,Kyn,NP, 0.773; Met,AnthA,QA, 0.773; Val,Met,AnthA, 0.773; Arg,Kyn,XA, 0.773; Arg,QA,XA, 0.773; Arg,Met,Kyn, 0.773; Thr,AnthA,QA, 0.771; Val,Orn,AnthA, 0.771; Val,Orn,Trp, 0.771; Arg,Val,AnthA, 0.769; Thr,hKyn,AnthA, 0.769; Cit,Val,AnthA, 0.769; Thr,Val,AnthA, 0.769; Arg,Trp,QA, 0.769; His,Orn,QA, 0.766; Arg,Kyn,NP, 0.764; ABA,Val,AnthA, 0.764; Ala,AnthA,QA, 0.764; Trp,AnthA,QA, 0.762; His,AnthA,QA, 0.76; Asn,Arg,Kyn, 0.76; Thr,Orn,QA, 0.758; Arg,AnthA,Kyn, 0.758; Arg,Orn,QA, 0.75; Thr,Cit,AnthA, 0.75

[118. Formulae with four explanatory variables obtained from amino acids and amino acid-related metabolites after the start of treatment]

Cit,Leu,hKyn,Kyn, 0.834; Cit,ABA,Ile,Kyn, 0.834; Asn,ABA,Orn,QA, 0.832; ABA,Orn,Trp,Kyn, 0.83; ABA,Orn,Trp,QA, 0.828; ABA,Tyr,Orn,QA, 0.828; Ala,ABA,Orn,QA, 0.826; Gln,Cit,Trp,Kyn, 0.824; Cit,Tyr,Val,Kyn, 0.824; Ala,Orn,Trp,Kyn, 0.822; Orn,Lys,Trp,Kyn, 0.822; Ser,Orn,Trp,QA, 0.82; Cit,Leu,hKyn,QA, 0.819; Val,Orn,AnthA,QA, 0.817; Tyr,AnthA,Kyn,XA, 0.817; His,Orn,Trp,hKyn, 0.817; Tyr,Leu,AnthA,Kyn, 0.817; Asn,Orn,Leu,hKyn, 0.817; Tyr,Orn,Trp,QA, 0.815; Thr,Orn,Trp,Kyn, 0.815; Cit,Tyr,QA,NP, 0.815; Gln,ABA,Orn,QA, 0.815; His,Cit,Trp,QA, 0.815; Lys,Leu,AnthA,QA, 0.815; Orn,Leu,hKyn,XA, 0.815; Val,Lys,AnthA,Kyn, 0.813; Cit,ABA,Lys,QA, 0.813; Thr,Cit,Val,Kyn, 0.813; Cit,ABA,Val,QA, 0.813; Asn,Orn,Trp,hKyn, 0.813; Arg,Orn,Trp,hKyn, 0.813; Thr,Orn,Trp,hKyn, 0.813; Cit,Lys,Leu,QA, 0.813; ABA,Leu,AnthA,Kyn, 0.813; Met,Orn,Kyn,NP, 0.813; Orn,Leu,QA,XA, 0.813; Val,hKyn,AnthA,Kyn, 0.811; Orn,Trp,hKyn,AnthA, 0.811; Ala,Cit,Trp,Kyn, 0.811; Ala,Orn,Trp,hKyn, 0.811; Asn,Val,Orn,Kyn, 0.811; Orn,Trp,AnthA,QA, 0.809; Cit,Tyr,AnthA,QA, 0.809; Val,Orn,hKyn,AnthA, 0.809; Leu,hKyn,AnthA,NP, 0.809; His,Cit,Leu,QA, 0.809; Val,Orn,QA,XA, 0.809; Cit,Tyr,Ile,QA, 0.809; Thr,ABA,Orn,QA, 0.809; Val,AnthA,Kyn,NP, 0.807; Val,Orn,AnthA,NP, 0.807; Cit,Val,Trp,QA, 0.807; Val,Orn,Lys,QA, 0.807; Cit,Leu,AnthA,NP, 0.807; ABA,Val,Orn,QA, 0.805; Orn,hKyn,AnthA,NP, 0.805; Cit,ABA,Ile,QA, 0.803; Ser,Trp,AnthA,Kyn, 0.803; Val,Orn,Ile,QA, 0.803; ABA,Leu,hKyn,AnthA, 0.803; Val,AnthA,Kyn,XA, 0.802; Val,Met,AnthA,Kyn, 0.802; Cit,Val,hKyn,AnthA, 0.802; Arg,Leu,AnthA,Kyn, 0.8; Gln,Val,AnthA,Kyn, 0.8; Val,Ile,AnthA,Kyn, 0.8; Met,Orn,Trp,hKyn, 0.8; Ser,Cit,Val,QA, 0.8; Trp,AnthA,Kyn,XA, 0.8; Tyr,Orn,QA,XA, 0.8; Cit,QA,XA,NP, 0.8; Asn,Arg,Leu,Kyn, 0.798; Val,AnthA,QA,XA, 0.798; Gln,Cit,Tyr,QA, 0.798; Arg,Val,AnthA,Kyn, 0.796; Arg,Ile,AnthA,Kyn, 0.796; ABA,Val,hKyn,AnthA, 0.796; Val,Trp,AnthA,QA, 0.796; Asn,Cit,Val,hKyn, 0.796; Ser,Cit,Trp,QA, 0.796; Tyr,Val,Orn,hKyn, 0.796; Arg,Val,hKyn,AnthA, 0.794; Arg,Val,Kyn,XA, 0.794; Arg,Ile,AnthA,QA, 0.792; Arg,ABA,Leu,Kyn, 0.79; Arg,Val,Trp,Kyn, 0.79; Gln,Arg,Val,Kyn, 0.79; Asn,Leu,hKyn,AnthA, 0.79; Val,Ile,hKyn,AnthA, 0.786; Arg,Tyr,AnthA,Kyn, 0.786; Thr,Cit,Kyn,QA, 0.786; Ser,Val,Orn,hKyn, 0.786; Arg,Val,Lys,Kyn, 0.784; Thr,Tyr,AnthA,QA, 0.784; Arg,Val,AnthA,QA, 0.783; His,Val,Kyn,NP, 0.783; Thr,Arg,Leu,Kyn, 0.781; Gln,Arg,Leu,Kyn, 0.781; Thr,Arg,AnthA,QA, 0.775; Tyr,Trp,AnthA,QA, 0.769

[119. Formulae with five explanatory variables obtained from amino acids and amino acid-related metabolites after the start of treatment]

Cit,Lys,Leu,hKyn,AnthA, 0.845; Cit,ABA,Orn,Ile,QA, 0.834; Cit,Tyr,Lys,Leu,QA, 0.834; Ser,Orn,Lys,Trp,Kyn, 0.834; Ala,Val,Lys,AnthA,Kyn, 0.832; Thr,Cit,ABA,Kyn,QA, 0.832; Ser,Tyr,Orn,Trp,Kyn, 0.832; His,Cit,Val,Kyn,NP, 0.83; Cit,ABA,Kyn,QA,NP, 0.83; Gln,Cit,Trp,hKyn,Kyn, 0.83; His,Cit,Val,Trp,Kyn, 0.828; Ser,Met,Orn,Trp,Kyn, 0.828; Val,Lys,hKyn,AnthA,NP, 0.826; Ala,Cit,ABA,Trp,Kyn, 0.826; Asn,Cit,Trp,Kyn,XA, 0.826; Cit,Arg,Leu,Kyn,NP, 0.824; Gln,Orn,Trp,Kyn,QA, 0.824; Asn,Orn,Trp,Kyn,QA, 0.824; Cit,Tyr,Ile,Trp,Kyn, 0.824; Val,Met,Lys,AnthA,Kyn, 0.822; Asn,Gln,Cit,Trp,Kyn, 0.822; Cit,ABA,Val,Met,Kyn, 0.822; Val,Orn,Ile,Trp,Kyn, 0.822; Ala,Cit,Leu,Trp,Kyn, 0.82; Cit,ABA,Val,Trp,Kyn, 0.82; Ser,Met,Orn,Trp,QA, 0.82; Thr,Cit,Leu,Kyn,QA, 0.82; Tyr,Orn,Trp,hKyn,Kyn, 0.82; Leu,AnthA,Kyn,QA,NP, 0.82; Val,Orn,Trp,AnthA,QA, 0.819; Cit,Tyr,Val,AnthA,QA, 0.819; Gln,Cit,Val,AnthA,Kyn, 0.819; Cit,Val,Ile,Kyn,NP, 0.819; Thr,Cit,ABA,Trp,Kyn, 0.819; His,Orn,Ile,Trp,QA, 0.819; Cit,Lys,Leu,Trp,Kyn, 0.819; Asn,Cit,Val,Orn,Kyn, 0.819; Leu,AnthA,Kyn,XA,NP, 0.819; Orn,Leu,AnthA,QA,XA, 0.819; Orn,Lys,Trp,hKyn,AnthA, 0.817; Val,Orn,hKyn,AnthA,NP, 0.817; Cit,Leu,Trp,AnthA,Kyn, 0.817; Cit,Leu,hKyn,AnthA,QA, 0.817; Ser,Val,Lys,AnthA,Kyn, 0.817; Ser,Orn,Trp,QA,XA, 0.817; His,Orn,Trp,QA,XA, 0.817; Ala,Tyr,Orn,Trp,QA, 0.817; Ala,Orn,Ile,Trp,QA, 0.817; Met,Orn,Trp,QA,NP, 0.817; His,Cit,Val,Kyn,QA, 0.817; Arg,Lys,Trp,AnthA,Kyn, 0.817; Gln,Val,Lys,AnthA,Kyn, 0.815; Tyr,Val,hKyn,AnthA,Kyn, 0.815; Orn,Ile,Trp,QA,NP, 0.815; Cit,Orn,Lys,Trp,hKyn, 0.815; Ala,Orn,Trp,AnthA,QA, 0.813; Cit,Tyr,Val,Trp,Kyn, 0.813; Ser,Val,Ile,AnthA,Kyn, 0.813; Gln,Tyr,Orn,Trp,QA, 0.813; Asn,Cit,Trp,Kyn,NP, 0.813; Asn,Thr,Cit,Trp,Kyn, 0.813; His,Ala,Val,AnthA,Kyn, 0.811; Val,Orn,Leu,Kyn,NP, 0.811; Thr,Arg,Orn,Trp,QA, 0.811; Thr,Cit,Trp,Kyn,NP, 0.811; Cit,Val,Met,AnthA,Kyn, 0.809; Arg,Val,Lys,hKyn,AnthA, 0.809; Thr,Cit,Leu,AnthA,QA, 0.809; Ala,ABA,Val,AnthA,Kyn, 0.809; Arg,Val,Lys,AnthA,Kyn, 0.807; Arg,Val,AnthA,Kyn,NP, 0.807; Arg,Orn,Trp,AnthA,QA, 0.807; Gln,Val,AnthA,Kyn,NP, 0.807; Val,Orn,Leu,Trp,QA, 0.807; Arg,ABA,Val,Orn,QA, 0.807; Thr,Cit,Ile,AnthA,Kyn, 0.805; His,Arg,Val,AnthA,Kyn, 0.803; Gln,Arg,Val,AnthA,Kyn, 0.803; Arg,Orn,Leu,hKyn,AnthA, 0.803; Thr,Cit,Trp,Kyn,QA, 0.803; Arg,Val,Lys,Kyn,NP, 0.803; Arg,Val,Lys,AnthA,QA, 0.802; Ser,Arg,Val,AnthA,Kyn, 0.802; Thr,Cit,Ile,Trp,Kyn, 0.802; His,Cit,Arg,Val,Kyn, 0.802; Arg,Lys,Ile,AnthA,Kyn, 0.802; Arg,Val,AnthA,Kyn,QA, 0.8; Arg,Tyr,Lys,AnthA,QA, 0.8; Arg,ABA,Val,Orn,Kyn, 0.8; Arg,ABA,Val,AnthA,Kyn, 0.798; Thr,Arg,Val,AnthA,Kyn, 0.798; Arg,Val,Leu,AnthA,Kyn, 0.798; Asn,Val,Ile,AnthA,Kyn, 0.798; Gln,Val,Ile,AnthA,Kyn, 0.798; Arg,Val,Ile,AnthA,Kyn, 0.796; Arg,Val,AnthA,Kyn,XA, 0.796; Ala,Arg,Val,Lys,Kyn, 0.796; Arg,Val,Orn,Lys,Kyn, 0.796; Thr,Cit,hKyn,AnthA,QA, 0.792; Arg,Val,Lys,AnthA,XA, 0.784;

[120. Formulae with six explanatory variables obtained from amino acids and amino acid-related metabolites after the start of treatment]

Cit,ABA,Tyr,Lys,AnthA,QA, 0.858; Cit,Tyr,Lys,Trp,AnthA,QA, 0.851; Cit,Tyr,Lys,AnthA,QA,XA, 0.849; Ser,Cit,Tyr,Lys,AnthA,QA, 0.847; Cit,Tyr,Lys,AnthA,Kyn,QA, 0.847; Cit,Tyr,Lys,AnthA,QA,NP, 0.845; Gln,Ala,Cit,Lys,Trp,Kyn, 0.845; Met,Orn,Leu,AnthA,Kyn,NP, 0.843; Ser,Cit,ABA,Tyr,Lys,QA, 0.843; Cit,Tyr,Lys,Ile,AnthA,QA, 0.841; Cit,Tyr,Orn,Lys,AnthA,QA, 0.841; Cit,ABA,Lys,Ile,Trp,Kyn, 0.841; Cit,ABA,Met,Lys,Kyn,QA, 0.836; Cit,ABA,Tyr,Kyn,XA,NP, 0.836; Cit,ABA,Ile,Leu,Kyn,QA, 0.834; Gln,Cit,ABA,AnthA,Kyn,QA, 0.834; Gln,Cit,Met,Trp,Kyn,NP, 0.834; Tyr,Val,Orn,Lys,AnthA,QA, 0.832; Gln,His,Cit,Val,Trp,Kyn, 0.832; Gln,Cit,ABA,Val,Kyn,XA, 0.832; Ala,Orn,Lys,Trp,AnthA,Kyn, 0.83; Cit,Tyr,Orn,Lys,Trp,QA, 0.83; Ala,Cit,Leu,AnthA,Kyn,QA, 0.83; Asn,Gln,Orn,Lys,Trp,QA, 0.828; His,Cit,Trp,AnthA,Kyn,NP, 0.828; Cit,Arg,ABA,Tyr,Lys,QA, 0.828; His,Orn,Lys,Leu,QA,NP, 0.828; Gln,Cit,ABA,Tyr,AnthA,QA, 0.826; His,Orn,Trp,AnthA,Kyn,QA, 0.826; Thr,Val,Orn,Ile,Kyn,NP, 0.826; Thr,Orn,Leu,hKyn,AnthA,Kyn, 0.826; His,Cit,ABA,Orn,QA,NP, 0.826; Cit,Tyr,Orn,Trp,Kyn,XA, 0.826; Ser,Tyr,Trp,AnthA,Kyn,XA, 0.826; Arg,ABA,Orn,Lys,QA,XA, 0.824; Arg,Orn,Trp,hKyn,Kyn,QA, 0.824; Ser,Asn,Thr,Orn,Trp,QA, 0.824; Ser,Asn,Tyr,Orn,Trp,QA, 0.824; Ala,Cit,Val,Ile,Kyn,XA, 0.824; Cit,ABA,Tyr,Orn,AnthA,QA, 0.822; ABA,Orn,Trp,hKyn,AnthA,QA, 0.822; Orn,Leu,Trp,Kyn,QA,NP, 0.822; Cit,ABA,Val,Lys,AnthA,QA, 0.82; Cit,ABA,Tyr,Ile,AnthA,QA, 0.82; Cit,Tyr,Met,AnthA,QA,NP, 0.82; Cit,Tyr,Met,AnthA,Kyn,QA, 0.82; Cit,Ile,Trp,AnthA,Kyn,NP, 0.82; Ala,Val,hKyn,AnthA,Kyn,QA, 0.82; Cit,Tyr,Val,Kyn,QA,XA, 0.82; Thr,Orn,Trp,hKyn,Kyn,NP, 0.82; Val,Orn,Leu,hKyn,AnthA,NP, 0.819; Val,Orn,Leu,hKyn,AnthA,QA, 0.819; Cit,Tyr,Met,Trp,AnthA,QA, 0.817; Cit,ABA,Tyr,hKyn,AnthA,QA, 0.817; Gln,Cit,Tyr,Trp,AnthA,QA, 0.817; Ala,Cit,Met,Trp,Kyn,NP, 0.817; Cit,ABA,Leu,AnthA,QA,XA, 0.815; Thr,Cit,Leu,Trp,Kyn,NP, 0.815; His,Val,Ile,hKyn,AnthA,Kyn, 0.815; Gln,ABA,Val,Lys,AnthA,Kyn, 0.815; Cit,Val,Leu,Trp,hKyn,Kyn, 0.815; Arg,ABA,Val,Lys,AnthA,Kyn, 0.813; Asn,Cit,Arg,Leu,AnthA,Kyn, 0.813; Asn,Cit,Leu,hKyn,AnthA,NP, 0.813; Ser,Thr,Val,Trp,AnthA,Kyn, 0.813; Ala,Cit,Orn,Trp,QA,NP, 0.813; Arg,Tyr,Lys,Leu,AnthA,QA, 0.811; Asn,Cit,Val,Lys,Ile,hKyn, 0.811; Cit,ABA,Val,Trp,AnthA,QA, 0.811; Gln,Val,Trp,AnthA,Kyn,NP, 0.811; Thr,ABA,Val,Orn,hKyn,AnthA, 0.811; Ser,Asn,Thr,Val,AnthA,Kyn, 0.811; Asn,His,Cit,Leu,QA,XA, 0.811; Cit,Ile,Leu,Trp,hKyn,AnthA, 0.811; Cit,Ile,Leu,Trp,hKyn,Kyn, 0.809; Ser,Cit,Leu,Trp,hKyn,Kyn, 0.809; Cit,Tyr,Orn,Trp,hKyn,AnthA, 0.809; Ala,Val,Lys,Leu,AnthA,QA, 0.809; Arg,Lys,Leu,Trp,AnthA,Kyn, 0.807; Ser,Arg,Leu,AnthA,Kyn,NP, 0.807; His,Ala,Arg,Leu,AnthA,Kyn, 0.807; Ser,Thr,Orn,Trp,hKyn,AnthA, 0.807; Arg,ABA,Val,Lys,AnthA,NP, 0.805; Arg,Tyr,AnthA,Kyn,XA,NP, 0.805; Gln,Ala,ABA,Val,AnthA,Kyn, 0.805; Cit,Met,Leu,Trp,hKyn,Kyn, 0.805; Asn,Arg,ABA,Val,Lys,Kyn, 0.803; Thr,Cit,Ile,AnthA,Kyn,QA, 0.803; Asn,Orn,Trp,hKyn,AnthA,NP, 0.803; Arg,Orn,Ile,Leu,AnthA,QA, 0.802; Thr,Tyr,Val,AnthA,Kyn,NP, 0.802; Arg,Tyr,Met,Leu,hKyn,AnthA, 0.8; Ser,Arg,Met,Leu,AnthA,Kyn, 0.798; Arg,ABA,Val,Ile,AnthA,Kyn, 0.796; Asn,Arg,Val,Lys,Kyn,XA, 0.794; Ser,Gln,Val,Ile,AnthA,Kyn, 0.794; Thr,Val,Ile,AnthA,Kyn,QA, 0.794; Asn,Ala,Val,Leu,AnthA,Kyn, 0.792; Thr,Arg,ABA,Lys,AnthA,QA, 0.786; Asn,Gln,Arg,Val,Lys,hKyn, 0.779

[201. Formulae with two explanatory variables obtained from amino acids before the start of treatment]

Ala,Arg, 0.825; Asn,Ala, 0.819; His,Ala, 0.819; Ala,Cit, 0.817; Ala,Orn, 0.817; Ala,Ile, 0.817; Ala,Pro, 0.817; Gln,Ala, 0.817; Ala,Val, 0.817; Ala,Lys, 0.817; Glu,Ala, 0.817; Ala,Met, 0.816; Ala,Trp, 0.816; Ala,Phe, 0.816; Ala,Leu, 0.816; Gly,Ala, 0.814; Ala,Tyr, 0.814; Thr,Ala, 0.813; Ala,ABA, 0.813; Ser,Ala, 0.805; Pro,Lys, 0.766; Orn,Trp, 0.751; His,Pro, 0.746; Arg,Pro, 0.745; Pro,Trp, 0.738; Arg,Trp, 0.738; Lys,Trp, 0.734; Glu,Lys, 0.731; Arg,Orn, 0.729; His,Trp, 0.726; Gly,Lys, 0.726; Thr,Pro, 0.726; His,Arg, 0.726; Orn,Lys, 0.723; Asn,Pro, 0.723; Ser,Lys, 0.723; Gln,Lys, 0.721; His,Lys, 0.721; Pro,Val, 0.721; Arg,Tyr, 0.72; Val,Lys, 0.72; His,Tyr, 0.72; Arg,Val, 0.718; ABA,Lys, 0.718; Asn,Trp, 0.717; Ser,Arg, 0.717; Thr,Lys, 0.715; Thr,Trp, 0.714; Glu,Arg, 0.714; Pro,Tyr, 0.712; Pro,Met, 0.712; Glu,Asn, 0.712; Glu,Trp, 0.711; Cit,Lys, 0.711; Lys,Phe, 0.711; Arg,Ile, 0.711; Pro,ABA, 0.711; Gln,Pro, 0.711; Tyr,Lys, 0.709; Gly,Trp, 0.709; Val,Trp, 0.709; Arg,Lys, 0.707; Tyr,Trp, 0.707; Ile,Trp, 0.707; Ser,Trp, 0.707; Lys,Leu, 0.707; Lys,Ile, 0.707; Asn,Lys, 0.706; Met,Trp, 0.704; Gln,Trp, 0.703; Met,Lys, 0.703; Arg,Leu, 0.703; Phe,Trp, 0.701; Arg,ABA, 0.701; Gln,Arg, 0.701; Gly,Arg, 0.701; Cit,Trp, 0.698; Cit,Arg, 0.698; Gly,Pro, 0.698; Arg,Met, 0.698; Arg,Phe, 0.698; Ser,Pro, 0.698; Val,Leu, 0.698; Cit,Tyr, 0.698; ABA,Trp, 0.697; Thr,Arg, 0.697; Pro,Leu, 0.695; Pro,Ile, 0.693; Leu,Trp, 0.692; Glu,Pro, 0.692; Pro,Phe, 0.69; Thr,Tyr, 0.689; Pro,Orn, 0.687; Cit,Pro, 0.681; Gly,Tyr, 0.678; Asn,Tyr, 0.675; Gln,Tyr, 0.675; Tyr,Orn, 0.675; Asn,Val, 0.666; Tyr,Phe, 0.638

[202. Formulae with three explanatory variables obtained from amino acids before the start of treatment]

Ala,Arg,Met, 0.833; Asn,Ala,Arg, 0.828; His,Ala,Arg, 0.828; Ala,Arg,Leu, 0.828; Ala,Arg,Val, 0.827; Glu,Ala,Arg, 0.827; Ala,Arg,Phe, 0.825; Ala,Arg,Lys, 0.825; Ala,Arg,Pro, 0.825; Ala,Cit,Met, 0.824; Asn,Ala,Cit, 0.824; Ala,Arg,Trp, 0.824; Ala,Arg,Ile, 0.824; Gly,Ala,Arg, 0.824; Glu,Asn,Ala, 0.822; Asn,Ala,Phe, 0.82; Ala,Met,Leu, 0.82; Ala,Cit,Arg, 0.82; His,Ala,Pro, 0.82; Gln,His,Ala, 0.82; Ala,ABA,Met, 0.819; His,Ala,Met, 0.819; Asn,Ala,Pro, 0.819; Ala,Orn,Trp, 0.819; Asn,Gln,Ala, 0.819; Asn,Ala,Leu, 0.819; Ala,Arg,ABA, 0.819; Gly,Thr,Ala, 0.819; Thr,Ala,Phe, 0.819; His,Ala,Val, 0.819; Ala,Met,Trp, 0.817; Ala,Cit,Trp, 0.817; Gly,Ala,Cit, 0.817; Ala,Cit,Lys, 0.817; Glu,Ala,Cit, 0.817; Ala,Leu,Trp, 0.817; Ala,Phe,Trp, 0.817; His,Ala,Orn, 0.817; His,Ala,Leu, 0.817; Ala,Pro,Trp, 0.817; Ala,Ile,Trp, 0.817; Glu,Ala,Orn, 0.817; Gln,Ala,Orn, 0.817; Asn,Thr,Ala, 0.816; Glu,Ala,Met, 0.816; Gly,His,Ala, 0.816; Gln,Thr,Ala, 0.816; Thr,Ala,Arg, 0.814; Asn,Ala,ABA, 0.814; His,Ala,Cit, 0.814; Ala,Val,Met, 0.814; Ala,Pro,Met, 0.814; Ala,Cit,Pro, 0.814; Thr,Ala,Lys, 0.814; Ala,Tyr,Phe, 0.814; Ala,Orn,Phe, 0.814; Gly,Gln,Ala, 0.814; Ala,Val,Orn, 0.814; Ala,Val,Ile, 0.814; Asn,Ala,Trp, 0.813; Ala,ABA,Trp, 0.813; Thr,Ala,Orn, 0.813; Glu,Ala,ABA, 0.813; Gly,Ala,Orn, 0.813; Ala,Tyr,Orn, 0.813; Ala,Pro,Orn, 0.813; Ala,Tyr,Leu, 0.813; Ala,Pro,Phe, 0.813; Gln,Ala,Phe, 0.813; Glu,Ala,Phe, 0.813; Ala,Cit,Orn, 0.811; Asn,His,Ala, 0.811; Asn,Ala,Ile, 0.811; Ser,Ala,Pro, 0.811; Ala,Cit,Ile, 0.811; His,Ala,ABA, 0.811; His,Ala,Lys, 0.811; Gly,Ala,ABA, 0.811; Ala,Lys,Trp, 0.811; Ala,ABA,Tyr, 0.811; Ala,ABA,Ile, 0.811; Gly,Ala,Tyr, 0.811; Ser,Ala,Lys, 0.81; Ala,ABA,Leu, 0.81; Thr,Ala,Val, 0.81; Gln,Ala,ABA, 0.81; His,Thr,Ala, 0.808; Ala,ABA,Phe, 0.808; Ala,Ile,Phe, 0.808; Ala,Tyr,Met, 0.807; Thr,Ala,Trp, 0.807; Ser,Ala,Phe, 0.805; Ser,His,Ala, 0.805; Ser,Ala,Leu, 0.805; Ala,Cit,ABA, 0.805; Ser,Ala,Ile, 0.805; Ser,Ala,Arg, 0.803; Ser,Ala,Trp, 0.803; Ser,Ala,Met, 0.803; Glu,Ser,Ala, 0.802

[203. Formulae with four explanatory variables obtained from amino acids before the start of treatment]

Ala,Arg,Met,Leu, 0.834; His,Ala,Arg,Met, 0.833; Gly,Ala,Arg,Met, 0.831; Ala,Arg,Met,Ile, 0.83; Ala,Arg,Met,Lys, 0.83; His,Ala,Arg,Ile, 0.83; Gln,Ala,Arg,Met, 0.828; Ala,Arg,Orn,Lys, 0.828; Asn,Gly,Thr,Ala, 0.828; Ala,Arg,Val,Met, 0.827; Asn,Ala,Arg,Tyr, 0.827; Thr,Ala,Arg,Orn, 0.825; Thr,Ala,Arg,Met, 0.825; Ala,Cit,Arg,Met, 0.825; Asn,Ala,Cit,Phe, 0.825; Ala,Arg,Tyr,Orn, 0.825; Gly,Ala,Arg,Orn, 0.825; Glu,His,Ala,Arg, 0.825; Glu,Ala,Cit,Met, 0.824; Asn,Ala,Cit,Pro, 0.824; Asn,Ala,Cit,Leu, 0.824; Ala,Arg,Orn,Ile, 0.824; Ala,Arg,Tyr,Met, 0.822; Ala,Arg,ABA,Met, 0.822; Ala,Cit,Met,Trp, 0.822; Ala,Arg,Met,Phe, 0.822; Thr,Ala,Cit,Met, 0.822; Ala,Cit,Pro,Met, 0.822; Asn,Gly,Ala,Val, 0.822; Asn,Gly,Ala,Phe, 0.822; Asn,Gly,His,Ala, 0.82; Asn,Ala,ABA,Leu, 0.819; His,Ala,ABA,Met, 0.819; Asn,Ala,Orn,Lys, 0.819; Asn,Ala,Val,Lys, 0.819; His,Ala,Met,Orn, 0.819; Thr,Ala,Arg,Ile, 0.817; Ala,Cit,Orn,Trp, 0.817; Glu,Thr,Ala,Arg, 0.817; Asn,Ala,Met,Leu, 0.817; Gln,His,Ala,Met, 0.817; Gly,Ala,Met,Ile, 0.816; Asn,Ala,ABA,Trp, 0.816; Ser,Ala,Orn,Trp, 0.816; His,Ala,Cit,Trp, 0.816; Glu,Asn,Ala,Met, 0.816; Asn,Ala,Tyr,Orn, 0.816; Ala,Val,Met,Orn, 0.816; Asn,Gln,Thr,Ala, 0.816; Ala,Cit,Met,Ile, 0.814; His,Ala,Met,Trp, 0.814; Ala,Met,Lys,Ile, 0.814; Thr,Ala,Cit,Pro, 0.814; Asn,His,Ala,Trp, 0.813; Ala,Cit,Val,Orn, 0.813; His,Ala,Cit,Ile, 0.813; Asn,Ala,Val,Leu, 0.813; Asn,Ala,Tyr,Ile, 0.813; His,Ala,ABA,Trp, 0.813; Gly,Thr,Ala,Leu, 0.813; Glu,Ala,Pro,Met, 0.813; Ser,Ala,Arg,Met, 0.811; Thr,Ala,Arg,Leu, 0.811; Thr,Ala,Arg,Pro, 0.811; Gln,Ala,Arg,Lys, 0.811; Ser,His,Ala,Arg, 0.81; Asn,Thr,Ala,Trp, 0.81; Ser,Ala,Tyr,Met, 0.81; Ala,Met,Ile,Phe, 0.81; Ser,Ala,Lys,Leu, 0.81; His,Ala,Lys,Trp, 0.81; Ala,Cit,ABA,Val, 0.81; Glu,Thr,Ala,ABA, 0.81; Thr,Ala,ABA,Val, 0.81; Ser,His,Ala,Cit, 0.808; Glu,Ala,Cit,Orn, 0.808; Ala,Tyr,Ile,Leu, 0.808; His,Ala,Pro,Met, 0.807; Asn,His,Ala,Phe, 0.807; Asn,Ala,Pro,Ile, 0.807; Ser,His,Ala,Leu, 0.807; Ser,Ala,Arg,Phe, 0.805; Ala,Pro,Tyr,Met, 0.805; Ser,Asn,Ala,Tyr, 0.805; Glu,Gln,Ala,Cit, 0.805; Ala,Cit,ABA,Lys, 0.805; Ser,His,Ala,Trp, 0.803; Ser,Ala,Cit,Pro, 0.803; Ser,Ala,Pro,Trp, 0.803; Ser,Asn,His,Ala, 0.803; Ser,Ala,Lys,Phe, 0.803; Ser,Ala,ABA,Orn, 0.803; Gln,Ala,Cit,Leu, 0.803; Ser,Ala,Val,Ile, 0.803; Ser,Gln,Ala,Arg, 0.802; His,Thr,Ala,Val, 0.802; Ser,Ala,Arg,Trp, 0.8; Ser,Ala,Ile,Phe, 0.8; Ser,Asn,Ala,Ile, 0.8; Ser,Ala,Cit,Leu, 0.797

[204. Formulae with five explanatory variables obtained from amino acids before the start of treatment]

Glu,Asn,Ala,Arg,Orn, 0.841; Glu,Ala,Arg,Met,Orn, 0.836; Glu,Ala,Arg,Met,Leu, 0.833; Asn,Gly,Thr,Ala,Arg, 0.831; Ala,Arg,Tyr,Met,Ile, 0.828; Thr,Ala,Arg,Met,Orn, 0.828; Asn,Gly,Ala,Met,Trp, 0.828; His,Ala,Arg,Pro,Phe, 0.828; Ala,Arg,Orn,Leu,Phe, 0.828; Asn,Gly,Ala,Pro,Met, 0.827; Ala,Arg,Val,Met,Ile, 0.825; Ala,Arg,ABA,Met,Orn, 0.825; His,Ala,Arg,Met,Orn, 0.825; Asn,Ala,Cit,ABA,Ile, 0.825; Ser,Asn,Ala,Cit,Ile, 0.825; His,Ala,Cit,Arg,Tyr, 0.825; His,Ala,Arg,Met,Leu, 0.824; Asn,His,Ala,Cit,Phe, 0.824; Asn,Ala,Cit,Pro,ABA, 0.824; Ala,Arg,Tyr,Met,Orn, 0.822; Ser,Thr,Ala,Arg,Orn, 0.822; His,Thr,Ala,Arg,Orn, 0.822; Glu,Ala,Arg,ABA,Met, 0.822; Asn,His,Ala,Cit,Trp, 0.822; Ala,Cit,Met,Lys,Leu, 0.822; Asn,Ala,Pro,Orn,Trp, 0.822; Asn,Gly,Thr,Ala,Lys, 0.822; Asn,Gly,Ala,Pro,Lys, 0.822; Asn,Ala,Cit,ABA,Orn, 0.82; Thr,Ala,Arg,Ile,Phe, 0.82; Gly,Ala,Met,Orn,Trp, 0.82; Asn,Ala,Arg,Tyr,Ile, 0.82; Asn,Ala,Tyr,Lys,Phe, 0.82; Ser,Asn,Gly,Ala,Arg, 0.819; Ala,Cit,Met,Leu,Trp, 0.819; Asn,Thr,Ala,Cit,Tyr, 0.819; Asn,Gly,Ala,Tyr,Met, 0.819; Gln,His,Ala,Arg,Trp, 0.819; Ala,Cit,Orn,Ile,Trp, 0.817; Glu,Gln,Ala,Cit,Met, 0.817; Ala,ABA,Val,Ile,Leu, 0.817; His,Ala,Cit,Tyr,Trp, 0.817; Glu,Ser,Ala,Arg,Met, 0.816; Asn,His,Ala,Cit,Met, 0.816; Asn,Gln,Ala,Met,Trp, 0.816; Glu,Asn,Ala,Val,Trp, 0.816; Thr,Ala,Arg,Lys,Phe, 0.816; Ser,Asn,Ala,Lys,Leu, 0.816; Ser,Ala,Arg,Orn,Lys, 0.814; Ser,Asn,Gly,Ala,Ile, 0.814; Thr,Ala,Arg,Val,Trp, 0.814; Asn,Ala,Met,Lys,Trp, 0.814; Gly,Ala,Cit,Orn,Phe, 0.814; Gly,Ala,ABA,Met,Orn, 0.814; His,Ala,Orn,Lys,Trp, 0.814; Ser,Ala,Arg,Met,Orn, 0.813; Ser,Gly,Ala,Arg,Met, 0.813; Asn,Thr,Ala,Lys,Trp, 0.813; Asn,Ala,ABA,Met,Leu, 0.813; Asn,His,Ala,Val,Phe, 0.813; Ser,Asn,His,Ala,Trp, 0.811; Ala,ABA,Met,Leu,Trp, 0.811; Thr,Ala,Arg,Pro,Lys, 0.811; Asn,Ala,ABA,Val,Ile, 0.811; Glu,Ala,Cit,Orn,Lys, 0.811; Ala,ABA,Ile,Leu,Phe, 0.811; Gln,His,Ala,Orn,Trp, 0.811; Gly,Ala,Tyr,Met,Ile, 0.81; Glu,Ser,Gly,Ala,Cit, 0.81; Ala,Tyr,Ile,Leu,Trp, 0.81; Glu,Ser,Ala,Orn,Trp, 0.81; His,Ala,Pro,Met,Lys, 0.81; Ser,Asn,Gly,Ala,Phe, 0.808; Asn,Thr,Ala,Pro,Trp, 0.808; Ser,Gly,Ala,Val,Lys, 0.808; Ser,Thr,Ala,Cit,Val, 0.808; Ser,Ala,Tyr,Met,Orn, 0.808; Gln,Thr,Ala,Met,Ile, 0.808; Ser,Ala,Arg,Ile,Leu, 0.807; Asn,Ala,Val,Ile,Trp, 0.807; Ala,ABA,Tyr,Met,Phe, 0.807; Ser,Ala,Pro,Tyr,Met, 0.807; Asn,Ala,Pro,Met,Ile, 0.807; Ser,Ala,Met,Orn,Trp, 0.805; His,Thr,Ala,Leu,Trp, 0.805; Ser,Gly,Thr,Ala,Leu, 0.805; Ser,Ala,ABA,Met,Orn, 0.805; Asn,His,Ala,Pro,Lys, 0.805; Ser,Ala,Tyr,Leu,Phe, 0.805; Ser,Ala,Cit,Pro,Phe, 0.803; Glu,Ser,Ala,Met,Trp, 0.802; Glu,Ser,Gln,Ala,Arg, 0.802; His,Thr,Ala,Orn,Trp, 0.802; Thr,Ala,Pro,ABA,Phe, 0.802; Ser,Asn,Ala,Pro,Phe, 0.802; Ser,Gln,His,Ala,Trp, 0.8; Ser,Ala,Met,Orn,Ile, 0.8; Thr,Ala,Ile,Leu,Trp, 0.799; Ser,Asn,His,Ala,Pro, 0.799; Ser,Asn,Ala,ABA,Phe, 0.794

[205. Formulae with six explanatory variables obtained from amino acids before the start of treatment]

Asn,Ala,Arg,Orn,Ile,Trp, 0.837; Ala,Arg,Met,Orn,Ile,Trp, 0.836; Glu,Ala,Arg,Met,Orn,Ile, 0.834; Gly,His,Ala,Arg,ABA,Met, 0.834; Asn,Gly,Ala,Arg,Met,Trp, 0.834; Asn,Ala,Arg,ABA,Tyr,Met, 0.833; Glu,Ala,Arg,Val,Met,Orn, 0.833; Gly,Ala,Arg,Tyr,Val,Met, 0.831; Thr,Ala,Arg,ABA,Met,Ile, 0.83; Asn,Ala,Cit,Arg,ABA,Orn, 0.83; Asn,His,Thr,Ala,Arg,Leu, 0.828; Ala,Arg,Pro,ABA,Met,Ile, 0.827; Ser,Asn,Gly,Ala,Cit,Trp, 0.827; Ser,Asn,Ala,Arg,Pro,Orn, 0.827; Glu,Thr,Ala,Arg,ABA,Orn, 0.827; Asn,Ala,Cit,Met,Lys,Trp, 0.827; Asn,Thr,Ala,Arg,Met,Leu, 0.827; Asn,Gln,Thr,Ala,Arg,ABA, 0.827; Asn,Gln,Thr,Ala,Arg,Ile, 0.827; Ser,Asn,Ala,Cit,ABA,Trp, 0.827; His,Ala,Arg,Val,Met,Ile, 0.825; His,Ala,Arg,Met,Ile,Phe, 0.825; Glu,Ala,Arg,Tyr,Met,Trp, 0.825; Asn,Gly,Ala,ABA,Tyr,Trp, 0.825; Gly,Gln,His,Ala,Met,Trp, 0.825; Ser,Asn,Gly,Ala,ABA,Trp, 0.824; His,Ala,Cit,Arg,Met,Phe, 0.824; Asn,Gly,Ala,Pro,ABA,Trp, 0.824; Asn,His,Thr,Ala,Cit,Met, 0.824; Gly,Ala,Cit,ABA,Tyr,Met, 0.822; Ser,Asn,Gly,Ala,Cit,ABA, 0.822; Thr,Ala,Arg,Pro,Orn,Phe, 0.822; Asn,His,Ala,Cit,Arg,Phe, 0.822; Thr,Ala,Arg,Met,Ile,Leu, 0.82; His,Ala,Cit,Arg,Met,Ile, 0.82; Ala,Cit,ABA,Tyr,Met,Leu, 0.82; Gln,Thr,Ala,Arg,Tyr,Met, 0.82; Ser,His,Ala,Cit,Met,Ile, 0.82; Gly,Ala,Cit,ABA,Val,Met, 0.82; Gln,Thr,Ala,Arg,Ile,Phe, 0.82; His,Ala,Cit,Arg,Tyr,Met, 0.819; Glu,His,Thr,Ala,Arg,Ile, 0.819; Thr,Ala,Arg,Pro,Met,Phe, 0.819; Glu,Gly,Ala,Tyr,Met,Trp, 0.819; Glu,Ala,Cit,Tyr,Met,Leu, 0.817; Glu,Thr,Ala,Arg,Tyr,Met, 0.817; Ser,Thr,Ala,Arg,Pro,Met, 0.817; Glu,Thr,Ala,Cit,Met,Trp, 0.817; Ser,Ala,Cit,ABA,Tyr,Met, 0.816; Ala,Cit,Pro,Met,Orn,Trp, 0.816; His,Ala,Cit,Orn,Ile,Trp, 0.816; Ser,Asn,His,Ala,Arg,Trp, 0.816; His,Ala,Cit,Tyr,Orn,Lys, 0.816; Gly,His,Thr,Ala,ABA,Trp, 0.816; Ala,Cit,Tyr,Met,Ile,Leu, 0.814; Ser,His,Ala,Arg,ABA,Met, 0.814; Ser,Ala,Arg,Orn,Leu,Phe, 0.814; Asn,Ala,Met,Ile,Phe,Trp, 0.814; Ser,Asn,Gly,Ala,Tyr,Trp, 0.813; Gln,Ala,Cit,Tyr,Met,Leu, 0.813; Ser,Asn,Gly,Ala,Pro,ABA, 0.813; Ala,Cit,Pro,Met,Orn,Phe, 0.813; Thr,Ala,Cit,Pro,Tyr,Orn, 0.813; Ser,Gly,Ala,Arg,Pro,Met, 0.811; Ala,Cit,Pro,Met,Ile,Leu, 0.811; Glu,Ser,Asn,His,Ala,Arg, 0.811; Ser,Gly,His,Ala,Lys,Trp, 0.811; Ser,Gly,Ala,Met,Lys,Ile, 0.811; Ser,Gly,Gln,Ala,ABA,Trp, 0.811; Ser,Gly,His,Ala,Ile,Trp, 0.811; Thr,Ala,Arg,ABA,Ile,Leu, 0.81; Ala,Cit,Met,Orn,Ile,Phe, 0.81; Ser,Gly,Gln,Ala,Arg,Phe, 0.81; Asn,Gln,Ala,ABA,Met,Trp, 0.81; Ser,Gly,Thr,Ala,Cit,Ile, 0.81; Ser,His,Thr,Ala,Cit,Lys, 0.81; Glu,Ser,Asn,Gly,Ala,Cit, 0.81; His,Ala,Cit,Tyr,Met,Leu, 0.808; Gln,Ala,Cit,ABA,Ile,Leu, 0.808; Thr,Ala,Arg,Pro,ABA,Lys, 0.808; Ser,Gly,Ala,Tyr,Met,Orn, 0.808; Thr,Ala,Arg,ABA,Tyr,Val, 0.808; Ser,Gly,Ala,Arg,Met,Orn, 0.807; Thr,Ala,Arg,Tyr,Ile,Leu, 0.807; Glu,Ser,Thr,Ala,Arg,Leu, 0.807; Glu,Asn,His,Ala,Pro,Trp, 0.807; Asn,Ala,Val,Met,Ile,Leu, 0.807; Glu,Ser,Ala,Arg,ABA,Phe, 0.805; Ser,Gly,Thr,Ala,ABA,Trp, 0.805; Ser,Thr,Ala,Arg,Lys,Trp, 0.805; Ser,His,Ala,Met,Lys,Trp, 0.805; Ser,Gly,Ala,Cit,ABA,Trp, 0.803; Glu,Ser,Ala,Arg,Val,Leu, 0.803; Glu,Ser,Gln,Ala,Met,Trp, 0.802; Ala,Pro,Tyr,Met,Ile,Trp, 0.802; Glu,Ser,Ala,Arg,Tyr,Phe, 0.8; Ser,Asn,Ala,ABA,Leu,Trp, 0.8; Ser,Asn,Ala,ABA,Val,Trp, 0.8; Ser,Ala,Val,Ile,Leu,Phe, 0.8; Ser,His,Ala,Cit,Orn,Leu, 0.793

[206. Formulae with two explanatory variables obtained from amino acids and amino acid-related metabolites before the start of treatment]

Ala,hTrp, 0.83; Ala,Arg, 0.825; Ala,hKyn, 0.824; Ala,Serot, 0.82; Asn,Ala, 0.819; His,Ala, 0.819; Ala,Cit, 0.817; Ala,Ile, 0.817; Ala,Pro, 0.817; Gln,Ala, 0.817; Ala,Val, 0.817; Ala,Lys, 0.817; Ala,Met, 0.816; Ala,NP, 0.816; Ala,Trp, 0.816; Ala,XA, 0.816; Ala,Tyr, 0.814; His,hTrp, 0.786; Pro,hTrp, 0.783; His,hKyn, 0.779; Asn,hTrp, 0.762; Lys,hTrp, 0.759; Pro,hKyn, 0.752; Lys,hKyn, 0.752; Arg,hTrp, 0.748; Lys,Serot, 0.748; His,Pro, 0.746; Arg,Pro, 0.745; Arg,Serot, 0.741; Pro,Trp, 0.738; Arg,Trp, 0.738; Trp,hTrp, 0.737; hKyn,XA, 0.735; Lys,Trp, 0.734; Trp,hKyn, 0.729; Trp,Serot, 0.729; His,Trp, 0.726; Lys,XA, 0.726; Thr,Pro, 0.726; His,Arg, 0.726; Val,hKyn, 0.723; Asn,Pro, 0.723; Met,hTrp, 0.721; Gln,Lys, 0.721; His,Lys, 0.721; Pro,Val, 0.721; Arg,hKyn, 0.72; Pro,NP, 0.72; Arg,Tyr, 0.72; Val,Lys, 0.72; Gln,hTrp, 0.72; His,Tyr, 0.72; Asn,Trp, 0.717; Thr,Lys, 0.715; Thr,Trp, 0.714; Pro,Serot, 0.712; Pro,Tyr, 0.712; Arg,NP, 0.712; Pro,Met, 0.712; Thr,hTrp, 0.711; Arg,XA, 0.711; Arg,Ile, 0.711; Gln,Pro, 0.711; Tyr,Lys, 0.709; Val,Trp, 0.709; Arg,Lys, 0.707; Thr,hKyn, 0.707; Tyr,Trp, 0.707; Lys,Ile, 0.707; Asn,Arg, 0.707; Trp,NP, 0.706; Trp,XA, 0.706; Asn,Lys, 0.706; Ile,hTrp, 0.704; Met,Trp, 0.704; Gln,Trp, 0.703; Met,Lys, 0.703; Tyr,hTrp, 0.701; Gln,Arg, 0.701; Tyr,Serot, 0.7; Cit,Trp, 0.698; Val,hTrp, 0.698; Cit,Arg, 0.698; Arg,Met, 0.698; Tyr,hKyn, 0.697; Ile,hKyn, 0.697; Met,hKyn, 0.697; Thr,Arg, 0.697; Asn,hKyn, 0.695; Pro,XA, 0.695; Lys,NP, 0.693; Pro,Ile, 0.693; Met,Serot, 0.692; Cit,Pro, 0.681; hTrp,XA, 0.675; Asn,Tyr, 0.675; hTrp,Serot, 0.672; hKyn,Serot, 0.67; hTrp,NP, 0.67; Tyr,NP, 0.663

[207. Formulae with three explanatory variables obtained from amino acids and amino acid-related metabolites before the start of treatment]

Ala,hTrp,Serot, 0.854; Gln,Ala,hTrp, 0.841; Ala,Arg,Serot, 0.836; Ala,Pro,hTrp, 0.834; Ala,hTrp,NP, 0.833; Ala,Arg,Met, 0.833; Ala,Arg,hTrp, 0.831; Ala,Lys,hTrp, 0.831; Ala,Tyr,hTrp, 0.831; Ala,Cit,hTrp, 0.83; Ala,Val,hTrp, 0.83; Ala,Lys,hKyn, 0.83; Ala,hKyn,hTrp, 0.828; Ala,Trp,hTrp, 0.828; Asn,Ala,Arg, 0.828; Ala,hTrp,XA, 0.827; Ala,Trp,hKyn, 0.827; Ala,Met,hKyn, 0.827; Thr,Ala,hKyn, 0.827; Ala,Arg,Val, 0.827; His,Ala,hTrp, 0.825; Ala,Met,Serot, 0.825; Ala,Arg,Lys, 0.825; Ala,Arg,XA, 0.825; Ala,Arg,Pro, 0.825; Ala,Cit,hKyn, 0.824; Ala,Cit,Met, 0.824; Asn,Ala,Cit, 0.824; His,Ala,NP, 0.824; Ala,Arg,Ile, 0.824; Ala,Arg,Tyr, 0.824; His,Ala,hKyn, 0.822; Ala,Pro,Serot, 0.822; Gln,Ala,hKyn, 0.822; Ala,Met,hTrp, 0.82; His,Ala,Serot, 0.82; Ala,Tyr,Serot, 0.82; Ala,Lys,Serot, 0.82; Ala,Cit,Arg, 0.82; His,Ala,Pro, 0.82; Gln,His,Ala, 0.82; Ala,Val,Serot, 0.819; His,Ala,Met, 0.819; Gln,Ala,Arg, 0.819; Asn,Gln,Ala, 0.819; His,Ala,Tyr, 0.819; His,Ala,Val, 0.819; His,Ala,XA, 0.819; Thr,Ala,hTrp, 0.817; Asn,Ala,hTrp, 0.817; Thr,Ala,Serot, 0.817; Ala,Ile,Serot, 0.817; Ala,Met,Trp, 0.817; Ala,Met,NP, 0.817; Ala,Arg,NP, 0.817; Asn,Ala,XA, 0.817; Ala,Met,Lys, 0.817; Ala,Cit,Trp, 0.817; Ala,Pro,NP, 0.817; Ala,Cit,Val, 0.817; Ala,Cit,Lys, 0.817; His,Ala,Ile, 0.817; Ala,Ile,Trp, 0.817; Ala,Trp,XA, 0.817; Gln,Ala,Tyr, 0.817; Ala,hKyn,XA, 0.816; Ala,Trp,Serot, 0.816; Asn,Ala,Tyr, 0.816; Gln,Thr,Ala, 0.816; Ala,Tyr,Trp, 0.816; Gln,Ala,Ile, 0.816; Thr,Ala,Arg, 0.814; Asn,Ala,NP, 0.814; His,Ala,Cit, 0.814; Ala,Val,Met, 0.814; Ala,Pro,Met, 0.814; Ala,XA,NP, 0.814; Ala,Cit,Tyr, 0.814; Ala,Cit,Pro, 0.814; Thr,Ala,XA, 0.814; Gln,Ala,Trp, 0.814; Ala,Val,Ile, 0.814; Ala,Pro,Ile, 0.814; Asn,Ala,Trp, 0.813; Ala,Cit,NP, 0.813; Ala,Ile,NP, 0.813; Ala,Trp,NP, 0.813; Ala,Tyr,NP, 0.813; Asn,His,Ala, 0.811; Asn,Ala,Ile, 0.811; Ala,Cit,Ile, 0.811; Ala,Lys,Trp, 0.811; Thr,Ala,NP, 0.81; Thr,Ala,Val, 0.81; Pro,hKyn,XA, 0.808; His,Thr,Ala, 0.808; Ala,Tyr,Met, 0.807; Pro,hKyn,hTrp, 0.802; Thr,Ala,Tyr, 0.8; Thr,Ala,Ile, 0.799

[208. Formulae with four explanatory variables obtained from amino acids and amino acid-related metabolites before the start of treatment]

Ala,Cit,hTrp,Serot, 0.865; Ala,Pro,hTrp,Serot, 0.859; Ala,hKyn,hTrp,Serot, 0.858; Ala,Val,hTrp,Serot, 0.854; Ala,hTrp,Serot,NP, 0.854; Ala,Tyr,hTrp,Serot, 0.854; Thr,Ala,hTrp,Serot, 0.853; Ala,hTrp,Serot,XA, 0.853; Ala,Ile,hTrp,Serot, 0.853; Ala,Trp,hTrp,Serot, 0.853; His,Ala,hTrp,Serot, 0.85; Ala,Met,hTrp,Serot, 0.85; Ala,Lys,hTrp,Serot, 0.85; Gln,Ala,hKyn,hTrp, 0.842; Ala,Cit,Pro,hTrp, 0.842; Ala,Cit,Val,hTrp, 0.841; Asn,Ala,hTrp,Serot, 0.839; Ala,Cit,hTrp,NP, 0.839; Ala,Cit,Lys,hTrp, 0.839; Gln,Ala,hTrp,NP, 0.839; Gln,Ala,Val,hTrp, 0.839; His,Ala,Arg,Serot, 0.839; Ala,Arg,Tyr,Serot, 0.839; Asn,Gln,Ala,hTrp, 0.837; Gln,Ala,Cit,hTrp, 0.837; Ala,Pro,Val,hTrp, 0.837; Ala,Pro,hTrp,NP, 0.837; Ala,Tyr,hKyn,Serot, 0.837; Ala,Arg,Lys,Serot, 0.837; Ala,Arg,Pro,Serot, 0.837; Ala,Arg,Lys,hTrp, 0.836; Ala,Arg,Serot,XA, 0.836; Ala,Pro,Tyr,hTrp, 0.834; Ala,Pro,Ile,hTrp, 0.834; Gln,Ala,Arg,Serot, 0.834; Ala,Val,hTrp,XA, 0.833; Ala,Arg,hKyn,hTrp, 0.833; Ala,Cit,Tyr,hTrp, 0.833; Gln,Thr,Ala,hTrp, 0.831; Ala,Tyr,hTrp,NP, 0.831; Ala,Tyr,Trp,hTrp, 0.831; Ala,Cit,Ile,hTrp, 0.83; Ala,Met,hTrp,NP, 0.83; Ala,Ile,hTrp,NP, 0.83; Ala,Arg,Met,Ile, 0.83; Ala,Ile,hKyn,hTrp, 0.828; His,Ala,Met,hTrp, 0.828; Ala,Lys,hKyn,hTrp, 0.828; Ala,Ile,hTrp,XA, 0.828; Asn,Ala,Cit,Serot, 0.828; His,Ala,Serot,NP, 0.828; Ala,Cit,Lys,Serot, 0.828; His,Ala,Tyr,hTrp, 0.827; His,Ala,Ile,hTrp, 0.827; His,Ala,Lys,hTrp, 0.827; Ala,Arg,hTrp,XA, 0.827; Ala,Val,Met,hTrp, 0.827; Ala,Met,Serot,NP, 0.827; Ala,Lys,Trp,Serot, 0.827; Asn,Ala,hTrp,NP, 0.825; Ala,Met,Lys,hTrp, 0.825; Ala,Met,Trp,hKyn, 0.825; Asn,Ala,hKyn,XA, 0.824; Asn,Ala,Pro,Serot, 0.824; Asn,Ala,Trp,hKyn, 0.824; Thr,Ala,Lys,Serot, 0.824; Thr,Ala,Trp,Serot, 0.822; Asn,Gln,Ala,Serot, 0.822; Asn,Ala,Tyr,hKyn, 0.822; Gln,His,Ala,hKyn, 0.822; Thr,Ala,hKyn,hTrp, 0.82; Thr,Ala,Pro,hTrp, 0.82; Ala,Tyr,Met,hTrp, 0.82; Thr,Ala,Val,hTrp, 0.82; His,Ala,Val,hKyn, 0.82; Ala,Tyr,Met,hKyn, 0.82; Ala,Pro,Ile,hKyn, 0.82; Gln,His,Ala,Serot, 0.82; Asn,His,Ala,hTrp, 0.819; Asn,Ala,Trp,hTrp, 0.819; Thr,Ala,Met,Serot, 0.819; Ala,Val,Met,hKyn, 0.819; Thr,Ala,Met,hTrp, 0.817; Asn,Ala,Lys,hTrp, 0.817; Thr,Ala,Serot,NP, 0.817; Gln,Thr,Ala,Serot, 0.817; Ala,Lys,Ile,hKyn, 0.817; Gln,Ala,Cit,hKyn, 0.816; Gln,Ala,Trp,Serot, 0.816; Ala,Val,Trp,Serot, 0.816; Thr,Ala,Trp,hTrp, 0.814; Gln,Ala,hKyn,XA, 0.814; Thr,Ala,Pro,Serot, 0.814; Ala,Arg,Ile,hKyn, 0.814; Ala,Trp,Serot,NP, 0.814; Ala,Tyr,hKyn,XA, 0.813; Thr,Ala,Ile,Serot, 0.811; Thr,Ala,Tyr,Serot, 0.811; Gln,Ala,Ile,hKyn, 0.811; Thr,Ala,Tyr,hTrp, 0.808

[209. Formulae with five explanatory variables obtained from amino acids and amino acid-related metabolites before the start of treatment]

Ala,Cit,hKyn,hTrp,Serot, 0.87; Gln,Ala,Cit,hTrp,Serot, 0.865; Ala,Cit,Arg,hTrp,Serot, 0.865; Ala,Cit,Tyr,hTrp,Serot, 0.865; Ala,hKyn,hTrp,Serot,XA, 0.864; His,Ala,Pro,hTrp,Serot, 0.864; Ala,Arg,hKyn,hTrp,Serot, 0.864; Ala,Arg,Met,hTrp,Serot, 0.862; Ala,Cit,Val,hTrp,Serot, 0.861; His,Ala,hKyn,hTrp,Serot, 0.859; Ala,Pro,Tyr,hTrp,Serot, 0.859; Ala,Pro,Lys,hTrp,Serot, 0.858; Ala,hTrp,Serot,XA,NP, 0.856; Ala,Val,hTrp,Serot,NP, 0.856; Ala,Tyr,Ile,hTrp,Serot, 0.856; Thr,Ala,Pro,hTrp,Serot, 0.854; His,Ala,hTrp,Serot,XA, 0.854; Thr,Ala,Cit,hTrp,Serot, 0.853; Asn,Ala,Cit,hTrp,Serot, 0.853; His,Ala,Tyr,hTrp,Serot, 0.853; Thr,Ala,Met,hTrp,Serot, 0.851; Thr,Ala,hTrp,Serot,XA, 0.85; Ala,Trp,hTrp,Serot,XA, 0.85; Pro,Val,hKyn,hTrp,XA, 0.85; Ala,Met,hTrp,Serot,XA, 0.848; Thr,Ala,Trp,hTrp,Serot, 0.845; Thr,Ala,Tyr,hTrp,Serot, 0.844; Asn,His,Ala,hTrp,Serot, 0.844; Gln,His,Ala,Arg,hTrp, 0.842; Gln,His,Ala,Tyr,hTrp, 0.842; Gln,Ala,Arg,hKyn,hTrp, 0.842; Ala,Arg,Tyr,Met,Serot, 0.842; Ala,Val,Met,hKyn,Serot, 0.842; Gln,Ala,Ile,hTrp,XA, 0.841; Thr,Ala,Arg,Met,Serot, 0.841; His,Ala,Cit,Met,hTrp, 0.839; Ala,Arg,Tyr,Lys,hTrp, 0.839; Ala,Met,hKyn,Serot,XA, 0.839; Ala,Lys,hKyn,Serot,XA, 0.839; Ala,Cit,Arg,Met,Serot, 0.839; His,Ala,Tyr,hKyn,Serot, 0.839; Asn,Ala,Pro,hKyn,hTrp, 0.837; His,Ala,Tyr,Val,hTrp, 0.837; Gln,Ala,Val,Met,hTrp, 0.837; Ala,Pro,Lys,Ile,hTrp, 0.837; Thr,Ala,Arg,Serot,XA, 0.837; His,Ala,Met,hKyn,Serot, 0.837; Ala,Lys,hTrp,XA,NP, 0.836; Ala,Pro,Trp,hTrp,NP, 0.836; Gln,Ala,Ile,hTrp,NP, 0.836; Thr,Ala,Arg,Tyr,Serot, 0.836; Ala,Val,hKyn,Serot,XA, 0.836; Asn,Ala,Arg,Pro,Serot, 0.836; Gln,Ala,Met,Trp,hTrp, 0.834; Ala,Tyr,Lys,hTrp,NP, 0.834; Thr,Ala,Cit,Arg,Serot, 0.834; Thr,Ala,Cit,hKyn,hTrp, 0.833; Thr,Ala,Cit,hTrp,NP, 0.833; His,Ala,Pro,Tyr,hTrp, 0.833; Gln,Ala,Arg,Met,hTrp, 0.833; Ala,Cit,Trp,hTrp,NP, 0.833; Ala,Tyr,Trp,hKyn,hTrp, 0.833; Ala,Arg,Pro,Ile,hTrp, 0.833; Asn,Ala,Cit,hKyn,Serot, 0.833; Thr,Ala,hKyn,Serot,XA, 0.833; Ala,Ile,hKyn,Serot,XA, 0.833; Ala,Tyr,hKyn,Serot,XA, 0.833; His,Ala,Pro,hTrp,NP, 0.831; Ala,Arg,Met,hTrp,NP, 0.831; His,Ala,Val,Met,hTrp, 0.831; Ala,Lys,Trp,hTrp,NP, 0.831; Ala,Val,hKyn,hTrp,XA, 0.83; His,Ala,Pro,Lys,hTrp, 0.83; Ala,Pro,Val,Ile,hTrp, 0.83; Ala,Tyr,Val,hKyn,hTrp, 0.83; Ala,Pro,Val,Met,hTrp, 0.83; Asn,Ala,Pro,Met,hTrp, 0.828; Ala,Met,Trp,hTrp,NP, 0.828; Ala,Cit,hKyn,Serot,XA, 0.828; Asn,His,Ala,Arg,hTrp, 0.827; Ala,Val,Met,hKyn,hTrp, 0.827; Ala,Lys,Trp,hTrp,XA, 0.827; Ala,Val,Met,Trp,hTrp, 0.827; Thr,Ala,Met,hTrp,NP, 0.825; Asn,Ala,Val,Met,hTrp, 0.825; Asn,His,Ala,Cit,Serot, 0.825; Ala,Tyr,Met,hKyn,XA, 0.825; Thr,Ala,Cit,hTrp,XA, 0.822; Thr,Ala,Val,Met,hTrp, 0.822; Asn,Ala,hTrp,XA,NP, 0.82; Asn,Ala,Ile,Trp,hTrp, 0.82; Ala,Cit,Met,Ile,hKyn, 0.819; Thr,Ala,hTrp,XA,NP, 0.817; Asn,His,Ala,Ile,hTrp, 0.817; Thr,Ala,Pro,Lys,hTrp, 0.816; Asn,Ala,Tyr,hTrp,XA, 0.814; Ala,Pro,Met,Ile,hTrp, 0.813; Asn,His,Ala,Trp,Serot, 0.813; Thr,Ala,Ile,Trp,hTrp, 0.81; Thr,Ala,Tyr,Trp,hTrp, 0.808

[210. Formulae with six explanatory variables obtained from amino acids and amino acid-related metabolites before the start of treatment]

His,Ala,Arg,hTrp,Serot,XA, 0.865; Ala,Cit,Val,hKyn,hTrp,Serot, 0.865; Ala,Cit,Pro,hTrp,Serot,XA, 0.865; Gln,Thr,Ala,Arg,hTrp,Serot, 0.862; Thr,Ala,Cit,hKyn,hTrp,Serot, 0.862; Ala,Arg,Pro,Met,hTrp,Serot, 0.862; Asn,His,Ala,Arg,hTrp,Serot, 0.861; Ala,Cit,Met,Lys,hTrp,Serot, 0.859; Ala,Arg,Tyr,Trp,hTrp,Serot, 0.859; Ala,Pro,Tyr,hTrp,Serot,NP, 0.859; Thr,Ala,Arg,Ile,hTrp,Serot, 0.858; Ala,Cit,Arg,hKyn,hTrp,Serot, 0.858; Gln,Ala,Lys,hKyn,hTrp,Serot, 0.858; Gln,Thr,Ala,Ile,hTrp,Serot, 0.856; Ala,Val,hKyn,hTrp,Serot,XA, 0.856; Ala,Tyr,Val,Trp,hTrp,Serot, 0.856; Thr,Ala,Arg,Pro,hTrp,Serot, 0.854; Ala,Met,Lys,hKyn,hTrp,Serot, 0.854; Thr,Ala,hTrp,Serot,XA,NP, 0.853; Thr,Ala,Trp,hKyn,hTrp,Serot, 0.853; Asn,Ala,Trp,hKyn,hTrp,Serot, 0.853; Ala,Met,Trp,hTrp,Serot,XA, 0.853; Ala,Val,Ile,hTrp,Serot,NP, 0.853; Thr,Ala,Arg,Met,hTrp,Serot, 0.851; Ala,Tyr,Met,hKyn,hTrp,Serot, 0.851; Asn,Gln,Ala,Val,hTrp,Serot, 0.851; His,Ala,Lys,hTrp,Serot,NP, 0.851; His,Ala,Pro,hKyn,hTrp,XA, 0.85; Ala,Met,Lys,Trp,hTrp,Serot, 0.85; Ala,Lys,Trp,hTrp,Serot,NP, 0.85; Thr,Ala,Arg,Val,hTrp,Serot, 0.848; Ala,Arg,Met,Ile,hTrp,Serot, 0.848; Thr,Ala,Ile,hKyn,hTrp,Serot, 0.848; Ala,Pro,Met,Trp,hTrp,Serot, 0.848; His,Thr,Ala,Arg,Ile,Serot, 0.848; Ala,Lys,Ile,Trp,hTrp,Serot, 0.848; Ala,Arg,Met,Ile,hKyn,Serot, 0.848; Gln,Ala,Pro,Val,hTrp,XA, 0.848; Ala,Tyr,Met,hTrp,Serot,NP, 0.847; Ala,Cit,Tyr,Met,hKyn,hTrp, 0.847; His,Thr,Ala,Ile,hTrp,Serot, 0.845; Ala,Cit,Arg,Tyr,Met,hTrp, 0.845; Asn,Ala,Cit,Tyr,hKyn,hTrp, 0.845; Thr,Ala,Ile,hTrp,Serot,XA, 0.844; Asn,Thr,Ala,Arg,Ile,Serot, 0.844; Gln,Ala,Cit,Met,hKyn,hTrp, 0.844; His,Ala,Arg,Pro,Ile,hTrp, 0.844; Ala,Cit,Pro,hTrp,XA,NP, 0.844; Asn,Gln,Ala,Pro,hKyn,hTrp, 0.842; Thr,Ala,Met,Ile,hTrp,Serot, 0.841; Asn,Ala,Lys,hTrp,Serot,XA, 0.841; Asn,Gln,His,Thr,Ala,hTrp, 0.841; Asn,Ala,Cit,Met,hKyn,hTrp, 0.841; Asn,Gln,Ala,Val,hTrp,XA, 0.841; Thr,Ala,Ile,Trp,hTrp,Serot, 0.839; Ala,Arg,Lys,hKyn,hTrp,XA, 0.839; Ala,Cit,Val,Ile,hTrp,XA, 0.839; Ala,Pro,Tyr,Val,hTrp,XA, 0.839; Asn,Thr,Ala,Arg,Val,Serot, 0.839; Thr,Ala,Arg,Tyr,Ile,Serot, 0.839; His,Ala,Cit,Arg,Ile,hTrp, 0.837; Gln,Thr,Ala,Arg,Ile,Serot, 0.837; His,Ala,Met,Lys,hKyn,hTrp, 0.836; Thr,Ala,Arg,Ile,Serot,NP, 0.836; Thr,Ala,Arg,Val,Met,Serot, 0.836; Asn,Ala,Cit,Arg,hTrp,NP, 0.836; Ala,Pro,Val,Ile,Trp,hTrp, 0.836; Ala,Met,Ile,hTrp,Serot,NP, 0.834; Thr,Ala,Cit,Met,hTrp,NP, 0.834; Thr,Ala,Cit,Arg,hTrp,NP, 0.834; Asn,Gln,Ala,Ile,hKyn,hTrp, 0.834; Thr,Ala,Arg,Val,Ile,Serot, 0.834; Asn,His,Ala,Pro,Val,hTrp, 0.834; Asn,His,Ala,Lys,hKyn,Serot, 0.834; His,Thr,Ala,Cit,Lys,hTrp, 0.833; Thr,Ala,Cit,Arg,Met,hTrp, 0.833; Ala,Cit,Trp,hKyn,hTrp,XA, 0.831; His,Ala,Lys,hTrp,XA,NP, 0.831; Ala,Arg,Trp,hKyn,hTrp,XA, 0.83; Thr,Ala,Cit,Arg,Trp,hTrp, 0.828; Ala,Tyr,Val,Ile,hKyn,hTrp, 0.828; Ala,Val,Ile,Trp,hTrp,XA, 0.828; Thr,Ala,Cit,Pro,Lys,hTrp, 0.827; Thr,Ala,Arg,Tyr,Val,hTrp, 0.827; Asn,His,Ala,Pro,hTrp,XA, 0.827; His,Ala,Val,Met,hTrp,XA, 0.825; His,Thr,Ala,Met,hKyn,hTrp, 0.822; Asn,Thr,Ala,Arg,Tyr,hTrp, 0.822; His,Thr,Ala,Tyr,hKyn,hTrp, 0.82; Thr,Ala,Arg,Lys,Trp,hTrp, 0.82; Thr,Ala,Arg,Val,Ile,hTrp, 0.819; Thr,Ala,Arg,Ile,hKyn,hTrp, 0.819; Thr,Ala,Cit,Lys,hTrp,XA, 0.819; His,Thr,Ala,Trp,hTrp,NP, 0.819; Asn,Ala,Val,Ile,Trp,hTrp, 0.816; His,Thr,Ala,Tyr,Trp,hTrp, 0.816; Asn,His,Ala,Met,hTrp,XA, 0.816; Thr,Ala,Ile,Trp,hTrp,NP, 0.811; Thr,Ala,Ile,Trp,hKyn,hTrp, 0.81; Thr,Ala,Val,Met,Ile,hTrp, 0.808

[301. Formulae with two explanatory variables obtained from amino acids before the start of treatment with PFS as evaluation index]

Arg,Met, 0.784; Arg,Phe, 0.756; Arg,Ile, 0.749; Arg,Leu, 0.743; Thr,Arg, 0.733; Ser,Arg, 0.729; Arg,Orn, 0.727; Cit,Arg, 0.727; His,Arg, 0.727; Arg,Pro, 0.727; Arg,Trp, 0.727; Asn,Arg, 0.725; Gln,Arg, 0.719; Ala,Arg, 0.71; Arg,Lys, 0.71; Tyr,Phe, 0.689; His,Ile, 0.684; Phe,Trp, 0.681; Ser,His, 0.681; His,Phe, 0.679; His,Leu, 0.678; His,Val, 0.676; His,Cit, 0.675; His,Met, 0.673; His,Orn, 0.671; His,Tyr, 0.668; His,Pro, 0.665; Met,Trp, 0.662; His,Thr, 0.657; His,Trp, 0.651; Ser,Thr, 0.651; Gln,His, 0.651; Ser,Ala, 0.649; Ser,Trp, 0.648; Ser,Pro, 0.646; Ala,Phe, 0.644; Asn,His, 0.643; Val,Phe, 0.64; Val,Trp, 0.638; Tyr,Trp, 0.637; Thr,Met, 0.637; Gly,His, 0.635; Thr,Ile, 0.633; Thr,Cit, 0.633; Ala,Met, 0.632; Cit,Trp, 0.632; Leu,Trp, 0.629; Thr,Phe, 0.627; Gly,Trp, 0.627; Ile,Trp, 0.627; Thr,Lys, 0.625; Pro,Trp, 0.625; Orn,Trp, 0.625; Gly,Ala, 0.624; Thr,Pro, 0.624; Ser,Lys, 0.622; Asn,Trp, 0.622; Gly,Lys, 0.621; Ala,Cit, 0.621; Gln,Trp, 0.621; Gln,Ala, 0.619; Ala,Leu, 0.619; His,Lys, 0.619; Thr,Tyr, 0.619; Ala,Ile, 0.617; His,Ala, 0.617; Gly,Thr, 0.617; Thr,Leu, 0.617; Thr,Trp, 0.616; Lys,Ile, 0.614; Ala,Val, 0.613; Cit,Lys, 0.611; Gln,Thr, 0.611; Ser,Asn, 0.608; Asn,Thr, 0.608; Ala,Trp, 0.606; Lys,Trp, 0.606; Leu,Phe, 0.606; Ala,Pro, 0.603; Asn,Ala, 0.603; Pro,Phe, 0.602; Pro,Lys, 0.6; Lys,Leu, 0.598; Ala,Tyr, 0.594; Asn,Phe, 0.594; Ala,Lys, 0.592; Gly,Val, 0.584; Val,Lys, 0.583; Asn,Lys, 0.583; Orn,Lys, 0.583; Asn,Gly, 0.581; Gln,Lys, 0.579; Met,Phe, 0.575; Met,Lys, 0.57; Tyr,Lys, 0.567; Gly,Met, 0.557; Gly,Pro, 0.543; Gly,Leu, 0.543; Gly,Ile, 0.54

[302. Formulae with three explanatory variables obtained from amino acids before the start of treatment with PFS as evaluation index]

His,Arg,Met, 0.789; Cit,Arg,Met, 0.786; Arg,Pro,Met, 0.786; Arg,Val,Met, 0.783; Arg,Met,Ile, 0.781; Ala,Arg,Met, 0.776; Ser,Arg,Phe, 0.77; Arg,Met,Leu, 0.768; Gln,Arg,Met, 0.768; Gly,Arg,Met, 0.765; Thr,Arg,Phe, 0.765; His,Arg,Phe, 0.76; Arg,Val,Phe, 0.759; Arg,Ile,Phe, 0.756; Gln,Arg,Phe, 0.756; Ser,Thr,Arg, 0.751; Thr,Arg,Ile, 0.749; Arg,Val,Ile, 0.749; Arg,Ile,Trp, 0.749; Gly,Arg,Ile, 0.748; Ala,Arg,Phe, 0.748; Cit,Arg,Leu, 0.746; Asn,Arg,Phe, 0.746; Arg,Ile,Leu, 0.744; Thr,Arg,Trp, 0.743; Thr,Cit,Arg, 0.743; Gln,Arg,Ile, 0.743; Arg,Lys,Phe, 0.743; Cit,Arg,Val, 0.741; Asn,Arg,Ile, 0.741; Arg,Val,Leu, 0.74; Ser,Arg,Ile, 0.738; Asn,Arg,Val, 0.737; Gln,Arg,Leu, 0.737; Arg,Pro,Ile, 0.735; His,Arg,Ile, 0.735; His,Cit,Arg, 0.732; His,Thr,Arg, 0.732; Arg,Pro,Val, 0.732; Gly,Arg,Val, 0.732; Gly,Arg,Leu, 0.732; Cit,Arg,Pro, 0.73; Asn,Gly,Arg, 0.729; Gly,Cit,Arg, 0.729; His,Arg,Val, 0.729; Asn,Cit,Arg, 0.727; Gly,Gln,Arg, 0.727; Asn,Arg,Orn, 0.725; Thr,Arg,Orn, 0.725; Ala,Arg,Leu, 0.725; Arg,Tyr,Val, 0.724; Cit,Arg,Lys, 0.722; Gln,Cit,Arg, 0.722; Arg,Orn,Trp, 0.721; Ser,Arg,Orn, 0.721; Ser,Gly,Arg, 0.721; Arg,Orn,Leu, 0.721; Gly,Arg,Trp, 0.719; His,Arg,Leu, 0.719; Ser,Arg,Lys, 0.717; His,Arg,Orn, 0.714; Thr,Arg,Tyr, 0.711; Ala,Arg,Trp, 0.706; Gln,Arg,Orn, 0.705; His,Ala,Arg, 0.705; Gln,Arg,Tyr, 0.702; His,Val,Phe, 0.684; His,Arg,Tyr, 0.684; Ile,Phe,Trp, 0.681; Leu,Phe,Trp, 0.679; Arg,Tyr,Trp, 0.679; Ser,Phe,Trp, 0.675; Gly,Phe,Trp, 0.67; Pro,Val,Phe, 0.662; Ala,Phe,Trp, 0.66; Ala,Val,Phe, 0.66; Ala,Orn,Phe, 0.659; Lys,Phe,Trp, 0.657; Ala,Cit,Phe, 0.657; Gln,Phe,Trp, 0.656; Cit,Lys,Phe, 0.654; Gly,Val,Phe, 0.651; Thr,Ala,Phe, 0.651; Ser,Gly,Ala, 0.648; Ala,Pro,Phe, 0.644; Asn,Ala,Phe, 0.644; Ala,Tyr,Trp, 0.64; Thr,Lys,Phe, 0.64; Pro,Lys,Phe, 0.64; Ala,Tyr,Phe, 0.638; Gly,Ala,Phe, 0.635; Gly,Ala,Trp, 0.63; Gly,Ala,Pro, 0.629; Asn,Gly,Ala, 0.627; Val,Lys,Phe, 0.627; Gly,Ala,Orn, 0.625; Gly,Lys,Phe, 0.625; Gly,His,Ala, 0.622; Ser,Ala,Tyr, 0.619; Gln,Ala,Tyr, 0.587

[303. Formulae with four explanatory variables obtained from amino acids before the start of treatment with PFS as evaluation index]

Cit,Arg,Met,Phe, 0.787; His,Arg,Met,Orn, 0.783; Gly,Arg,Met,Trp, 0.783; Asn,Arg,Met,Trp, 0.781; Ala,Arg,Met,Leu, 0.781; His,Arg,Met,Phe, 0.781; Thr,Arg,Met,Phe, 0.775; Thr,Cit,Arg,Phe, 0.771; His,Cit,Arg,Phe, 0.771; Ser,Ala,Arg,Met, 0.771; Arg,Met,Ile,Phe, 0.77; Gln,Arg,Met,Phe, 0.768; Cit,Arg,Ile,Phe, 0.767; Cit,Arg,Tyr,Phe, 0.767; Arg,Met,Lys,Trp, 0.765; Gly,Cit,Arg,Phe, 0.765; Thr,Arg,Ile,Phe, 0.765; Gln,Ala,Arg,Met, 0.765; Ala,Arg,Met,Phe, 0.765; Cit,Arg,Val,Phe, 0.762; His,Thr,Arg,Phe, 0.76; Arg,Val,Ile,Phe, 0.76; Ser,Arg,Met,Lys, 0.759; His,Arg,Ile,Phe, 0.759; Arg,Tyr,Ile,Phe, 0.757; Arg,Pro,Leu,Phe, 0.756; Asn,Arg,Met,Lys, 0.754; Gln,Arg,Val,Phe, 0.754; Ser,Arg,Val,Phe, 0.752; Gly,Arg,Phe,Trp, 0.752; Thr,Ala,Arg,Phe, 0.752; Thr,Arg,Met,Lys, 0.751; His,Arg,Lys,Phe, 0.751; Gln,Ala,Arg,Phe, 0.751; Ala,Arg,Lys,Phe, 0.751; Ala,Arg,Pro,Phe, 0.751; Arg,Ile,Phe,Trp, 0.749; Arg,Val,Lys,Phe, 0.748; Arg,Tyr,Val,Phe, 0.748; Ala,Arg,Val,Phe, 0.748; Arg,Lys,Ile,Phe, 0.746; Gly,Ala,Arg,Phe, 0.746; Ala,Arg,Phe,Trp, 0.746; His,Arg,Tyr,Phe, 0.741; Gly,Arg,Lys,Phe, 0.741; Cit,Arg,Lys,Phe, 0.74; Gly,Cit,Arg,Tyr, 0.738; Ser,Arg,Lys,Phe, 0.738; Arg,Pro,Tyr,Phe, 0.727; Asn,Arg,Tyr,Phe, 0.722; Cit,Arg,Tyr,Trp, 0.717; Ala,Arg,Tyr,Phe, 0.717; His,Ala,Arg,Tyr, 0.706; Ser,Arg,Tyr,Orn, 0.705; Arg,Tyr,Phe,Trp, 0.705; Ala,Arg,Pro,Tyr, 0.7; His,Arg,Tyr,Orn, 0.698; Ala,Arg,Tyr,Ile, 0.698; Ala,Arg,Tyr,Leu, 0.694; Ala,Arg,Tyr,Lys, 0.69; Gly,Ala,Arg,Tyr, 0.69; Asn,Arg,Tyr,Orn, 0.689; Gln,Ala,Arg,Tyr, 0.689; Gln,Arg,Tyr,Orn, 0.687; Arg,Tyr,Ile,Trp, 0.686; Ala,Ile,Leu,Phe, 0.681; Gly,Arg,Tyr,Lys, 0.678; Ala,Cit,Val,Phe, 0.678; Ala,Orn,Leu,Phe, 0.676; Ala,Cit,Leu,Phe, 0.676; Ser,Ala,Leu,Phe, 0.675; Ala,Val,Ile,Phe, 0.675; Ser,Ala,Orn,Phe, 0.671; His,Ala,Orn,Phe, 0.668; Ala,Met,Phe,Trp, 0.662; Gln,Ala,Orn,Phe, 0.662; Ser,Ala,Phe,Trp, 0.662; His,Ala,Cit,Phe, 0.659; Thr,Ala,Met,Phe, 0.657; His,Ala,Met,Phe, 0.656; Ala,Pro,Orn,Phe, 0.656; Ser,Ala,Pro,Phe, 0.654; Thr,Ala,Leu,Phe, 0.654; His,Thr,Ala,Phe, 0.652; Ala,Cit,Lys,Phe, 0.652; Gln,Ala,Phe,Trp, 0.651; Ala,Cit,Tyr,Phe, 0.651; Gly,Thr,Ala,Phe, 0.649; Asn,Gln,Ala,Phe, 0.644; Ser,Ala,Tyr,Phe, 0.644; Ala,Tyr,Leu,Phe, 0.641; Ala,Pro,Tyr,Phe, 0.64; Ala,Pro,Met,Phe, 0.64; Ala,Lys,Ile,Phe, 0.64; Gln,Ala,Tyr,Phe, 0.637; Gln,Ala,Ile,Phe, 0.637; Gly,Ala,Pro,Phe, 0.635; Gly,Ala,Lys,Phe, 0.635; Gly,Ala,Tyr,Phe, 0.624; Ala,Tyr,Ile,Phe, 0.622

[304. Formulae with five explanatory variables obtained from amino acids before the start of treatment with PFS as evaluation index]

His,Cit,Arg,Met,Phe, 0.802; His,Arg,Val,Met,Phe, 0.794; Ala,Arg,Ile,Leu,Phe, 0.783; Ala,Arg,Val,Met,Phe, 0.781; His,Ala,Arg,Met,Ile, 0.779; Thr,Arg,Val,Met,Phe, 0.776; Gly,Ala,Arg,Met,Leu, 0.776; Arg,Tyr,Val,Met,Phe, 0.775; Arg,Met,Ile,Phe,Trp, 0.775; Ser,Ala,Arg,Val,Met, 0.775; Arg,Val,Met,Phe,Trp, 0.775; Thr,Arg,Met,Phe,Trp, 0.773; Ser,Thr,Ala,Arg,Met, 0.773; Ala,Cit,Arg,Met,Phe, 0.773; Gly,Ala,Cit,Arg,Met, 0.771; His,Ala,Arg,Pro,Met, 0.77; Arg,Val,Met,Lys,Phe, 0.77; Ala,Arg,Met,Lys,Phe, 0.77; His,Ala,Arg,Val,Met, 0.768; Ser,Thr,Arg,Phe,Trp, 0.768; Cit,Arg,Val,Ile,Phe, 0.768; Thr,Ala,Arg,Met,Phe, 0.768; Arg,Val,Met,Orn,Phe, 0.767; Ala,Arg,Val,Met,Trp, 0.765; Asn,Ala,Arg,Met,Phe, 0.765; His,Ala,Arg,Val,Phe, 0.763; Asn,Gln,Ala,Arg,Met, 0.762; Arg,Val,Met,Leu,Phe, 0.762; Thr,Ala,Arg,Met,Orn, 0.76; Asn,Arg,Val,Ile,Phe, 0.76; Thr,Ala,Arg,Phe,Trp, 0.759; Ala,Arg,Met,Orn,Phe, 0.759; Asn,His,Ala,Arg,Phe, 0.754; Ala,Cit,Arg,Ile,Phe, 0.752; His,Ala,Arg,Leu,Phe, 0.752; Ala,Arg,Pro,Lys,Phe, 0.752; Gly,Gln,Ala,Arg,Phe, 0.751; His,Ala,Arg,Ile,Phe, 0.751; Ala,Arg,Lys,Phe,Trp, 0.749; Ala,Cit,Arg,Phe,Trp, 0.749; His,Ala,Arg,Lys,Phe, 0.749; Ser,Ala,Cit,Arg,Phe, 0.746; Arg,Orn,Ile,Phe,Trp, 0.743; Gln,Arg,Orn,Phe,Trp, 0.74; His,Arg,Tyr,Val,Phe, 0.74; Ala,Cit,Arg,Val,Phe, 0.74; Arg,Orn,Leu,Phe,Trp, 0.738; Ala,Cit,Arg,Leu,Phe, 0.738; Thr,Arg,Orn,Lys,Phe, 0.735; His,Ala,Arg,Tyr,Met, 0.729; Ala,Arg,Tyr,Met,Phe, 0.727; Arg,Tyr,Orn,Ile,Phe, 0.724; Cit,Arg,Tyr,Lys,Phe, 0.717; Ala,Arg,Tyr,Val,Phe, 0.714; Gly,His,Ala,Arg,Tyr, 0.713; Ala,Arg,Tyr,Leu,Phe, 0.71; Ser,Asn,Ala,Arg,Tyr, 0.706; Arg,Tyr,Orn,Phe,Trp, 0.706; Ala,Arg,Tyr,Orn,Phe, 0.706; Ala,Arg,Tyr,Lys,Trp, 0.703; Asn,Ala,Cit,Arg,Tyr, 0.703; Arg,Pro,Tyr,Orn,Phe, 0.703; Asn,Ala,Arg,Pro,Tyr, 0.7; Asn,Ala,Arg,Tyr,Val, 0.695; Thr,Ala,Arg,Tyr,Lys, 0.694; Ser,Ala,Ile,Leu,Phe, 0.692; Ala,Pro,Ile,Leu,Phe, 0.69; Ala,Met,Ile,Leu,Phe, 0.69; Gln,Ala,Arg,Tyr,Trp, 0.689; Gln,Ala,Cit,Arg,Tyr, 0.686; Arg,Tyr,Orn,Ile,Trp, 0.684; Ala,Cit,Leu,Phe,Trp, 0.679; Gly,Ala,Arg,Tyr,Orn, 0.679; Gln,Ala,Arg,Tyr,Orn, 0.678; Asn,Gly,Ala,Val,Phe, 0.678; Ala,Val,Met,Orn,Phe, 0.676; Thr,Ala,Cit,Leu,Phe, 0.676; Asn,Thr,Ala,Cit,Phe, 0.675; His,Ala,Orn,Leu,Phe, 0.673; Ala,Val,Orn,Leu,Phe, 0.67; Asn,Thr,Ala,Lys,Phe, 0.668; Asn,Ala,Val,Met,Phe, 0.667; Asn,Gln,Ala,Phe,Trp, 0.667; Thr,Ala,Leu,Phe,Trp, 0.663; His,Ala,Val,Leu,Phe, 0.662; Gln,Ala,Val,Leu,Phe, 0.66; His,Ala,Val,Lys,Phe, 0.659; Ala,Cit,Pro,Phe,Trp, 0.659; Ala,Pro,Val,Met,Phe, 0.657; Gln,Thr,Ala,Cit,Phe, 0.657; Gly,Thr,Ala,Cit,Phe, 0.657; Gly,Gln,Ala,Cit,Phe, 0.656; Gln,Ala,Cit,Pro,Phe, 0.656; Ala,Tyr,Val,Ile,Phe, 0.656; Asn,Ala,Pro,Leu,Phe, 0.654; Gly,Ala,Tyr,Val,Phe, 0.652; Asn,Ala,Lys,Leu,Phe, 0.651; Ala,Cit,Tyr,Orn,Phe, 0.648; Ser,Ala,Tyr,Phe,Trp, 0.638; Gln,Ala,Tyr,Leu,Phe, 0.637

[305. Formulae with six explanatory variables obtained from amino acids before the start of treatment with PFS as evaluation index]

His,Ala,Arg,Met,Leu,Phe, 0.803; Ala,Arg,Met,Ile,Leu,Phe, 0.8; Ala,Arg,Met,Lys,Leu,Phe, 0.797; Thr,Ala,Arg,Met,Leu,Phe, 0.795; Ala,Arg,Val,Met,Leu,Phe, 0.794; Ala,Arg,Met,Leu,Phe,Trp, 0.792; Asn,Ala,Arg,Met,Leu,Phe, 0.792; Gly,Ala,Arg,Met,Leu,Phe, 0.792; Ala,Arg,Met,Orn,Leu,Phe, 0.79; Ala,Arg,Pro,Met,Leu,Phe, 0.786; Ala,Arg,Orn,Ile,Leu,Phe, 0.783; Ala,Arg,Val,Ile,Leu,Phe, 0.783; Ala,Arg,Val,Met,Ile,Phe, 0.783; Gly,Ala,Arg,Pro,Met,Leu, 0.781; Ser,Ala,Arg,Ile,Leu,Phe, 0.779; Gly,Thr,Ala,Arg,Met,Leu, 0.776; His,Thr,Ala,Arg,Val,Phe, 0.773; Ser,Ala,Cit,Arg,Val,Met, 0.771; Ser,Thr,Ala,Arg,Val,Phe, 0.771; Gly,Ala,Arg,Pro,Met,Trp, 0.77; Ser,Asn,Ala,Arg,Val,Met, 0.768; His,Arg,Val,Orn,Ile,Phe, 0.768; Ser,Ala,Arg,Met,Ile,Leu, 0.768; Ser,Asn,Ala,Arg,Met,Orn, 0.767; Ala,Cit,Arg,Met,Lys,Phe, 0.767; Asn,Ala,Arg,Met,Lys,Phe, 0.767; Ala,Arg,Tyr,Met,Leu,Phe, 0.767; Asn,Ala,Arg,Met,Orn,Trp, 0.765; His,Thr,Ala,Arg,Pro,Phe, 0.762; Gln,Ala,Arg,Met,Ile,Phe, 0.762; Ala,Arg,Val,Ile,Phe,Trp, 0.76; His,Ala,Arg,Met,Ile,Phe, 0.76; Gln,Ala,Cit,Arg,Lys,Phe, 0.759; Ala,Arg,Pro,Met,Ile,Phe, 0.759; Gly,Ala,Arg,Met,Ile,Phe, 0.759; Gly,Thr,Arg,Val,Orn,Phe, 0.757; His,Ala,Arg,Pro,Lys,Phe, 0.757; Gln,Ala,Arg,Pro,Phe,Trp, 0.756; Asn,Ala,Arg,Val,Lys,Phe, 0.754; Gly,Gln,Ala,Arg,Pro,Phe, 0.752; Gly,Ala,Arg,Pro,Lys,Phe, 0.752; Gln,Ala,Arg,Lys,Leu,Phe, 0.751; Thr,Ala,Cit,Arg,Ile,Phe, 0.751; Gln,Thr,Arg,Val,Orn,Phe, 0.749; Ala,Arg,Tyr,Ile,Leu,Phe, 0.749; Ala,Arg,Val,Met,Lys,Ile, 0.748; Ser,Ala,Cit,Arg,Ile,Phe, 0.748; Ala,Cit,Arg,Pro,Phe,Trp, 0.748; Gly,Ala,Arg,Lys,Leu,Phe, 0.746; His,Ala,Arg,Val,Orn,Phe, 0.744; Ser,Gln,Ala,Arg,Ile,Phe, 0.744; Gly,Ala,Arg,Orn,Leu,Phe, 0.743; Gly,Ala,Arg,Val,Leu,Phe, 0.743; Asn,Gly,Ala,Arg,Orn,Phe, 0.743; Ala,Arg,Tyr,Met,Ile,Phe, 0.738; Ser,Ala,Arg,Pro,Ile,Phe, 0.737; Thr,Ala,Cit,Arg,Orn,Phe, 0.737; Ser,Ala,Arg,Pro,Orn,Phe, 0.735; Arg,Val,Orn,Lys,Leu,Phe, 0.732; His,Ala,Arg,Tyr,Met,Phe, 0.727; Asn,Ala,Arg,Tyr,Met,Leu, 0.725; Gly,Ala,Arg,Tyr,Met,Trp, 0.725; Ala,Arg,Pro,Tyr,Val,Met, 0.724; Gln,Ala,Arg,Tyr,Met,Orn, 0.722; Gly,Ala,Cit,Ile,Leu,Phe, 0.722; Thr,Ala,Arg,Tyr,Leu,Phe, 0.722; Ala,Cit,Arg,Pro,Tyr,Phe, 0.719; His,Ala,Cit,Arg,Tyr,Trp, 0.717; Ala,Arg,Pro,Tyr,Val,Phe, 0.717; Ser,Gly,Ala,Arg,Tyr,Phe, 0.716; Ala,Cit,Arg,Tyr,Leu,Phe, 0.716; Ala,Cit,Arg,Tyr,Ile,Leu, 0.713; Asn,Ala,Arg,Tyr,Orn,Phe, 0.713; Asn,Ala,Arg,Tyr,Phe,Trp, 0.711; Asn,Gly,Ala,Arg,Tyr,Val, 0.708; Asn,Ala,Arg,Tyr,Ile,Trp, 0.708; Ser,Asn,Ala,Arg,Tyr,Val, 0.706; Ala,Cit,Tyr,Ile,Leu,Phe, 0.705; Arg,Tyr,Orn,Lys,Ile,Phe, 0.702; Ser,Ala,Arg,Pro,Tyr,Ile, 0.697; Ser,Ala,Arg,Tyr,Ile,Trp, 0.695; Gln,Ala,Arg,Tyr,Val,Trp, 0.695; Gln,Ala,Cit,Arg,Pro,Tyr, 0.687; Asn,Gly,Gln,Ala,Leu,Phe, 0.686; Gln,His,Ala,Cit,Val,Phe, 0.686; Ala,Cit,Val,Ile,Phe,Trp, 0.684; Ala,Cit,Val,Met,Ile,Phe, 0.684; Thr,Ala,Cit,Val,Leu,Phe, 0.683; Asn,Gly,Ala,Val,Lys,Phe, 0.683; Asn,Gly,His,Ala,Val,Phe, 0.678; His,Ala,Cit,Pro,Val,Phe, 0.678; Ala,Cit,Val,Lys,Ile,Phe, 0.678; Asn,Gly,Ala,Pro,Val,Phe, 0.676; Ala,Cit,Tyr,Met,Leu,Phe, 0.675; Asn,Gly,Ala,Pro,Leu,Phe, 0.67; Gly,Ala, Pro,Val,Met, Phe, 0.668; Ser,Ala,Cit,Tyr,Leu,Phe, 0.667; Ala,Cit,Tyr,Leu,Phe,Trp, 0.665; Gln,Ala,Cit,Tyr,Val,Phe, 0.663; Gly,Ala,Tyr,Val,Orn, Phe, 0.656

[306. Formulae with two explanatory variables obtained from amino acids and amino acid-related metabolites before the start of treatment with PFS as evaluation index]

Arg,Met, 0.784; Arg,NP, 0.779; Arg,KynA, 0.77; AnthA,NP, 0.77; hTrp,NP, 0.768; Arg,QA, 0.767; Arg,Kyn, 0.763; QA,NP, 0.76; Kyn,NP, 0.752; His,KynA, 0.74; Arg,AnthA, 0.737; Arg,hTrp, 0.737; Thr,NP, 0.737; Arg,hKyn, 0.737; Thr,Arg, 0.733; hKyn,NP, 0.732; Ser,Arg, 0.729; Arg,Trp, 0.727; Arg,Pro, 0.727; His,Arg, 0.727; Trp,NP, 0.727; Asn,Arg, 0.725; His,QA, 0.725; Gly,Arg, 0.725; His,Kyn, 0.724; Gln,Arg, 0.719; Ser,NP, 0.719; Met,NP, 0.719; KynA,NP, 0.717; Arg,Lys, 0.71; Ala,Arg, 0.71; Ala,Kyn, 0.71; His,AnthA, 0.71; Pro,NP, 0.706; Trp,KynA, 0.705; AnthA,KynA, 0.702; His,hKyn, 0.7; AnthA,QA, 0.698; hKyn,AnthA, 0.697; Gln,NP, 0.695; AnthA,Kyn, 0.694; Thr,KynA, 0.692; Asn,NP, 0.692; Ser,AnthA, 0.689; Pro,hKyn, 0.687; Ala,AnthA, 0.683; Ala,KynA, 0.679; Trp,Kyn, 0.678; Trp,hKyn, 0.678; Ala,NP, 0.678; Thr,hKyn, 0.676; Gly,NP, 0.675; Ala,hKyn, 0.675; Lys,hKyn, 0.67; hKyn,hTrp, 0.665; Lys,NP, 0.665; Thr,AnthA, 0.663; hTrp,QA, 0.663; Trp,AnthA, 0.663; hKyn,KynA, 0.663; Lys,AnthA, 0.662; Ala,QA, 0.662; Pro,Kyn, 0.66; Thr,QA, 0.657; Asn,Kyn, 0.656; Ser,hKyn, 0.654; Pro,AnthA, 0.652; hKyn,QA, 0.652; Met,hKyn, 0.652; Asn,hKyn, 0.651; Gln,QA, 0.649; Ser,Ala, 0.649; Asn,QA, 0.648; Lys,QA, 0.643; hKyn,Kyn, 0.641; Lys,KynA, 0.637; Ala,Met, 0.632; Asn,AnthA, 0.632; KynA,QA, 0.629; Gly,Trp, 0.627; Pro,QA, 0.627; Met,QA, 0.625; Gly,Ala, 0.624; Pro,KynA, 0.622; Gly,hKyn, 0.622; Gly,Lys, 0.621; Ala,hTrp, 0.621; Met,KynA, 0.619; Gln,Ala, 0.619; His,Ala, 0.617; Gly,QA, 0.617; Met,Kyn, 0.614; Thr,Ala, 0.613; Gly,AnthA, 0.613; Ala,Trp, 0.606; Asn,Ala, 0.603; Ala,Pro, 0.603; Lys,hTrp, 0.592; Ala,Lys, 0.592; Gln,hKyn, 0.587

[307. Formulae with three explanatory variables obtained from amino acids and amino acid-related metabolites before the start of treatment with PFS as evaluation index]

Arg,Met,NP, 0.825; Thr,Arg,QA, 0.797; Arg,QA,NP, 0.792; Arg,Trp,NP, 0.79; Arg,hTrp,NP, 0.789; Arg,Met,Kyn, 0.787; Arg,KynA,QA, 0.784; Arg,Kyn,NP, 0.784; Arg,Lys,NP, 0.783; Ser,Arg,NP, 0.783; Arg,hKyn,NP, 0.783; Gln,Arg,NP, 0.781; His,Arg,NP, 0.781; Asn,Arg,QA, 0.778; Arg,Pro,NP, 0.778; Ser,Arg,QA, 0.773; His,Arg,QA, 0.771; Arg,AnthA,Kyn, 0.771; Trp,AnthA,KynA, 0.771; Arg,hTrp,QA, 0.77; Gln,Arg,QA, 0.767; Arg,KynA,Kyn, 0.767; His,KynA,NP, 0.767; Arg,Lys,QA, 0.765; Arg,hTrp,Kyn, 0.763; Gly,Arg,QA, 0.763; Arg,hKyn,KynA, 0.763; Arg,hKyn,AnthA, 0.763; Ala,Arg,Kyn, 0.76; Ala,Arg,QA, 0.756; AnthA,KynA,NP, 0.756; Ala,Arg,NP, 0.749; Pro,hKyn,AnthA, 0.749; Arg,hKyn,hTrp, 0.746; Ala,KynA,NP, 0.746; Gly,Arg,hKyn, 0.744; Pro,hKyn,NP, 0.741; hKyn,AnthA,NP, 0.74; Thr,Arg,hKyn, 0.737; Ser,Arg,hKyn, 0.733; Arg,Trp,hKyn, 0.732; Ala,Kyn,NP, 0.73; Arg,Pro,hKyn, 0.729; His,hKyn,AnthA, 0.729; Arg,Lys,hKyn, 0.727; Ala,AnthA,QA, 0.724; Ala,AnthA,Kyn, 0.724; Trp,hKyn,AnthA, 0.724; Ala,Arg,hKyn, 0.722; Ala,hKyn,AnthA, 0.722; hKyn,AnthA,KynA, 0.719; Ala,KynA,Kyn, 0.719; Ala,hTrp,Kyn, 0.717; Lys,hKyn,AnthA, 0.716; Trp,hKyn,KynA, 0.713; Gly,Ala,Kyn, 0.71; Thr,hKyn,AnthA, 0.71; Ala,Kyn,QA, 0.708; Ala,hKyn,Kyn, 0.708; Ala,Trp,Kyn, 0.703; Ala,Lys,Kyn, 0.703; Ala,Met,Kyn, 0.703; Ser,Ala,Kyn, 0.702; Met,hKyn,AnthA, 0.698; Gly,Ala,NP, 0.697; Asn,hKyn,AnthA, 0.697; Ala,hKyn,KynA, 0.697; Ala,hTrp,NP, 0.695; Pro,Lys,hKyn, 0.689; Thr,Ala,KynA, 0.687; Gly,Lys,hKyn, 0.687; Ala,KynA,QA, 0.687; Asn,Ala,KynA, 0.686; Gly,Ala,KynA, 0.686; Lys,hKyn,KynA, 0.686; Lys,hKyn,NP, 0.686; Ala,hKyn,hTrp, 0.684; Ala,Lys,KynA, 0.683; Ala,hKyn,QA, 0.683; Gln,Ala,KynA, 0.683; His,Ala,KynA, 0.683; Ala,Met,KynA, 0.678; Ser,Ala,hKyn, 0.678; Lys,hKyn,hTrp, 0.678; Gly,Trp,hKyn, 0.676; Ala,Trp,hKyn, 0.676; Ala,Pro,hKyn, 0.676; Ala,Pro,KynA, 0.675; Ala,Met,hKyn, 0.675; Ser,Ala,KynA, 0.673; Gly,hKyn,AnthA, 0.673; Thr,Lys,hKyn, 0.671; Ala,Lys,hKyn, 0.671; Lys,hKyn,QA, 0.67; His,Ala,hKyn, 0.668; Gln,Ala,hKyn, 0.667; Gly,Ala,QA, 0.665; Gly,Ala,hKyn, 0.665; Gln,Lys,hKyn, 0.656; Gln,hKyn,AnthA, 0.646

[308. Formulae with four explanatory variables obtained from amino acids and amino acid-related metabolites before the start of treatment with PFS as evaluation index]

Ser,Arg,KynA,NP, 0.827; Arg,Pro,Met,NP, 0.825; Arg,Met,Trp,NP, 0.824; Arg,AnthA,KynA,NP, 0.822; Gly,Arg,Met,NP, 0.816; His,Arg,Met,NP, 0.814; Arg,Lys,KynA,NP, 0.813; Thr,Arg,KynA,NP, 0.81; Arg,AnthA,KynA,QA, 0.806; Arg,Met,AnthA,QA, 0.805; Arg,Met,Lys,NP, 0.803; Arg,hTrp,KynA,QA, 0.795; Arg,AnthA,KynA,Kyn, 0.79; Arg,hKyn,hTrp,NP, 0.781; Gly,Thr,Arg,QA, 0.779; Ala,AnthA,KynA,NP, 0.779; His,hKyn,AnthA,KynA, 0.775; Ala,Arg,AnthA,KynA, 0.775; Arg,Trp,hKyn,QA, 0.771; Arg,hKyn,hTrp,QA, 0.771; Ala,Arg,Kyn,NP, 0.771; Pro,hKyn,AnthA,KynA, 0.771; Thr,Arg,hKyn,AnthA, 0.768; Thr,Ala,Arg,Kyn, 0.767; Asn,Arg,hKyn,QA, 0.765; Arg,Pro,hKyn,NP, 0.765; Ser,Arg,hKyn,AnthA, 0.765; Ala,Arg,KynA,Kyn, 0.763; Ala,Arg,AnthA,NP, 0.763; Ala,Arg,Kyn,QA, 0.762; Ser,Ala,Arg,Kyn, 0.76; Gly,Ala,KynA,NP, 0.76; Arg,Lys,hKyn,NP, 0.759; Ala,hTrp,AnthA,KynA, 0.759; Ala,Arg,Lys,KynA, 0.757; Ala,Arg,Met,Lys, 0.756; Asn,Ala,Arg,KynA, 0.756; Ala,Arg,Lys,Kyn, 0.754; Ala,Arg,hKyn,AnthA, 0.754; Ser,Ala,AnthA,KynA, 0.754; Ala,Trp,AnthA,KynA, 0.754; Arg,Met,Lys,hKyn, 0.752; Ala,Arg,Met,hKyn, 0.752; Pro,hKyn,KynA,NP, 0.751; His,Ala,AnthA,KynA, 0.751; Gly,Ala,AnthA,KynA, 0.751; Ala,Arg,hKyn,KynA, 0.748; Ala,hTrp,KynA,NP, 0.748; Ala,AnthA,KynA,Kyn, 0.748; Ala,hKyn,AnthA,KynA, 0.748; Ala,Trp,KynA,NP, 0.743; Gln,Ala,KynA,NP, 0.741; Ser,Ala,KynA,NP, 0.741; His,Pro,hKyn,AnthA, 0.74; Ala,Met,AnthA,NP, 0.738; Pro,Trp,hKyn,AnthA, 0.738; Ala,Met,AnthA,KynA, 0.737; Thr,Ala,Arg,hKyn, 0.735; Arg,Lys,hKyn,hTrp, 0.733; Ala,Trp,AnthA,Kyn, 0.732; Gly,Ala,KynA,Kyn, 0.732; Thr,Ala,AnthA,Kyn, 0.73; Ala,hKyn,AnthA,QA, 0.73; Ala,hKyn,hTrp,AnthA, 0.73; Trp,hKyn,hTrp,AnthA, 0.729; Thr,Ala,hKyn,AnthA, 0.725; Gln,Ala,AnthA,Kyn, 0.724; Gly,Ala,AnthA,Kyn, 0.724; Trp,hKyn,AnthA,Kyn, 0.724; Lys,hKyn,AnthA,NP, 0.724; Gln,Ala,Arg,hKyn, 0.722; Ala,Lys,KynA,Kyn, 0.722; Ala,Lys,hKyn,AnthA, 0.722; Ala,hTrp,Kyn,QA, 0.721; His,Ala,AnthA,Kyn, 0.721; Ala,Trp,KynA,Kyn, 0.719; Ala,KynA,Kyn,QA, 0.719; His,Lys,hKyn,AnthA, 0.717; Lys,hKyn,AnthA,QA, 0.717; Gly,Ala,hKyn,AnthA, 0.717; Ala,Lys,hTrp,Kyn, 0.716; Asn,Ala,KynA,Kyn, 0.716; Thr,Lys,hKyn,AnthA, 0.716; Ala,Lys,hKyn,Kyn, 0.714; Ala,Met,KynA,Kyn, 0.714; Asn,Gly,Ala,Kyn, 0.708; Gly,Ala,KynA,QA, 0.708; Ala,hKyn,KynA,QA, 0.708; Gly,Ala,Met,Kyn, 0.702; Asn,Ala,hKyn,KynA, 0.702; Ala,Pro,hKyn,KynA, 0.697; Thr,Ala,hKyn,KynA, 0.697; Thr,Ala,KynA,QA, 0.694; Gly,Ala,hTrp,KynA, 0.694; Ala,Met,hKyn,KynA, 0.694; Thr,Ala,Met,hKyn, 0.687; Gln,Ala,hTrp,KynA, 0.687; Ala,Trp,KynA,QA, 0.687; Ala,Met,KynA,QA, 0.687; Gly,Ala,Lys,hKyn, 0.675

[309. Formulae with five explanatory variables obtained from amino acids and amino acid-related metabolites before the start of treatment with PFS as evaluation index]

Arg,Pro,hKyn,AnthA,KynA, 0.808; Arg,hKyn,AnthA,KynA,NP, 0.805; Asn,Ala,Arg,Met,NP, 0.805; Ala,Arg,Met,Trp,NP, 0.803; Arg,Pro,Met,hKyn,NP, 0.802; Ala,Arg,Met,AnthA,Kyn, 0.797; Ala,Arg,Met,Trp,Kyn, 0.794; Thr,Ala,Arg,KynA,NP, 0.794; Thr,Ala,Arg,AnthA,KynA, 0.792; His,Ala,Arg,Met,Kyn, 0.789; Gln,Ala,Arg,KynA,NP, 0.789; Gly,Ala,AnthA,KynA,NP, 0.781; Gly,Ala,Arg,Met,Kyn, 0.781; Ala,Arg,Met,Trp,KynA, 0.781; Ala,Arg,AnthA,Kyn,NP, 0.781; Ala,Arg,Met,hKyn,NP, 0.779; Gly,Arg,hKyn,AnthA,NP, 0.779; Arg,hKyn,AnthA,Kyn,QA, 0.779; Thr,Ala,Arg,KynA,QA, 0.778; Ala,Arg,Met,Lys,KynA, 0.778; Asn,Arg,hKyn,AnthA,QA, 0.778; Ala,Arg,Met,hTrp,KynA, 0.776; Asn,Ala,Arg,AnthA,KynA, 0.775; Arg,Met,Lys,hKyn,AnthA, 0.775; Asn,Trp,hKyn,AnthA,KynA, 0.775; Ala,Arg,Pro,KynA,NP, 0.775; Ala,Arg,Trp,Kyn,NP, 0.775; Asn,Ala,Arg,KynA,Kyn, 0.773; Ala,Lys,hTrp,AnthA,KynA, 0.768; Arg,Lys,hKyn,AnthA,Kyn, 0.768; His,hKyn,AnthA,KynA,NP, 0.768; Ala,hKyn,AnthA,KynA,NP, 0.767; Ala,Arg,hKyn,AnthA,KynA, 0.767; Ala,hTrp,AnthA,KynA,QA, 0.763; Ala,Arg,hTrp,KynA,Kyn, 0.763; Ala,hKyn,hTrp,AnthA,KynA, 0.762; Ser,Trp,hKyn,AnthA,KynA, 0.762; Ala,Arg,Met,Trp,hKyn, 0.762; Ala,Met,AnthA,KynA,NP, 0.76; Thr,Ala,hTrp,AnthA,KynA, 0.759; Ala,Arg,hKyn,KynA,QA, 0.759; Ala,Arg,Met,hKyn,hTrp, 0.759; Ala,Arg,hKyn,hTrp,AnthA, 0.757; Ala,hTrp,AnthA,KynA,Kyn, 0.756; Asn,Ala,Arg,hKyn,KynA, 0.756; Gln,Ala,KynA,Kyn,NP, 0.756; Arg,Pro,Lys,hKyn,AnthA, 0.756; Thr,Ala,Arg,hKyn,NP, 0.754; Ala,Trp,AnthA,KynA,QA, 0.752; Ala,hKyn,AnthA,KynA,QA, 0.751; Ser,Gln,Ala,AnthA,KynA, 0.751; Gln,Thr,Ala,KynA,NP, 0.751; Gly,Arg,Lys,hKyn,AnthA, 0.749; Ala,hKyn,AnthA,KynA,Kyn, 0.748; Gly,Ala,AnthA,KynA,Kyn, 0.748; Ser,Pro,Lys,hKyn,AnthA, 0.748; Gly,Pro,hKyn,AnthA,NP, 0.748; Gly,Ala,Arg,Kyn,QA, 0.748; Ala,Pro,AnthA,KynA,Kyn, 0.744; His,Ala,Met,KynA,Kyn, 0.743; Gly,Ala,hTrp,Kyn,NP, 0.743; Gly,Ala,Arg,hKyn,QA, 0.743; Asn,Arg,Met,Lys,hKyn, 0.743; His,Ala,hKyn,AnthA,NP, 0.741; Ala,hKyn,hTrp,AnthA,QA, 0.738; Ala,Met,hTrp,KynA,NP, 0.735; Ser,Ala,AnthA,KynA,Kyn, 0.733; Gly,Ala,hTrp,KynA,Kyn, 0.733; Ala,Met,hKyn,KynA,NP, 0.733; Gln,Ala,hTrp,AnthA,Kyn, 0.733; His,Ala,hTrp,AnthA,Kyn, 0.733; Ser,Ala,Lys,hKyn,AnthA, 0.733; Thr,Ala,hTrp,KynA,Kyn, 0.729; Ser,Gln,Ala,hKyn,AnthA, 0.729; Ala,Met,hKyn,hTrp,AnthA, 0.727; Ala,Lys,Trp,hKyn,AnthA, 0.727; Gly,Ala,Pro,KynA,Kyn, 0.725; Gly,Ala,hKyn,AnthA,QA, 0.725; Gln,Ala,hKyn,AnthA,QA, 0.725; Thr,Ala,Lys,hKyn,AnthA, 0.724; His,Thr,Ala,KynA,Kyn, 0.722; Asn,Thr,Ala,KynA,Kyn, 0.721; Thr,Ala,Pro,KynA,Kyn, 0.721; Gln,Ala,Lys,hKyn,AnthA, 0.721; Gln,Ala,Pro,KynA,Kyn, 0.719; Gln,Ala,Met,KynA,NP, 0.719; Ser,Gly,Ala,hKyn,AnthA, 0.716; Ser,Ala,hTrp,KynA,Kyn, 0.713; Ser,Thr,Ala,hKyn,KynA, 0.71; Ala,Met,Lys,hKyn,AnthA, 0.706; Ala,Pro,hKyn,KynA,QA, 0.705; His,Ala,Met,hKyn,KynA, 0.703; Thr,Ala,hKyn,hTrp,KynA, 0.702; Asn,His,Ala,hKyn,KynA, 0.702; His,Ala,Trp,hKyn,KynA, 0.702; Asn,Ala,hTrp,KynA,QA, 0.702; Ser,Ala,hKyn,KynA,QA, 0.7; Ala,Met,hTrp,KynA,QA, 0.7; Ser,Ala,Trp,hKyn,KynA, 0.698; Gly,Ala,Met,hKyn,KynA, 0.697

[310. Formulae with six explanatory variables obtained from amino acids and amino acid-related metabolites before the start of treatment with PFS as evaluation index]

Ala,Arg,Met,KynA,QA,NP, 0.814; Asn,Ala,Arg,Lys,KynA,NP, 0.808; Ala,Arg,Pro,Met,Lys,NP, 0.802; Ala,Arg,Met,Lys,KynA,QA, 0.798; Ser,His,Ala,Arg,KynA,NP, 0.798; Gly,Ala,hTrp,AnthA,KynA,NP, 0.797; Ala,Arg,Met,hTrp,Kyn,QA, 0.795; Ala,Arg,Met,Lys,Trp,NP, 0.795; His,Ala,Arg,KynA,Kyn,NP, 0.794; Ala,Arg,Met,Trp,AnthA,QA, 0.794; Thr,Ala,Met,AnthA,KynA,NP, 0.792; Ser,Gly,Ala,Arg,Met,Kyn, 0.784; His,Ala,Arg,Met,hKyn,Kyn, 0.784; Ser,Asn,Ala,Arg,AnthA,Kyn, 0.784; Ala,Arg,Met,hKyn,KynA,QA, 0.783; Pro,Lys,hKyn,AnthA,KynA,Kyn, 0.781; Pro,hKyn,AnthA,KynA,Kyn,NP, 0.781; Arg,Trp,hKyn,hTrp,AnthA,KynA, 0.781; Asn,Gly,Ala,Arg,AnthA,Kyn, 0.781; Asn,Gly,Ala,AnthA,KynA,NP, 0.779; Ser,Arg,Trp,hKyn,AnthA,KynA, 0.779; Asn,Ala,Arg,hTrp,AnthA,KynA, 0.778; His,Ala,Arg,hKyn,AnthA,Kyn, 0.778; Asn,Arg,Trp,hKyn,AnthA,KynA, 0.778; Gly,Ala,Arg,Met,hTrp,KynA, 0.778; Ala,Lys,hKyn,AnthA,KynA,NP, 0.776; Gln,Ala,Lys,AnthA,KynA,NP, 0.776; Ala,Pro,hTrp,AnthA,KynA,QA, 0.776; Asn,Ala,Trp,AnthA,KynA,Kyn, 0.775; Asn,Ala,Trp,hKyn,AnthA,KynA, 0.775; Ala,Arg,Pro,hTrp,AnthA,KynA, 0.775; Ser,Asn,Ala,Arg,AnthA,KynA, 0.775; Thr,Arg,Lys,hKyn,AnthA,KynA, 0.775; Thr,Ala,Arg,Pro,Met,KynA, 0.775; Ala,Arg,Met,Lys,hKyn,NP, 0.771; His,Ala,Pro,AnthA,KynA,QA, 0.771; His,Ala,Arg,Lys,KynA,Kyn, 0.771; Pro,hTrp,AnthA,KynA,QA,NP, 0.771; Gln,Pro,hKyn,AnthA,KynA,QA, 0.77; Asn,Gly,Ala,Trp,AnthA,KynA, 0.765; Asn,Ala,Arg,hKyn,hTrp,AnthA, 0.765; Ala,Arg,Lys,hKyn,AnthA,Kyn, 0.765; Gln,Ala,Arg,hKyn,KynA,Kyn, 0.763; Ser,Asn,Ala,AnthA,KynA,QA, 0.763; Asn,Gly,Ala,hTrp,AnthA,KynA, 0.763; Gly,Arg,Pro,Met,Lys,hKyn, 0.763; Gln,Ala,hTrp,KynA,Kyn,NP, 0.762; Gly,Ala,Lys,AnthA,KynA,QA, 0.762; Gly,Gln,Trp,hKyn,AnthA,KynA, 0.762; Gly,Ala,Arg,hKyn,AnthA,NP, 0.762; Ala,Lys,hKyn,hTrp,AnthA,KynA, 0.76; Ala,Arg,Trp,hKyn,AnthA,NP, 0.76; Ala,hTrp,AnthA,KynA,Kyn,QA, 0.759; Asn,Ala,hKyn,hTrp,AnthA,KynA, 0.759; Pro,Met,Lys,hKyn,hTrp,AnthA, 0.759; Asn,Gly,Thr,Ala,KynA,NP, 0.759; Thr,Ala,Arg,Pro,hKyn,AnthA, 0.759; Ser,Thr,Trp,hKyn,AnthA,KynA, 0.757; Gly,Ala,hTrp,AnthA,KynA,Kyn, 0.756; Asn,Ala,hTrp,AnthA,KynA,Kyn, 0.754; Gly,Thr,Ala,Arg,hKyn,AnthA, 0.754; Ala,Met,Lys,AnthA,KynA,QA, 0.752; Ser,Ala,hKyn,hTrp,AnthA,KynA, 0.749; Asn,Ala,AnthA,KynA,Kyn,QA, 0.749; Gly,Ala,Trp,hKyn,AnthA,KynA, 0.749; Thr,Ala,hTrp,AnthA,KynA,Kyn, 0.748; Thr,Ala,hKyn,AnthA,KynA,Kyn, 0.748; Ala,Pro,AnthA,KynA,Kyn,QA, 0.748; Ala,Pro,Lys,AnthA,KynA,Kyn, 0.746; Gly,Thr,Ala,AnthA,KynA,Kyn, 0.746; Ser,Gln,Ala,hTrp,KynA,NP, 0.746; Asn,Gln,Ala,hTrp,KynA,NP, 0.746; Ala,Trp,hKyn,KynA,Kyn,NP, 0.744; Ser,Gly,Ala,hTrp,KynA,NP, 0.743; Asn,Gln,Ala,KynA,Kyn,NP, 0.743; Ser,Ala,hTrp,AnthA,KynA,Kyn, 0.741; Asn,Ala,hKyn,AnthA,KynA,Kyn, 0.741; Gly,Gln,Ala,hKyn,AnthA,KynA, 0.741; His,Ala,Met,hTrp,KynA,Kyn, 0.741; Gln,His,Ala,Met,KynA,Kyn, 0.741; Gln,His,Ala,AnthA,KynA,Kyn, 0.74; His,Ala,hKyn,hTrp,KynA,Kyn, 0.74; Gln,Ala,Pro,hTrp,KynA,Kyn, 0.74; Ala,Met,hTrp,AnthA,KynA,Kyn, 0.738; Ser,Thr,Ala,AnthA,KynA,Kyn, 0.737; Gly,Ala,hKyn,hTrp,AnthA,Kyn, 0.737; Gly,Ala,Met,KynA,QA,NP, 0.737; Ser,Gly,Ala,Trp,KynA,NP, 0.735; Gly,His,Ala,hTrp,KynA,Kyn, 0.733; Asn,Ala,Lys,hTrp,KynA,Kyn, 0.729; Asn,Thr,Ala,hKyn,KynA,Kyn, 0.727; Thr,Ala,Met,hKyn,AnthA,Kyn, 0.727; Asn,Ala,Trp,hTrp,KynA,Kyn, 0.725; Asn,Gln,Ala,hTrp,KynA,Kyn, 0.724; Ala,Met,hKyn,hTrp,KynA,Kyn, 0.724; Asn,Thr,Ala,KynA,Kyn,QA, 0.724; Gly,Thr,Ala,Met,KynA,Kyn, 0.719; Asn,Gly,Ala,hKyn,AnthA,QA, 0.716; His,Ala,Met,hKyn,hTrp,KynA, 0.711; Gly,Gln,Ala,hKyn,hTrp,KynA, 0.705

[311. Formulae with two explanatory variables obtained from amino acids before the start of treatment with the response rate as evaluation index]

Arg,Met, 0.691; Ala,Phe, 0.686; Gly,Arg, 0.681; Ala,Orn, 0.676; Ser,Arg, 0.672; Thr,Arg, 0.671; Gln,Arg, 0.666; Ala,Arg, 0.666; Ala,Met, 0.664; Asn,Ala, 0.66; Arg,Orn, 0.652; Ala,Lys, 0.652; Arg,Ile, 0.65; His,Arg, 0.65; Ala,Cit, 0.65; Gly,Orn, 0.649; Ala,Ile, 0.647; His,Ala, 0.647; Arg,Val, 0.645; Arg,Pro, 0.642; Arg,Tyr, 0.642; Ala,Val, 0.642; Thr,Ala, 0.642; Ala,Pro, 0.64; Arg,Phe, 0.64; Arg,Lys, 0.639; Tyr,Orn, 0.639; Ala,Leu, 0.639; Ala,Tyr, 0.639; Gln,Ala, 0.637; Met,Orn, 0.635; Pro,Orn, 0.633; Cit,Arg, 0.633; Ala,Trp, 0.633; Ser,Ala, 0.633; Ser,Orn, 0.632; Cit,Orn, 0.63; Gln,Orn, 0.628; Asn,Orn, 0.627; Orn,Phe, 0.627; Lys,Phe, 0.625; Orn,Lys, 0.623; Gly,Ala, 0.623; Val,Phe, 0.62; Pro,Phe, 0.62; Orn,Trp, 0.618; Val,Orn, 0.615; Met,Trp, 0.615; Phe,Trp, 0.615; Orn,Leu, 0.611; His,Orn, 0.611; Thr,Orn, 0.611; Cit,Phe, 0.61; Orn,Ile, 0.606; Thr,Phe, 0.606; Cit,Pro, 0.606; Gly,Cit, 0.605; Tyr,Trp, 0.6; Tyr,Phe, 0.596; Thr,Lys, 0.595; Asn,Cit, 0.591; Gly,Trp, 0.588; Cit,Tyr, 0.586; Thr,Cit, 0.586; Cit,Trp, 0.586; Gly,Lys, 0.584; Met,Lys, 0.584; Pro,Trp, 0.584; Ser,Lys, 0.581; Ile,Trp, 0.581; Thr,Pro, 0.579; Leu,Phe, 0.579; Asn,Lys, 0.574; Val,Lys, 0.574; Val,Trp, 0.574; Gln,Trp, 0.573; Cit,Ile, 0.571; Gln,Lys, 0.571; His,Trp, 0.571; Lys,Trp, 0.571; Leu,Trp, 0.571; Tyr,Lys, 0.569; His,Lys, 0.569; Lys,Leu, 0.569; Lys,Ile, 0.569; Thr,Trp, 0.566; Gly,Thr, 0.564

[312. Formulae with three explanatory variables obtained from amino acids before the start of treatment with the response rate as evaluation index]

Ala,Orn,Phe, 0.743; Ala,Arg,Met, 0.738; Ser,Arg,Met, 0.715; Gly,Arg,Pro, 0.709; Gly,Arg,Leu, 0.708; Gly,Arg,Ile, 0.708; Asn,Ala,Cit, 0.708; Arg,Val,Met, 0.708; Ala,Cit,Met, 0.708; Ala,Arg,Phe, 0.704; Arg,Pro,Met, 0.703; Asn,Ala,Orn, 0.701; Gly,Thr,Arg, 0.699; Ala,Leu,Phe, 0.696; Pro,Orn,Phe, 0.694; Arg,Met,Orn, 0.693; Ala,Met,Phe, 0.693; Ala,Val,Orn, 0.693; Arg,Tyr,Met, 0.691; Asn,Ala,Arg, 0.689; Gly,His,Arg, 0.688; Gln,Arg,Phe, 0.688; Ser,Ala,Phe, 0.688; Thr,Arg,Trp, 0.686; Gly,Arg,Orn, 0.686; Ala,Lys,Phe, 0.686; Ala,Orn,Ile, 0.686; Thr,Ala,Orn, 0.686; Gly,Gln,Arg, 0.684; Ala,Val,Phe, 0.684; His,Ala,Arg, 0.682; Ala,Phe,Trp, 0.682; Gln,Arg,Pro, 0.681; Ser,Arg,Ile, 0.681; Ala,Arg,Val, 0.681; Thr,Ala,Phe, 0.681; Ala,Tyr,Phe, 0.679; Ser,Ala,Arg, 0.679; Ser,Arg,Pro, 0.677; Ala,Cit,Orn, 0.677; Ala,Ile,Phe, 0.677; Ala,Cit,Lys, 0.674; Ala,Pro,Orn, 0.674; Ala,Pro,Phe, 0.674; Gln,Ala,Phe, 0.674; Ser,Asn,Arg, 0.672; His,Ala,Orn, 0.672; Asn,Ala,Met, 0.671; Ala,Arg,Leu, 0.669; Ser,Cit,Arg, 0.667; Ala,Cit,Arg, 0.667; Val,Orn,Phe, 0.666; Gly,Ala,Arg, 0.666; Ala,Arg,Pro, 0.664; Gln,Ala,Cit, 0.664; Gly,Phe,Trp, 0.662; Ala,Arg,Trp, 0.662; Ala,Tyr,Met, 0.662; Gln,Ala,Met, 0.662; Asn,Gln,Arg, 0.66; Ala,Cit,Ile, 0.66; Ala,Tyr,Trp, 0.659; His,Ala,Cit, 0.659; Asn,Arg,Phe, 0.657; Ser,Ala,Met, 0.657; Arg,Val,Phe, 0.657; Ser,Asn,Ala, 0.655; Tyr,Orn,Phe, 0.654; Asn,Arg,Trp, 0.654; His,Thr,Ala, 0.654; Ala,Cit,Val, 0.654; Asn,Ala,Leu, 0.654; Ala,Cit,Leu, 0.652; Asn,Ala,Trp, 0.65; Pro,Val,Phe, 0.649; Arg,Pro,Tyr, 0.649; Arg,Lys,Phe, 0.649; Arg,Ile,Phe, 0.647; Asn,Gln,Ala, 0.647; Ala,Leu,Trp, 0.642; Ala,Lys,Trp, 0.642; His,Ala,Leu, 0.64; His,Ala,Tyr, 0.64; Gln,Ala,Ile, 0.639; Ser,Ala,Val, 0.639; Cit,Arg,Phe, 0.639; Ser,Ala,Lys, 0.637; Gly,Ala,Leu, 0.633; His,Phe,Trp, 0.632; Val,Phe,Trp, 0.632; Gln,Ala,Lys, 0.632; Ser,His,Ala, 0.63; Ser,Ala,Trp, 0.627; Gln,Ala,Pro, 0.627; Asn,Gly,Ala, 0.627; Gly,Ala,Val, 0.625; His,Ala,Trp, 0.623; Ala,Tyr,Leu, 0.623

[313. Formulae with four explanatory variables obtained from amino acids before the start of treatment with the response rate as evaluation index]

Ser,Ala,Orn,Phe, 0.794; Gln,Ala,Orn,Phe, 0.789; Ala,Arg,Met,Leu, 0.765; Gly,Arg,Pro,Met, 0.76; Thr,Ala,Orn,Phe, 0.758; Ala,Orn,Lys,Phe, 0.755; Ala,Pro,Orn,Phe, 0.753; His,Ala,Arg,Met, 0.748; Ala,Arg,Pro,Met, 0.748; Ala,Orn,Ile,Phe, 0.747; Ala,Arg,Met,Ile, 0.745; Ala,Arg,Orn,Phe, 0.743; Ala,Ile,Leu,Phe, 0.738; Thr,Ala,Arg,Met, 0.736; Ala,Arg,Met,Phe, 0.735; Gly,Ala,Arg,Phe, 0.735; Ala,Met,Orn,Leu, 0.733; Ala,Pro,Met,Orn, 0.733; Asn,Ala,Arg,Met, 0.731; Thr,Ala,Met,Orn, 0.73; Ala,Met,Orn,Trp, 0.728; Arg,Met,Leu,Phe, 0.726; Asn,Ala,Arg,Phe, 0.726; Arg,Pro,Met,Leu, 0.726; Ala,Arg,Tyr,Orn, 0.723; Asn,Ala,Cit,Phe, 0.723; Pro,Val,Orn,Phe, 0.718; Thr,Ala,Cit,Met, 0.718; His,Arg,Met,Trp, 0.718; Ser,Arg,Met,Phe, 0.716; Arg,Pro,Val,Met, 0.716; Gln,Ala,Arg,Tyr, 0.715; Gln,Arg,Met,Trp, 0.715; His,Ala,Met,Orn, 0.713; Ala,Tyr,Orn,Ile, 0.711; Arg,Met,Orn,Trp, 0.711; Ala,Cit,Val,Met, 0.711; Asn,Gly,Ala,Arg, 0.711; Asn,Gly,Ala,Orn, 0.711; Ala,Cit,Met,Ile, 0.711; Gly,Arg,Orn,Phe, 0.711; Thr,Arg,Met,Trp, 0.709; Ala,Cit,Met,Leu, 0.709; Ala,Arg,Leu,Phe, 0.708; Gly,Ala,Arg,Lys, 0.708; Ser,Ala,Met,Orn, 0.708; Ala,Arg,Phe,Trp, 0.704; Cit,Arg,Pro,Met, 0.704; Asn,Ala,Val,Phe, 0.704; Ala,Cit,Tyr,Met, 0.703; Ala,Cit,Pro,Met, 0.703; Gln,Ala,Orn,Ile, 0.703; Ala,Val,Lys,Phe, 0.701; Arg,Pro,Tyr,Met, 0.701; Asn,Ala,Orn,Trp, 0.701; His,Ala,Tyr,Orn, 0.701; Gly,Ala,Cit,Met, 0.701; Gln,Ala,Orn,Lys, 0.701; Thr,Arg,Val,Phe, 0.699; Ser,Cit,Arg,Phe, 0.699; Ala,Val,Met,Phe, 0.699; Ala,Tyr,Orn,Lys, 0.699; Asn,Ala,Cit,Orn, 0.699; Asn,Ala,Orn,Leu, 0.699; Gln,Arg,Pro,Met, 0.699; Gln,His,Ala,Orn, 0.699; Asn,Ala,Orn,Ile, 0.698; Gly,Cit,Arg,Phe, 0.698; Gln,Ala,Pro,Orn, 0.696; Ser,Ala,Cit,Phe, 0.696; Ser,His,Ala,Orn, 0.694; Ala,Leu,Phe,Trp, 0.693; Gly,Thr,Arg,Phe, 0.693; Gly,Arg,Ile,Phe, 0.693; Gly,Arg,Leu,Phe, 0.693; Ser,Gln,Arg,Phe, 0.691; Gly,Ala,Leu,Phe, 0.691; Ala,Cit,Lys,Phe, 0.689; Ala,Cit,Tyr,Phe, 0.688; Gln,Arg,Orn,Phe, 0.688; Ser,Ala,Arg,Orn, 0.688; Ser,Ala,Val,Orn, 0.686; Gly,Ala,Tyr,Orn, 0.686; His,Ala,Val,Orn, 0.682; Gly,Thr,Ala,Orn, 0.681; Ala,Cit,Ile,Phe, 0.679; Gly,Ala,Orn,Leu, 0.679; Ser,Ala,Orn,Ile, 0.677; Ser,Ala,Phe,Trp, 0.677; Ala,Met,Leu,Trp, 0.676; Ser,Arg,Pro,Phe, 0.676; Ser,Ala,Tyr,Orn, 0.672; Gln,Ala,Leu,Phe, 0.671; Ala,Met,Lys,Trp, 0.669; Ser,Ala,Orn,Trp, 0.669; Gln,Ala,Met,Trp, 0.667; His,Ala,Met,Trp, 0.667; Arg,Orn,Phe,Trp, 0.662; Ser,Ala,Pro,Orn, 0.657; Thr,Ala,Met,Trp, 0.655

[314. Formulae with five explanatory variables obtained from amino acids before the start of treatment with the response rate as evaluation index]

Ala,Met,Orn,Leu,Phe, 0.813; Gly,Ala,Orn,Phe,Trp, 0.802; Gly,Thr,Ala,Orn,Phe, 0.802; Gly,Ala,Orn,Ile,Phe, 0.799; Gly,His,Ala,Orn, Phe, 0.799; Gly,Ala,Cit,Orn, Phe, 0.797; Gly,Ala,Orn,Leu, Phe, 0.797; Gln,Ala, Pro,Orn, Phe, 0.796; Gly,Gln,Ala,Orn, Phe, 0.796; Ser,Ala,Orn,Lys, Phe, 0.794; Gly,Ala,Orn,Lys, Phe, 0.794; Gly,Ala,Arg,Orn, Phe, 0.791; Asn,Ala,Orn,Phe,Trp, 0.784; Ala,Orn,Ile,Leu,Phe, 0.784; His,Ala,Pro,Orn,Phe, 0.78; Gln,Ala,Arg,Orn,Phe, 0.78; His,Ala,Cit,Orn, Phe, 0.779; Ser,Ala,Arg,Orn, Phe, 0.779; His,Ala,Orn,Lys,Phe, 0.774; Asn,Ala,Arg,Met,Leu, 0.77; Asn,Ala,Orn,Lys,Phe, 0.77; His,Ala,Arg,Met,Leu, 0.767; Ala,Cit,Orn,Leu,Phe, 0.764; Ala,Arg,Orn,Leu,Phe, 0.758; Gly,Cit,Arg,Pro,Met, 0.753; Thr,Ala,Arg,Met,Orn, 0.752; Asn,Ala,Cit,Leu,Phe, 0.75; Ala,Pro,Orn,Ile,Phe, 0.747; Ala,Arg,Met,Ile,Phe, 0.747; Gly,His,Ala,Arg,Phe, 0.745; Thr,Ala,Met,Orn,Leu, 0.743; Ala,Arg,Val,Met,Phe, 0.743; Gly,Ala,Arg,Ile,Phe, 0.742; Ala,Met,Ile,Leu,Phe, 0.74; Asn,Ala,Arg,Met,Lys, 0.738; Ala,Cit,Val,Met,Phe, 0.738; Asn,Ala,Arg,Met,Trp, 0.738; Gln,Ala,Arg,Met,Trp, 0.738; Ser,Asn,Ala,Arg,Met, 0.738; Gln,Ala,Ile,Leu,Phe, 0.736; Ala,Met,Orn,Lys,Leu, 0.736; Ala,Pro,Met,Leu,Phe, 0.735; His,Thr,Ala,Met,Orn, 0.733; Gln,Ala,Arg,Met,Phe, 0.733; Ser, Pro, Orn, Lys, Phe, 0.73; Asn,Ala,Met, Orn, Leu, 0.73; Gln,Arg,Met,Leu,Phe, 0.726; Asn,Ala,Arg,Tyr,Phe, 0.726; Asn,Arg,Pro,Met,Leu, 0.726; Gly,Ala,Cit,Met,Phe, 0.726; Gly,Arg,Tyr,Met,Trp, 0.725; Ser,Arg,Pro,Val,Met, 0.723; Gly,Gln,Arg,Met,Phe, 0.72; Ser,Arg,Orn,Lys,Phe, 0.72; Ala,Cit,Lys,Leu,Phe, 0.718; Asn,Ala,Cit,Met,Phe, 0.718; Ala,Cit,Met,Orn,Lys, 0.718; Asn,Gly,Ala,Phe,Trp, 0.718; Ser,Gln,Arg,Orn,Phe, 0.716; Asn,Ala,Met,Orn,Lys, 0.716; Thr,Ala,Arg,Pro,Phe, 0.715; His,Arg,Pro,Val,Met, 0.715; Ala,Cit,Val,Met,Orn, 0.715; Thr,Ala,Cit,Arg,Phe, 0.713; Asn,Gly,Ala,Orn,Ile, 0.713; Gly,Cit,Arg,Pro,Phe, 0.713; Asn,Gln,Ala,Orn,Trp, 0.713; Ser,His,Ala,Arg,Phe, 0.713; Arg,Pro,Tyr,Val,Met, 0.711; Arg,Pro,Tyr,Met,Phe, 0.711; Ser,His,Ala,Arg,Tyr, 0.711; Gly,Gln,Ala,Met,Orn, 0.711; Gly,Thr,Arg,Orn,Phe, 0.709; Gly,Ala,Cit,Met,Leu, 0.709; Asn,Ala,Tyr,Orn,Lys, 0.708; Gly,Gln,Ala,Tyr,Orn, 0.708; Gly,Ala,Cit,Met,Orn, 0.708; Ser,Thr,Arg,Phe,Trp, 0.706; Asn,Ala,Arg,Orn,Lys, 0.706; Ser,Asn,Ala,Leu,Phe, 0.706; Asn,Ala,Tyr, Phe,Trp, 0.706; Asn, Pro,Orn, Phe,Trp, 0.704; Ala,Cit,Tyr,Leu,Phe, 0.704; His,Ala,Val,Met,Phe, 0.704; Ser,Ala,Met,Orn,Lys, 0.704; Ser,Asn,Arg,Phe,Trp, 0.703; Ser,Thr,Arg,Val,Phe, 0.703; Gly,Ala,Leu,Phe,Trp, 0.703; Ala,Cit,Met,Phe,Trp, 0.703; Arg,Val,Met,Phe,Trp, 0.701; Ser,Asn,Thr,Ala,Orn, 0.701; Gly,Ala,Val,Met,Orn, 0.701; Ser,Asn,Ala,Orn,Lys, 0.699; Arg,Pro,Val,Met,Orn, 0.699; Asn,Ala,Tyr,Orn,Leu, 0.699; Gly,Ala,Lys,Phe,Trp, 0.699; Ser,Cit,Arg,Leu,Phe, 0.698; Ser,Arg,Tyr,Val,Phe, 0.698; Gly,Arg,Leu,Phe,Trp, 0.696; Ser,His,Ala,Tyr,Orn, 0.689

[315. Formulae with six explanatory variables obtained from amino acids before the start of treatment with the response rate as evaluation index]

Ala,Val,Met,Orn,Leu,Phe, 0.834; Gly,Ala,Met,Orn,Leu,Phe, 0.818; Ala,Arg,Met,Orn,Leu,Phe, 0.816; Ser,Ala,Met, Orn, Leu, Phe, 0.816; Ala, Pro,Met, Orn, Leu, Phe, 0.816; Ala,Met,Orn,Leu,Phe,Trp, 0.816; Ala,Met,Orn,Lys,Leu,Phe, 0.814; Thr,Ala,Met,Orn,Leu,Phe, 0.813; Ser,Gln,Ala,Tyr,Orn,Phe, 0.807; Gly,Ala,Val, Orn, Leu, Phe, 0.806; Ala,Met, Orn, Ile, Leu, Phe, 0.804; Gly,Gln,Ala,Pro,Orn,Phe, 0.804; Gly,His,Ala,Orn,Phe,Trp, 0.801; Gly,His,Ala,Tyr,Orn,Phe, 0.801; Ser,Gly,Ala,Met,Orn,Phe, 0.801; Gly,Gln,Ala,Orn,Lys,Phe, 0.801; Gly,Ala,Val,Met,Orn,Phe, 0.801; Ser,Ala,Cit,Val,Orn,Phe, 0.801; Ser,Ala,Cit,Orn,Ile,Phe, 0.801; Gly,Ala,Val,Orn,Phe,Trp, 0.799; Gly,His,Ala,Met,Orn,Phe, 0.797; Thr,Ala,Val,Orn,Leu,Phe, 0.797; Gln,Ala,Val,Orn,Leu,Phe, 0.797; Gly,His,Ala,Val,Orn,Phe, 0.796; Gln,Ala,Val,Met,Orn,Phe, 0.796; His,Ala,Pro,Met,Orn,Phe, 0.796; Ser,Ala,Arg,Met,Orn,Phe, 0.794; Ala,Arg,Val,Met,Leu,Phe, 0.791; Ala,Cit,Val,Met,Orn,Leu, 0.791; Ser,Thr,Ala,Orn,Phe,Trp, 0.791; His,Ala, Pro,Val,Orn, Phe, 0.789; Asn,Ala,Arg, Pro,Orn, Phe, 0.789; Gln,Ala,Arg,Met,Leu,Phe, 0.787; His,Ala,Val,Orn,Leu,Phe, 0.785; Ala,Arg,Orn,Ile,Leu,Phe, 0.785; His,Ala,Val,Orn,Ile,Phe, 0.784; Asn,Ala,Tyr,Val,Orn,Phe, 0.782; Asn,Ala,Orn,Lys,Phe,Trp, 0.782; Ala,Tyr,Val,Orn,Leu,Phe, 0.782; Gly,Ala,Arg,Val,Met,Leu, 0.779; Gln,Ala,Arg,Orn,Lys,Phe, 0.779; Gly,Ala,Arg,Pro,Met,Orn, 0.779; His,Ala,Cit,Met,Orn,Phe, 0.779; Gly,Ala,Arg,Pro,Met,Phe, 0.779; Thr,Ala,Tyr,Met,Orn,Phe, 0.777; Asn,Ala,Val,Orn,Phe,Trp, 0.775; Ala,Val,Met,Orn,Ile,Leu, 0.774; His,Ala,Arg,Ile,Leu,Phe, 0.772; Ala, Pro,Val, Orn, Leu, Phe, 0.772; Ala, Pro, Orn, Lys, Leu, Phe, 0.772; His,Ala,Val,Met,Orn,Leu, 0.77; Ala,Cit,Orn,Lys,Leu,Phe, 0.77; His,Ala,Orn,Ile,Phe,Trp, 0.77; Gly,Ala,Arg,Met,Lys,Phe, 0.77; Asn,Ala,Cit,Orn,Lys,Phe, 0.77; Ala,Cit,Val,Met,Leu,Phe, 0.77; Ala,Pro,Val,Orn,Lys,Phe, 0.77; Gly,Ala,Arg,Val,Met,Lys, 0.77; Gly,Thr,Ala,Arg,Met,Phe, 0.767; Gly,Ala,Arg,Val,Met,Phe, 0.765; Ala,Arg,Met,Lys,Leu,Trp, 0.764; Ser,Gly,Ala,Arg,Met,Phe, 0.762; Gln,Ala,Arg,Met,Ile,Trp, 0.762; Ser,Ala,Arg,Val,Met,Orn, 0.76; His,Ala,Arg,Tyr,Met,Ile, 0.758; Gly,Ala,Cit,Arg,Pro,Met, 0.758; Gln,Ala,Arg,Tyr,Met,Orn, 0.757; Ser,Thr,Ala,Arg,Met,Orn, 0.755; Asn,Gly,Ala,Arg,Met,Trp, 0.755; His,Ala,Arg,Pro,Met,Phe, 0.755; Gly,Ala,Arg,Met,Ile,Trp, 0.753; Ala,Arg,Tyr,Orn,Lys,Phe, 0.753; Gly,Ala,Cit,Arg,Val,Met, 0.753; Ser,Ala,Cit,Arg,Met,Leu, 0.752; Gln,Ala,Val,Met,Orn,Ile, 0.75; Asn,Ala,Arg,Tyr,Met,Ile, 0.75; Gly,His,Ala,Arg,Val,Met, 0.75; His,Ala,Met,Ile,Leu,Phe, 0.75; Thr,Ala,Cit,Arg,Met,Ile, 0.748; Gly,Gln,His,Ala,Arg,Phe, 0.747; Ser,Thr,Ala,Pro,Met,Orn, 0.747; Ala,Arg,Pro,Val,Orn,Phe, 0.745; Gln,Thr,Ala,Met,Orn,Leu, 0.745; Gly,Ala,Cit,Arg,Val,Phe, 0.745; Ala,Met,Orn,Lys,Leu,Trp, 0.745; Gly,Ala,Cit,Arg,Lys,Phe, 0.743; Gly,His,Ala,Arg,Phe,Trp, 0.742; His,Ala,Cit,Met,Orn,Leu, 0.74; Gln,Ala,Pro,Met,Orn,Leu, 0.738; Gln,His,Ala,Met,Orn,Leu, 0.736; Ser,Thr,Ala,Val,Met,Orn, 0.735; Thr,Ala,Met,Orn,Lys,Ile, 0.733; Ser,His,Ala,Cit,Leu,Phe, 0.733; Ala,Cit,Pro,Met,Orn,Leu, 0.731; Gln,Ala,Cit,Met,Orn,Trp, 0.731; His,Ala,Cit,Met,Orn,Ile, 0.73; His,Ala,Met,Orn,Ile,Leu, 0.73; His,Ala,Met,Orn,Lys,Ile, 0.726; Ser,Ala,Cit,Met,Orn,Trp, 0.72; Ser,His,Ala,Arg,Leu,Phe, 0.709

[316. Formulae with two explanatory variables obtained from amino acids and amino acid-related metabolites before the start of treatment with the response rate as evaluation index]

hTrp,NP, 0.742; AnthA,NP, 0.726; Kyn,NP, 0.725; Pro,NP, 0.725; Ala,hTrp, 0.725; Ala,AnthA, 0.723; QA,NP, 0.72; Ala,Kyn, 0.72; Thr,NP, 0.715; Orn,NP, 0.713; Arg,NP, 0.706; hKyn,NP, 0.704; Lys,NP, 0.703; Arg,hTrp, 0.703; hTrp,AnthA, 0.701; Cit,NP, 0.699; hKyn,AnthA, 0.699; AnthA,Kyn, 0.698; Orn,hTrp, 0.693; Orn,hKyn, 0.693; Ala,NP, 0.691; Trp,NP, 0.689; Arg,hKyn, 0.689; AnthA,QA, 0.686; Arg,QA, 0.684; Pro,hKyn, 0.682; Arg,AnthA, 0.682; Pro,Kyn, 0.681; hTrp,QA, 0.677; Pro,hTrp, 0.677; Arg,Kyn, 0.677; Ala,hKyn, 0.676; Ala,Orn, 0.676; Pro,AnthA, 0.674; Lys,Kyn, 0.672; hKyn,hTrp, 0.671; Thr,Arg, 0.671; Orn,QA, 0.666; Ala,Arg, 0.666; Cit,QA, 0.666; hTrp,Kyn, 0.664; Lys,AnthA, 0.662; Ala,QA, 0.662; Trp,hKyn, 0.66; Cit,hKyn, 0.654; Lys,hKyn, 0.652; Cit,hTrp, 0.652; Arg,Orn, 0.652; Cit,AnthA, 0.652; Trp,Kyn, 0.652; Ala,Lys, 0.652; Trp,AnthA, 0.652; Orn,Kyn, 0.65; Lys,hTrp, 0.65; Ala,Cit, 0.65; Trp,hTrp, 0.649; Orn,AnthA, 0.649; Thr,hTrp, 0.645; Arg,Trp, 0.642; Arg,Pro, 0.642; Thr,Ala, 0.642; Ala,Pro, 0.64; Arg,Lys, 0.639; Thr,hKyn, 0.637; Thr,AnthA, 0.637; Lys,QA, 0.635; hKyn,QA, 0.633; Pro,Orn, 0.633; Cit,Arg, 0.633; Ala,Trp, 0.633; Pro,QA, 0.632; Cit,Orn, 0.63; hKyn,Kyn, 0.63; Thr,Kyn, 0.628; Orn,Lys, 0.623; Cit,Kyn, 0.622; Orn,Trp, 0.618; Kyn,QA, 0.617; Trp,QA, 0.615; Thr,Orn, 0.611; Cit,Pro, 0.606; Thr,QA, 0.605; Thr,Lys, 0.595; Cit,Lys, 0.588; Thr,Cit, 0.586; Cit,Trp, 0.586; Pro,Trp, 0.584; Thr,Pro, 0.579; Lys,Trp, 0.571; Thr,Trp, 0.566

[317. Formulae with three explanatory variables obtained from amino acids and amino acid-related metabolites before the start of treatment with the response rate as evaluation index]

Ala,AnthA,Kyn, 0.772; hTrp,AnthA,NP, 0.764; Ala,hKyn,AnthA, 0.76; Pro,hTrp,NP, 0.755; Ala,hTrp,AnthA, 0.753; Ala,hTrp,Kyn, 0.753; hTrp,Kyn,NP, 0.75; Pro,hTrp,AnthA, 0.75; Ala,hTrp,NP, 0.75; Ala,AnthA,NP, 0.748; Arg,hTrp,NP, 0.747; hTrp,QA,NP, 0.745; Pro,AnthA,NP, 0.745; Pro,hTrp,Kyn, 0.743; Pro,hKyn,hTrp, 0.742; Ala,Orn,Kyn, 0.742; Ala,AnthA,QA, 0.74; Pro,hKyn,AnthA, 0.74; Thr,hTrp,NP, 0.738; Thr,Ala,AnthA, 0.738; hTrp,AnthA,Kyn, 0.736; Cit,hTrp,QA, 0.735; Pro,Kyn,NP, 0.735; Ala,Lys,AnthA, 0.735; Pro,AnthA,Kyn, 0.735; Orn,hKyn,hTrp, 0.733; hKyn,hTrp,NP, 0.733; Ala,Kyn,NP, 0.731; Lys,AnthA,NP, 0.73; hKyn,hTrp,AnthA, 0.73; Ala,Cit,hTrp, 0.73; hTrp,AnthA,QA, 0.728; Ala,hTrp,QA, 0.728; Ala,Arg,hTrp, 0.728; Trp,AnthA,NP, 0.728; Arg,AnthA,NP, 0.728; Ala,Cit,Kyn, 0.728; Pro,hTrp,QA, 0.726; Cit,AnthA,NP, 0.726; Ala,Trp,AnthA, 0.726; AnthA,Kyn,NP, 0.726; Pro,Orn,hKyn, 0.725; Arg,AnthA,Kyn, 0.725; AnthA,QA,NP, 0.725; Ala,Arg,Kyn, 0.725; Pro,QA,NP, 0.723; Ala,Arg,AnthA, 0.723; Ala,Orn,NP, 0.723; Ala,Trp,Kyn, 0.723; hKyn,AnthA,NP, 0.723; Ala,Pro,Kyn, 0.723; Cit,hTrp,AnthA, 0.721; Pro,hKyn,NP, 0.721; Pro,hKyn,Kyn, 0.721; Trp,hTrp,AnthA, 0.72; Ala,Pro,AnthA, 0.72; Orn,hTrp,AnthA, 0.718; Arg,Kyn,NP, 0.718; Ala,Cit,AnthA, 0.718; Ala,Orn,hKyn, 0.718; Ala,Orn,QA, 0.718; Ala,Lys,Kyn, 0.718; Trp,AnthA,Kyn, 0.716; Cit,hKyn,NP, 0.715; Trp,hTrp,NP, 0.713; Orn,hKyn,AnthA, 0.713; Pro,AnthA,QA, 0.713; Cit,Kyn,NP, 0.711; Orn,AnthA,NP, 0.711; Thr,Arg,AnthA, 0.709; Trp,hTrp,Kyn, 0.709; Arg,QA,NP, 0.709; Lys,AnthA,Kyn, 0.709; Lys,hTrp,AnthA, 0.706; Arg,AnthA,QA, 0.706; Ala,Pro,NP, 0.704; Cit,QA,NP, 0.704; Orn,AnthA,QA, 0.703; Trp,hKyn,hTrp, 0.701; Ala,Arg,NP, 0.701; Ala,Lys,NP, 0.701; Cit,hKyn,AnthA, 0.701; Ala,Trp,NP, 0.699; Arg,hKyn,NP, 0.699; Ala,QA,NP, 0.699; hKyn,AnthA,QA, 0.698; Thr,hKyn,AnthA, 0.698; Cit,Arg,AnthA, 0.696; Ala,hKyn,NP, 0.694; Trp,hKyn,NP, 0.693; AnthA,Kyn,QA, 0.693; Lys,AnthA,QA, 0.688; Thr,Pro,AnthA, 0.686; Arg,Pro,AnthA, 0.681; Arg,Trp,AnthA, 0.674; Thr,AnthA,QA, 0.674; Cit,Pro,AnthA, 0.672; Pro,Lys,AnthA, 0.671; Cit,AnthA,QA, 0.671; Pro,Trp,AnthA, 0.669

[318. Formulae with four explanatory variables obtained from amino acids and amino acid-related metabolites before the start of treatment with the response rate as evaluation index]

Ala,hTrp,AnthA,NP, 0.787; Ala,hTrp,AnthA,Kyn, 0.784; Thr,Ala,AnthA,Kyn, 0.784; Pro,hTrp,AnthA,NP, 0.782; Ala,Lys,hTrp,AnthA, 0.78; Ala,Pro,AnthA,Kyn, 0.78; Pro,hKyn,hTrp,AnthA, 0.779; Ala,Trp,AnthA,Kyn, 0.779; Ala,Arg,AnthA,Kyn, 0.775; Ala,Pro,hTrp,Kyn, 0.774; Pro,hKyn,hTrp,Kyn, 0.774; Ala,Lys,hTrp,NP, 0.774; Ala,Orn,hKyn,hTrp, 0.774; Thr,Ala,Pro,AnthA, 0.772; Ala,AnthA,Kyn,NP, 0.772; Ala,Orn,hTrp,AnthA, 0.772; Ala,Lys,AnthA,Kyn, 0.772; Thr,Ala,hTrp,AnthA, 0.77; Ala,hKyn,AnthA,Kyn, 0.77; Cit,hTrp,AnthA,NP, 0.77; Ala,Orn,AnthA,Kyn, 0.767; Ala,AnthA,Kyn,QA, 0.767; Ala,Lys,hTrp,Kyn, 0.767; Ala,Orn,hTrp,NP, 0.767; Ala,Orn,AnthA,NP, 0.765; Arg,hTrp,AnthA,NP, 0.765; Cit,hTrp,Kyn,NP, 0.765; Ala,Cit,hTrp,NP, 0.764; Ala,Orn,hTrp,QA, 0.762; Ala,hKyn,hTrp,NP, 0.762; Ala,hTrp,Kyn,NP, 0.76; Ala,Trp,hKyn,AnthA, 0.76; Pro,hKyn,AnthA,NP, 0.758; Arg,Pro,hTrp,Kyn, 0.758; hKyn,hTrp,AnthA,NP, 0.757; Thr,hTrp,AnthA,NP, 0.757; Thr,Ala,hTrp,NP, 0.757; Ala,Orn,Kyn,NP, 0.757; Pro,hTrp,QA,NP, 0.757; Ala,Cit,hKyn,AnthA, 0.755; Ala,Trp,AnthA,NP, 0.755; Pro,hTrp,AnthA,QA, 0.753; Ala,Pro,hKyn,AnthA, 0.753; Ala,Orn,hKyn,AnthA, 0.753; Ala,Lys,AnthA,QA, 0.753; Ala,hTrp,Kyn,QA, 0.753; Thr,Ala,Arg,AnthA, 0.753; Pro,hKyn,hTrp,NP, 0.753; Thr,Ala,Lys,AnthA, 0.753; Trp,hTrp,AnthA,Kyn, 0.753; Ala,AnthA,QA,NP, 0.752; Arg,Pro,hTrp,AnthA, 0.752; Pro,Trp,AnthA,NP, 0.752; Thr,Ala,Trp,AnthA, 0.752; Pro,Orn,hTrp,NP, 0.752; Cit,Pro,hTrp,NP, 0.752; Pro,hKyn,AnthA,Kyn, 0.75; Ala,Pro,hTrp,NP, 0.75; Arg,AnthA,Kyn,NP, 0.75; Arg,Pro,hKyn,AnthA, 0.747; Thr,Ala,hKyn,AnthA, 0.747; Pro,AnthA,Kyn,NP, 0.747; Ala,Orn,AnthA,QA, 0.747; Pro,AnthA,QA,NP, 0.747; Thr,Pro,hTrp,Kyn, 0.747; Ala,Orn,Trp,AnthA, 0.747; Pro,Trp,hTrp,Kyn, 0.747; Ala,hTrp,QA,NP, 0.747; Ala,Arg,Lys,AnthA, 0.747; Trp,hKyn,hTrp,AnthA, 0.745; Arg,Pro,AnthA,NP, 0.745; Lys,hKyn,hTrp,AnthA, 0.745; Pro,Lys,hTrp,Kyn, 0.745; Ala,Pro,Lys,AnthA, 0.745; Ala,Cit,Lys,AnthA, 0.745; Pro,Trp,hKyn,AnthA, 0.743; Ala,Arg,AnthA,NP, 0.743; Cit,Pro,hKyn,AnthA, 0.742; Ala,Pro,AnthA,NP, 0.742; Ala,Trp,AnthA,QA, 0.742; Cit,hTrp,AnthA,QA, 0.742; Ala,Lys,Trp,AnthA, 0.742; Pro,Lys,hKyn,AnthA, 0.738; Ala,Pro,AnthA,QA, 0.738; Cit,hKyn,AnthA,NP, 0.738; Ala,Arg,AnthA,QA, 0.736; Lys,hKyn,AnthA,NP, 0.736; Ala,Cit,Kyn,NP, 0.736; Pro,Trp,AnthA,Kyn, 0.735; Ala,Cit,Orn,AnthA, 0.735; hKyn,hTrp,AnthA,QA, 0.731; Trp,hKyn,AnthA,NP, 0.725; Thr,Trp,hKyn,AnthA, 0.721; Arg,Trp,hKyn,AnthA, 0.715; Trp,hKyn,AnthA,Kyn, 0.713; Cit,Trp,hKyn,AnthA, 0.711; Trp,hKyn,AnthA,QA, 0.711; Pro,Trp,AnthA,QA, 0.711; Lys,Trp,hKyn,AnthA, 0.706; Pro,Lys,AnthA,QA, 0.706

[319. Formulae with five explanatory variables obtained from amino acids and amino acid-related metabolites before the start of treatment with the response rate as evaluation index]

Thr,Ala,Cit,hTrp,Kyn, 0.801; Thr,Pro,hTrp,AnthA,NP, 0.797; Ala,Trp,hTrp,AnthA,NP, 0.797; Ala,Trp,hTrp,AnthA,Kyn, 0.796; Ala,Orn,Lys,hTrp,AnthA, 0.796; Ala,Cit,hTrp,Kyn,NP, 0.796; Ala,Lys,hTrp,AnthA,Kyn, 0.794; Thr,Pro,hKyn,hTrp,AnthA, 0.794; Ala,Lys,hTrp,AnthA,NP, 0.794; Ala,Pro,hTrp,AnthA,Kyn, 0.791; Thr,Ala,Orn,AnthA,Kyn, 0.791; Thr,Ala,Orn,hTrp,Kyn, 0.791; Thr,Ala,hTrp,AnthA,NP, 0.789; Pro,Trp,hKyn,hTrp,AnthA, 0.789; Ala,hTrp,AnthA,Kyn,QA, 0.789; Ala,hKyn,hTrp,AnthA,NP, 0.789; Ala, Pro,hTrp,AnthA,NP, 0.789; Ala,Arg,Orn,hTrp,Kyn, 0.789; Pro,Lys,hKyn,hTrp,AnthA, 0.787; Ala,Orn,hTrp,AnthA,NP, 0.787; Pro,Lys,hTrp,AnthA,NP, 0.787; Ala,Cit,Orn,hTrp,Kyn, 0.787; Pro,Orn,hKyn,hTrp,AnthA, 0.785; Arg,Pro,hTrp,AnthA,NP, 0.785; Cit,Pro,hTrp,AnthA,NP, 0.785; Ala,hTrp,AnthA,Kyn,NP, 0.784; Thr,Ala,Pro,hTrp,AnthA, 0.784; Ala,hTrp,AnthA,QA,NP, 0.784; Ala,Pro,Orn,hTrp,Kyn, 0.784; Thr,Ala,AnthA,Kyn,QA, 0.784; Ala,Pro,Trp,hTrp,Kyn, 0.784; Thr,Ala,Arg,AnthA,Kyn, 0.782; Ala,Cit,Arg,hTrp,Kyn, 0.782; Thr,Ala,AnthA,Kyn,NP, 0.78; Pro,Orn,hTrp,AnthA,NP, 0.78; Thr,Ala,Lys,hTrp,AnthA, 0.78; Ala,Pro,Lys,AnthA,Kyn, 0.78; Thr,Ala,Trp,AnthA,Kyn, 0.78; Ala,Cit,hKyn,hTrp,Kyn, 0.78; Thr,Pro,hTrp,AnthA,Kyn, 0.779; Pro,hTrp,AnthA,Kyn,QA, 0.779; Ala,Arg,Orn,hTrp,AnthA, 0.779; Ala,Pro,Orn,hTrp,AnthA, 0.779; Ala,Orn,Trp,AnthA,Kyn, 0.779; Ala,Pro,AnthA,Kyn,QA, 0.779; Pro,hKyn,hTrp,AnthA,QA, 0.777; Pro,hKyn,hTrp,AnthA,Kyn, 0.774; Arg,Pro,hKyn,hTrp,AnthA, 0.774; Pro,Trp,hTrp,AnthA,Kyn, 0.774; Thr,Pro,hTrp,AnthA,QA, 0.774; Ala,Trp,hTrp,AnthA,QA, 0.774; Ala,Cit,hTrp,AnthA,QA, 0.774; Ala,Cit,AnthA,Kyn,NP, 0.774; Ala,Lys,AnthA,Kyn,QA, 0.774; Ala,Arg,Lys,AnthA,Kyn, 0.774; Ala,Orn,AnthA,Kyn,QA, 0.772; Ala,Orn,hKyn,hTrp,AnthA, 0.772; Pro,Orn,hTrp,AnthA,Kyn, 0.772; Thr,Arg,Pro,hKyn,AnthA, 0.772; Pro,Lys,hTrp,AnthA,Kyn, 0.769; Cit,Pro,hTrp,AnthA,Kyn, 0.769; Thr,Pro,hKyn,AnthA,NP, 0.769; Thr,Ala,Arg,AnthA,NP, 0.769; Thr,Pro,Trp,hKyn,AnthA, 0.769; Ala,AnthA,Kyn,QA,NP, 0.769; Thr,Pro,hKyn,AnthA,QA, 0.769; Ala,Cit,Orn,hTrp,AnthA, 0.769; Thr,Cit,Pro,hKyn,AnthA, 0.769; Thr,Ala,hKyn,hTrp,AnthA, 0.767; Ala,Arg,hTrp,AnthA,QA, 0.767; Pro,Trp,hTrp,AnthA,QA, 0.767; Ala,Arg,AnthA,Kyn,NP, 0.767; Ala,Orn,hKyn,AnthA,Kyn, 0.767; Thr,Ala,Pro,AnthA,QA, 0.767; Ala,hTrp,Kyn,QA,NP, 0.767; Arg,Pro,Orn,hKyn,AnthA, 0.765; Cit,Pro,hTrp,AnthA,QA, 0.765; Ala,Cit,Orn,AnthA,Kyn, 0.765; Ala,Cit,hKyn,AnthA,Kyn, 0.765; Thr,Pro,hTrp,Kyn,NP, 0.764; Ala,Trp,hKyn,AnthA,NP, 0.764; Ala,Lys,AnthA,QA,NP, 0.764; Thr,Ala,Orn,AnthA,NP, 0.762; Thr,Ala,hTrp,Kyn,NP, 0.762; Ala,Cit,Pro,hTrp,AnthA, 0.762; Pro,Trp,hKyn,AnthA,NP, 0.76; Ala,Lys,hKyn,AnthA,NP, 0.758; Pro,hKyn,AnthA,QA,NP, 0.757; Thr,Ala,Trp,AnthA,NP, 0.757; Cit,Pro,Orn,hKyn,AnthA, 0.757; Pro,Orn,hKyn,AnthA,NP, 0.755; Ala,Pro,Orn,hKyn,AnthA, 0.755; Ala,Orn,Lys,AnthA,NP, 0.755; Ala,Arg,hKyn,AnthA,NP, 0.753; Ala,Arg,Orn,hKyn,AnthA, 0.753; Ala,Cit,Pro,hKyn,AnthA, 0.752; Ala,Arg,Pro,hTrp,AnthA, 0.748; Ala,Pro,hKyn,AnthA,QA, 0.748; Pro,Orn,hKyn,AnthA,QA, 0.748; Pro,Orn,Trp,hKyn,AnthA, 0.745

[320. Formulae with six explanatory variables obtained from amino acids and amino acid-related metabolites before the start of treatment with the response rate as evaluation index]

Thr,Ala,Orn,hTrp,AnthA,Kyn, 0.823; Ala,Arg,Orn,hTrp,AnthA,Kyn, 0.816; Ala,Pro,Trp,hTrp,AnthA,NP, 0.816; Thr,Ala,Cit,hTrp,AnthA,Kyn, 0.813; Thr,Pro,Orn,hKyn,hTrp,AnthA, 0.807; Thr,Ala,Orn,hTrp,AnthA,QA, 0.807; Thr,Ala,Pro,Orn,AnthA,Kyn, 0.807; Thr,Ala,Orn,Lys,hTrp,AnthA, 0.807; Ala,Cit,Trp,hTrp,Kyn,NP, 0.807; Ala,Orn,Trp,hTrp,AnthA,Kyn, 0.806; Ala,Arg,Orn,hTrp,AnthA,NP, 0.806; Ala,Orn,hTrp,AnthA,Kyn,QA, 0.806; Ala,Orn,Lys,hTrp,AnthA,QA, 0.806; Ala,Pro,Lys,hTrp,AnthA,Kyn, 0.802; Thr,Pro,hTrp,AnthA,Kyn,NP, 0.802; Thr,Ala,Pro,hKyn,hTrp,AnthA, 0.802; Thr,Ala,Cit,Orn,hTrp,Kyn, 0.802; Thr,Ala,Orn,hTrp,AnthA,NP, 0.801; Ala,Pro,Orn,hTrp,AnthA,Kyn, 0.801; Thr,Ala,Trp,hTrp,AnthA,Kyn, 0.801; Pro,Orn,Lys,hKyn,hTrp,AnthA, 0.801; Pro,Orn,Trp,hKyn,hTrp,AnthA, 0.801; Arg,Pro,Trp,hTrp,AnthA,NP, 0.801; Thr,Ala,hKyn,hTrp,AnthA,Kyn, 0.799; Thr,Cit,Pro,hKyn,hTrp,AnthA, 0.799; Ala,Cit,hTrp,AnthA,Kyn,NP, 0.797; Thr,Pro,Trp,hKyn,hTrp,AnthA, 0.797; Ala,Orn,Lys,hTrp,Kyn,NP, 0.797; Ala,Cit,hKyn,hTrp,AnthA,Kyn, 0.797; Ala,Trp,hTrp,AnthA,QA,NP, 0.797; Thr,Ala,Arg,Orn,hTrp,AnthA, 0.797; Thr,Ala,Pro,hTrp,AnthA,QA, 0.796; Ala,Lys,hKyn,hTrp,AnthA,NP, 0.796; Ala,Arg,Lys,hTrp,AnthA,NP, 0.796; Ala,Cit,hTrp,AnthA,Kyn,QA, 0.796; Ala,Arg,Trp,hTrp,AnthA,Kyn, 0.796; Ala,Trp,hTrp,AnthA,Kyn,QA, 0.796; Thr,Ala,Arg,Lys,hTrp,AnthA, 0.796; Thr,Ala,Cit,Orn,AnthA,Kyn, 0.796; Ala,Cit,Orn,hTrp,Kyn,NP, 0.796; Thr,Ala,hTrp,AnthA,Kyn,NP, 0.794; Thr,Ala,Cit,hTrp,Kyn,NP, 0.794; Thr,Ala,hTrp,AnthA,Kyn,QA, 0.794; Ala,Lys,hTrp,AnthA,Kyn,QA, 0.794; Thr,Ala,Cit,hTrp,AnthA,QA, 0.794; Thr,Ala,Cit,Lys,hTrp,Kyn, 0.794; Thr,Ala,Lys,hTrp,AnthA,Kyn, 0.792; Pro,Lys,hKyn,hTrp,AnthA,NP, 0.792; Thr,Pro,Lys,hTrp,AnthA,NP, 0.792; Thr,Arg,Pro,hTrp,AnthA,Kyn, 0.792; Ala,Cit,Arg,hTrp,Kyn,NP, 0.792; Ala,Cit,Lys,hTrp,AnthA,QA, 0.792; Thr,Ala,Cit,Lys,hTrp,AnthA, 0.792; Thr,Pro,hKyn,hTrp,AnthA,NP, 0.791; Cit,Pro,Lys,hKyn,hTrp,AnthA, 0.791; Thr,Ala,Pro,Lys,AnthA,Kyn, 0.791; Thr,Ala,Arg,Orn,AnthA,Kyn, 0.791; Ala,Cit,Arg,hTrp,AnthA,Kyn, 0.789; Ala,Pro,Lys,hTrp,AnthA,QA, 0.789; Ala,Orn,Lys,AnthA,Kyn,NP, 0.789; Ala,Pro,Lys,hKyn,hTrp,AnthA, 0.787; Thr,Cit,Pro,hTrp,AnthA,Kyn, 0.787; Thr,Ala,Orn,AnthA,QA,NP, 0.787; Ala,Cit,Orn,Lys,hTrp,Kyn, 0.787; Pro,Trp,hKyn,hTrp,AnthA,NP, 0.785; Ala,Arg,hKyn,hTrp,AnthA,Kyn, 0.785; Ala,Cit,Pro,Lys,hTrp,Kyn, 0.785; Ala,Pro,hTrp,AnthA,QA,NP, 0.785; Thr,Ala,Orn,Lys,AnthA,Kyn, 0.785; Thr,Ala,Lys,AnthA,Kyn,NP, 0.785; Arg,Pro,Orn,hTrp,AnthA,NP, 0.785; Cit,Pro,hTrp,AnthA,QA,NP, 0.785; Ala,Cit,Lys,hKyn,hTrp,Kyn, 0.785; Thr,Ala,Trp,hTrp,AnthA,NP, 0.784; Ala,Arg,hTrp,AnthA,Kyn,QA, 0.784; Thr,Ala,Pro,Lys,hTrp,Kyn, 0.784; Ala,Cit,hTrp,AnthA,QA,NP, 0.784; Thr,Ala,Trp,hTrp,AnthA,QA, 0.782; Thr,Ala,Cit,hKyn,AnthA,Kyn, 0.782; Thr,Ala,Orn,hKyn,AnthA,NP, 0.78; Thr,Ala,Pro,Orn,hKyn,AnthA, 0.779; Ala,Arg,Pro,Lys,hTrp,AnthA, 0.779; Ala,Arg,Pro,Lys,hTrp,Kyn, 0.779; Ala,Arg,Pro,hKyn,hTrp,AnthA, 0.777; Thr,Ala,Pro,hTrp,Kyn,NP, 0.777; Ala,Cit,hKyn,hTrp,AnthA,QA, 0.777; Ala,Pro,Orn,AnthA,Kyn,NP, 0.777; Ala,Arg,Orn,hKyn,hTrp,AnthA, 0.775; Arg,Pro,hTrp,AnthA,Kyn,QA, 0.775; Thr,Ala,Arg,hTrp,Kyn,NP, 0.775; Thr,Pro,Trp,hTrp,AnthA,Kyn, 0.774; Thr,Ala,Pro,Orn,AnthA,NP, 0.774; Ala,Cit,Arg,hTrp,AnthA,QA, 0.774; Arg,Pro,Orn,hKyn,AnthA,NP, 0.774; Ala,Pro,Orn,Lys,AnthA,Kyn, 0.774; Thr,Ala,Cit,hKyn,AnthA,NP, 0.772; Thr,Pro,Trp,hTrp,AnthA,QA, 0.77; Thr,Pro,hKyn,AnthA,QA,NP, 0.769; Thr,Ala,Trp,hKyn,hTrp,AnthA, 0.767; Thr,Ala,Pro,AnthA,QA,NP, 0.767

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

1. An evaluating method comprising:
an evaluating step of evaluating a pharmacological action of an immune checkpoint inhibitor in a subject to be evaluated, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hAnthA, hIAA, hKyn, hTrp, IAA, ILA, Kyn, KynA, NFKyn, NP, PA, QA, Serot, and XA in blood of the subject to be evaluated, or (ii) a value of a formula calculated using the concentration value and the formula including an explanatory variable to be substituted with the concentration value.

2. The evaluating method according to claim 1, wherein the blood is taken from the subject to be evaluated after or before treatment with the immune checkpoint inhibitor is started, and the evaluating step is to evaluate the effect of the treatment in the subject to be evaluated.

3. The evaluating method according to claim 1, wherein the blood is taken from the subject to be evaluated before treatment with the immune checkpoint inhibitor is started, and the evaluating step is to evaluate a risk of developing an adverse effect with the treatment in the subject to be evaluated.

4. The evaluating method according to claim 1, wherein the evaluating step is performed by a control unit of an information processing apparatus including the control unit.

5. A calculating method comprising:
a calculating step of calculating a value of a formula for evaluating a pharmacological action of an immune checkpoint inhibitor, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hAnthA, hIAA, hKyn, hTrp, IAA, ILA, Kyn, KynA, NFKyn, NP, PA, QA, Serot, and XA in blood of a subject to be evaluated, and (ii) the formula including an explanatory variable to be substituted with the concentration value.

6. The calculating method according to claim 5, wherein the blood is taken from the subject to be evaluated after or before treatment with the immune checkpoint inhibitor is started, and the formula is to evaluate an effect of the treatment.

7. The calculating method according to claim 5, wherein the blood is taken from the subject to be evaluated before treatment with the immune checkpoint inhibitor is started, and the formula is to evaluate a risk of developing an adverse effect with the treatment.

8. The calculating method according to claim 5, wherein the calculating step is performed by a control unit of an information processing apparatus including the control unit.

9. An evaluating apparatus comprising a control unit, wherein the control unit includes:
an evaluating unit that evaluates a pharmacological action of an immune checkpoint inhibitor in a subject to be evaluated, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hAnthA, hIAA, hKyn, hTrp, IAA, ILA, Kyn, KynA, NFKyn, NP, PA, QA, Serot, and XA in blood of the subject to be evaluated, or (ii) a value of a formula calculated using the concentration value and the formula including an explanatory variable to be substituted with the concentration value.

10. The evaluating apparatus according to claim 9, being communicatively connected via a network to a terminal apparatus that provides concentration data on the concentration value or the value of the formula,
wherein the control unit further includes:
a data receiving unit that receives the concentration data or the value of the formula transmitted from the terminal apparatus; and
a result transmitting unit that transmits an evaluation result obtained by the evaluating unit to the terminal apparatus,
wherein the evaluating unit uses the concentration value included in the concentration data or the value of the formula received by the data receiving unit.

11. A calculating apparatus comprising a control unit, wherein the control unit includes:
a calculating unit that calculates a value of a formula for evaluating a pharmacological action of an immune checkpoint inhibitor, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hAnthA, hIAA, hKyn, hTrp, IAA, ILA, Kyn, KynA, NFKyn, NP, PA, QA, Serot, and XA in blood of a subject to be evaluated, and (ii) the formula including an explanatory variable to be substituted with the concentration value.

12. An evaluating program for causing a control unit of an information processing apparatus including the control unit to perform:
an evaluating step of evaluating a pharmacological action of an immune checkpoint inhibitor in a subject to be evaluated, using (ii) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hAnthA, hIAA, hKyn, hTrp, IAA, ILA, Kyn, KynA, NFKyn, NP, PA, QA, Serot, and XA in blood of the subject to be evaluated, or (ii) a value of a formula calculated using the concentration value and the formula including an explanatory variable to be substituted with the concentration value.

13. A calculating program for causing a control unit of an information processing apparatus including the control unit to perform:
a calculating step of calculating a value of a formula for evaluating a pharmacological action of an immune checkpoint inhibitor, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hAnthA, hIAA, hKyn, hTrp, IAA, ILA, Kyn, KynA, NFKyn, NP, PA, QA, Serot, and XA in blood of a subject to be evaluated, and (ii) the formula including an explanatory variable to be substituted with the concentration value.

14. A computer readable recording medium storing the program according to claim 12.

15. An evaluating system comprising:
an evaluating apparatus including a control unit; and
a terminal apparatus including a control unit,
wherein the evaluating apparatus and the terminal apparatus are communicatively connected to each other via a network,
the control unit of the terminal apparatus includes:
a data transmitting unit that transmits, to the evaluating apparatus, (i) concentration data on a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hAnthA, hIAA, hKyn, hTrp, IAA, ILA, Kyn, KynA, NFKyn, NP, PA, QA, Serot, and XA in blood of a subject to be evaluated, or (ii) a value of a formula calculated using the concentration value and the formula including an explanatory variable to be substituted with the concentration value; and
a result receiving unit that receives an evaluation result transmitted from the evaluating apparatus on a pharmacological action of an immune checkpoint inhibitor, and
the control unit of the evaluating apparatus includes:
a data receiving unit that receives the concentration data or the value of the formula transmitted from the terminal apparatus;
an evaluating unit that evaluates a pharmacological action of an immune checkpoint inhibitor in the subject to be evaluated, using the concentration value included in the concentration data or the value of the formula received by the data receiving unit; and
a result transmitting unit that transmits the evaluation result obtained by the evaluating unit to the terminal apparatus.

16. A terminal apparatus comprising a control unit, wherein the control unit includes:
a result obtaining unit that obtains an evaluation result on a pharmacological action of an immune checkpoint inhibitor,
wherein the evaluation result is a result of evaluating a pharmacological action of an immune checkpoint inhibitor in a subject to be evaluated, using (i) a concentration value of at least one metabolite among Glu, Arg, Orn, Cit, His, Val, Phe, Tyr, Met, Pro, Asn, Leu, Lys, Thr, Ile, Gln, Ala, Ser, a-ABA, Trp, Gly, AnthA, hAnthA, hIAA, hKyn, hTrp, IAA, ILA, Kyn, KynA, NFKyn, NP, PA, QA, Serot, and XA in blood of the subject to be evaluated, or (ii) a value of a formula calculated using the concentration value and the formula including an explanatory variable to be substituted with the concentration value.

17. The terminal apparatus according to claim 16, being communicatively connected via a network to an evaluating apparatus that evaluates a pharmacological action of an immune checkpoint inhibitor,
wherein the control unit includes a data transmitting unit that transmits concentration data on the concentration value or the value of the formula to the evaluating apparatus, and the result obtaining unit receives the result transmitted from the evaluating apparatus.
